(19) United States
(12) Patent Application Publication
(10) Pub. No.: US 2007/0066587 A1
(43) Pub. Date: Mar. 22, 2007

(54) Substituted azetidine compounds, their preparation and use as medicaments

(75) Inventors: Rosa Cuberes Altisen, Barcelona (ES); Jordi Frigola Constansa, Barcelona (ES); Bonifacio Gutierrez Silva, Barcelona (ES)

Correspondence Address: OBLON, SPIVAK, MCCLELLAND, MAIER & NEUSTADT, P.C., 1940 DUKE STREET, ALEXANDRIA, VA, 22314, US

(73) Assignee: LABORATORIOS DEL DR. ESTEVE S.A, Barcelona (ES)

(21) Appl. No.: 11/505,463

(22) Filed: Aug. 17, 2006

Related U.S. Application Data
(63) Continuation-in-part of application No. PCT/EP05/01658, filed on Feb. 16, 2005.

(30) Foreign Application Priority Data
Feb. 17, 2004 (ES) P 2004 00379

(51) Int. Cl. A61K 31/397; A61K 31/433; A61K 31/4188; C07D 409/02; C07D 405/02; C07D 417/02

(52) U.S. Cl. 514/210.19; 514/210.2; 514/210.21; 548/950; 548/126; 548/303.1

(57) ABSTRACT

The present invention relates to substituted Azetidine compounds of general formula (I), methods for their preparation, medicaments comprising these compounds as well as their use for the preparation of a medicament for the treatment of humans and animals.

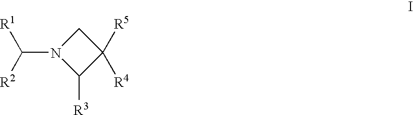

The present invention relates to substituted Azetidine compounds of general formula (I), methods for their preparation, medicaments comprising these compounds as well as their use for the preparation of a medicament for the treatment of humans and animals.

Cannabinoids are compounds, which are derived from the cannabis sativa plant which is commonly known as marijuana. The most active chemical compound of the naturally occurring cannabinoids is tetrahydrocannabinol (THC), particularly Δ⁹-THC.

These naturally occuring cannabinoids as well as their synthetic analogues promote their physiological effects via binding to specific G-coupled receptors, the so-called cannabinoid-receptors.

At present, two distinct types of receptors that bind both the naturally occurring and synthetic cannabinoids have been identified and cloned. These recepors, which are designated CB₁ and CB₂ are involved in a variety of physiological or pathophysiological processes in humans and animals, e.g. processes related to the central nervous system, immune system, cardiovascular system, endocrinous system, respiratory system, the gastrointestinal tract or to reproduction, as described for example, in Hollister, Pharm. Rev. 38, 1986, 1-20; Reny and Singha, Prog. Drug. Res., 36, 71-114, 1991; Consroe and Sandyk, in Marijuana/Cannabinoids, Neurobiology and Neurophysiology, 459, Murphy L. and Barthe A. Eds., CRC Press, 1992.

Therefore, compounds, which have a high binding affinity for these cannabinoid receptors and which are suitable for modulating these receptors are useful in the prevention and/or treatment of cannabinoid-receptor related disorders.

Thus, it was an object of the present invention to provide novel compounds that are in particular suitable as active substances in medicaments, preferably in medicaments for the modulation of Cannabinoid receptors, particularly Cannabinoid 1 (CB₁) receptors. Preferably said medicaments should be suitable for the prophylaxis and/or treatment of disorders related to the central nervous system, the immune system, the cardiovascular system, the endocrinous system, the respiratory system, the gastrointestinal tract or reproduction in humans and/or animals.

Said object was achieved by providing the substituted Azetidine compounds of general formula I given below, stereoisomers, corresponding N-oxides, corresponding salts and corresponding solvates thereof. Surprisingly, it has been found that these compounds have a high affinity for cannabinoid receptors, particularly for the CB₁-receptor and are therefore suitable for the prophylaxis and/or treatment of various disorders related to the central nervous system, the immune system, the cardiovascular system, the endocrinous system, the respiratory system, the gastrointestinal tract or reproduction in humans and/or-animals, preferably humans including infants, children and grown-ups.

Thus, in one of its aspects the present invention relates to substituted Azetidine compounds of general formula I, 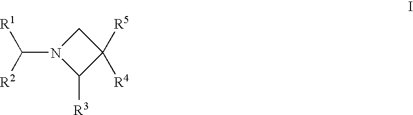
wherein

R¹ represents an optionally at least mono-substituted phenyl group,

R² represents a saturated or unsaturated, optionally at least mono-substituted, optionally at least one heteroatom as ring member containing cycloaliphatic group, which may be condensed with an optionally at least mono-substituted mono- or polycyclic ring system, or an optionally at least mono-substituted aryl or heteroaryl group, which may be condensed with an optionally at least mono-substituted mono- or polycyclic ring system

R³ represents a linear or branched, saturated or unsaturated, optionally at least mono-substituted aliphatic group, a saturated or unsaturated, optionally at least mono-substituted, optionally at least one heteroatom as ring member containing cycloaliphatic group, which may be condensed with a mono- or polycyclic ring-system and/or which may be bonded via a linear or branched alkylene group, or an optionally at least mono-substituted aryl or heteroaryl group, which may be condensed with a mono- or polycyclic ring system and/or which may be bonded via a linear or branched alkylene group, with the proviso that R³ is bonded to the azetidine ring via a carbon atom,

R⁴ represents a hydrogen atom, a cyano group, a carboxy group, a linear or branched alkyl group, or an optionally at least mono-substituted aryl group,

R⁵ represents an —O—SO₂—R⁶-moiety, an —NH—CO—R⁷-moiety, an —NH₂-moiety, an —NH—SO₂—R⁸ moiety, an —NR⁹—SO₂—R¹⁰-moiety or an —O—CO—R¹¹-moiety,

R⁶ represents a linear or branched, saturated or unsaturated, optionally at least mono-substituted aliphatic group, a saturated or unsaturated, optionally at least mono-substituted, optionally at least one heteroatom as ring member containing cycloaliphatic group, which may be condensed with a mono- or polycyclic ring-system and/or which may be bonded via a linear or branched alkylene group, or an optionally at least mono-substituted aryl or heteroaryl group, which may be condensed with a mono- or polycyclic ring system and/or which may be bonded via a linear or branched alkylene group,

R⁷ represents a linear or branched, saturated or unsaturated, optionally at least mono-substituted aliphatic group, a saturated or unsaturated, optionally at least mono-substituted, optionally at least one heteroatom as ring member containing cycloaliphatic group, which may be condensed with a mono- or polycyclic ring-system and/or which may be bonded via a linear or branched alkylene group, or an optionally at least mono-substituted aryl or heteroaryl group, which may be condensed with a mono- or polycyclic ring system and/or which may be bonded via a linear or branched alkylene group,

R⁸ represents a linear or branched, saturated or unsaturated, optionally at least mono-substituted aliphatic group, a saturated or unsaturated, optionally at least mono-substituted, optionally at least one heteroatom as ring member containing cycloaliphatic group, which may be condensed with a mono- or polycyclic ring-system and/or which may be bonded via a linear or bridged alkylene group, and/or which may be bridged by a linear or branched alkylene group, or an optionally at least mono-substituted aryl or heteroaryl group, which may be condensed with a mono- or polycyclic ring system and/or which may be bonded via a linear or branched alkylene group,

R⁹ represents an —SO₂—R¹²-moiety, a —CO—R¹³-moiety, a linear or branched, saturated or unsaturated, optionally at least mono-substituted aliphatic group, a saturated or unsaturated, optionally at least mono-substituted, optionally at least one heteroatom as ring member containing cycloaliphatic group, which may be condensed with a mono- or polycyclic ring-system and/or which may be bonded via a linear or branched alkylene group and/or bridged by a linear or branched alkylene group, or an optionally at least mono-substituted aryl or heteroaryl group, which may be condensed with a mono- or polycyclic ring system and/or which may be bonded via an alkylene group,

R¹⁰ represents a linear or branched, saturated or unsaturated, optionally at least mono-substituted aliphatic group, a saturated or unsaturated, optionally at least mono-substituted, optionally at least one heteroatom as ring member containing cycloaliphatic group, which may be condensed with a mono- or polycyclic ring-system and/or which may be bonded via a linear or bridged alkylene group, and/or which may be bridged by a linear or branched alkylene group, or an optionally at least mono-substituted aryl or heteroaryl group, which may be condensed with a mono- or polycyclic ring system and/or which may be bonded via a linear or branched alkylene group,

R¹¹ represents a linear or branched, saturated or unsaturated, optionally at least mono-substituted aliphatic group, a saturated or unsaturated, optionally at least mono-substituted, optionally at least one heteroatom as ring member containing cycloaliphatic group, which may be condensed with a mono- or polycyclic ring-system and/or which may be bonded via a linear or bridged alkylene group, and/or which may be bridged by a linear or branched alkylene group, or an optionally at least mono-substituted aryl or heteroaryl group, which may be condensed with a mono- or polycyclic ring system and/or which may be bonded via a linear or branched alkylene group,

R¹² represents a linear or branched, saturated or unsaturated, optionally at least mono-substituted aliphatic group, a saturated or unsaturated, optionally at least mono-substituted, optionally at least one heteroatom as ring member containing cycloaliphatic group, which may be condensed with a mono- or polycyclic ring-system and/or which may be bonded via a linear or bridged alkylene group, and/or which may be bridged by a linear or branched alkylene group, or an optionally at least mono-substituted aryl or heteroaryl group, which may be condensed with a mono- or polycyclic ring system and/or which may be bonded via a linear or branched alkylene group,

R¹³ represents a linear or branched, saturated or unsaturated, optionally at least mono-substituted aliphatic group, a saturated or unsaturated, optionally at least mono-substituted, optionally at least one heteroatom as ring member containing cycloaliphatic group, which may be condensed with a mono- or polycyclic ring-system and/or which may be bonded via a linear or bridged alkylene group, and/or which may be bridged by a linear or branched alkylene group, or an optionally at least mono-substituted aryl or heteroaryl group, which may be condensed with a mono- or polycyclic ring system and/or which may be bonded via a linear or branched alkylene group,

optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, a corresponding salt thereof, or a corresponding solvate thereof,

with the proviso that compounds of general formula I, in which R¹ and R² each represent an unsubstituted phenyl group, R⁵ represents an —O—SO₂—R⁶-moiety and R⁶ represents a methyl group are excluded.

A mono- or polycyclic ring-system according to the present invention means a mono- or polycyclic hydrocarbon ring-system that may be saturated, unsaturated or aromatic. If the ring system is polycyclic, each of its different rings may show a different degree of saturation, i.e. it may be saturated, unsaturated or aromatic. Optionally each of the rings of the mono- or polycyclic ring system may contain one or more, e.g. 1, 2 or 3, heteroatoms as ring members, which may be identical or different and which can preferably be selected from the group consisting of N, O, S and P, more preferably be selected from the group consisting of N, O and S. Preferably the polycyclic ring-system may comprise two rings that are condensed. The rings of the mono- or polycyclic ring-system are preferably 5- or 6-membered. The term “condensed” according to the present invention means that a ring or ring-system is attached to another ring or ring-system, whereby the terms “annulated” or “annelated” are also used by those skilled in the art to designate this kind of attachment.

If one or more of the residues R², R³ and R⁶—R¹³ represents or comprises a saturated or unsaturated, optionally at least one heteroatom as ring member containing cycloaliphatic group, which is substituted by one or more, e.g. 1, 2, 3, 4 or 5, substituents, unless defined otherwise, each of the substituents may be independently selected from the group consisting of hydroxy, fluorine, chlorine, bromine, branched or unbranched C₁₋₄-alkyl, branched or unbranched C₁₋₄-alkoxy, branched or unbranched C₁₋₄-perfluoroalkoxy, branched or unbranched C₁₋₄-perfluoroalkyl, oxo, amino, carboxy, amido, cyano, nitro, —SO₂NH₂, —CO—C₁₋₄-alkyl, —SO—C₁₋₄-alkyl, —SO₂—C₁₋₄-alkyl, —NH—SO₂—C₁₋₄-alkyl, wherein the C₁₋₄-alkyl may in each case be branched or unbranched, and a phenyl group, more preferably be selected from the group consisting of hydroxy, F, Cl, Br, methoxy, ethoxy, methyl, ethyl, oxo, CF₃ and a phenyl group.

If one or more of the residues R², R³ and R⁶—R¹³ represents or comprises a cycloaliphatic group, which contains one or more heteroatoms as ring members, unless defined otherwise, each of these heteroatoms may preferably be selected from the group consisting of N, O and S. Preferably a cycloaliphatic group may contain 1, 2 or 3 heteroatoms independently selected from the group consisting of N, O and S.

Suitable saturated or unsaturated, optionally at least heteroatom as ring member containing, optionally at least mono-substituted cycloaliphatic groups may preferably be selected from the group consisting of Cyclopropyl, Cyclobutyl, Cyclopentyl, Cyclohexyl, Cycloheptyl, Cyclooctyl, Cyclopentenyl, Cyclohexenyl, Cycloheptenyl, Cyclooctenyl, Pyrrolidinyl, Piperidinyl, Piperazinyl, Homo-Piperazinyl and Morpholinyl.

If one or more of the residues R², R³ and R⁶—R¹³ comprises a mono- or polycyclic ring system, which is substituted by one or more, e.g. 1, 2, 3, 4 or 5, substituents, unless defined otherwise, each of the substituents may be independently selected from the group consisting of hydroxy, fluorine, chlorine, bromine, branched or unbranched C₁₋₄-alkoxy, branched or unbranched C₁₋₄-alkyl, branched or unbranched C₁₋₄-perfluoroalkoxy, branched or unbranched. C₁₋₄-perfluoroalkyl, amino, carboxy, oxo, amido, cyano, nitro, —SO₂NH₂, —CO—C₁₋₄-alkyl, —SO—C₁₋₄-alkyl, —SO₂—C₁₋₄-alkyl, —NH—SO₂—C₁₋₄-alkyl , wherein the C₁₋₄-alkyl may in each case be branched or unbranched, and a phenyl group, more preferably be selected from the group consisting of hydroxy, F, Cl, Br, methyl, ethyl, methoxy, ethoxy, CF₃, oxo and a phenyl group.

If one or more of the residues R², R³, R⁴ and R⁶—R¹³ represents or comprises an aryl group, which is substituted by one or more, e.g. 1, 2, 3, 4 or 5, substituents, unless defined otherwise, each of the substituents may be independently selected from the group consisting of a halogen atom, a linear or branched C₁₋₆-alkyl group, a linear or branched C₁₋₆ alkoxy group, a formyl group, a hydroxy group, a trifluoromethyl group, a trifluoromethoxy group, a —CO—C₁₋₆alkyl group, a cyano group, a carboxy group, a —CO—O—C₁₋₆-alkyl group, a —CO—NR^{A}R^{B}-moiety, a CO—NH—NR^{C}R^{D}-moiety, an —S—C₁₋₆-alkyl group, an —SO—C₁₋₆-alkyl group, an —SO₂—C₁₋₆-alkyl group, a —C₁₋₆-alkylene-S—C₁₋₆-alkyl group, a —C₁₋₆-alkylene-SO—C₁₋₆-alkyl group, a —C₁₋₆-alkylene-SO₂—C₁₋₆-alkyl group, a C₁₋₆-alkyl group substituted by one or more hydroxy groups and a —C₁₋₆-alkylene-NR^{E}R^{F} group,

whereby R^{A}, R^{B}, identical or different, represent hydrogen or a C₁₋₆-alkyl group, or R^{A} and R^{B} together with the bridging nitrogen atom form a saturated, mono- or bicyclic, 3-10 membered heterocyclic ring system, which may be at least mono-substituted by one or more, identical or different, C₁₋₆ alkyl groups and/or which may contain at least one further heteroatom selected from the group consisting of nitrogen, oxygen and sulphur as a ring member,

R^{C}, R^{D}, identical or different, represent a hydrogen atom, a C₁₋₆-alkyl group, a —CO—O—C₁₋₆-alkyl group, a C₃₋₈-cycloalkyl group, a C₁₋₆-alkylene-C₃₋₈-cycloalkyl group, C₁₋₆-alkylene-O—C₁₋₆-alkyl group or a C₁₋₆-alkyl group substituted with one or more hydroxy groups, or R^{C}, R^{D} together with the bridging nitrogen atom form a saturated, mono- or bicyclic, 3-10 membered heterocyclic ring system, which may be at least mono-substituted by one or more substituents independently selected from the group consisting of C₁₋₆ alkyl group, a —CO—C₁₋₆-alkyl group, a —CO—O—C₁₋₆-alkyl group, a —CO—NH—C₁₋₆-alkyl group, a —CS—NH—C₁₋₆-alkyl group, an oxo group, a C₁₋₆-alkyl group substituted with one or more hydroxy groups, a C₁₋₆-alkylene-O—C₁₋₆-alkyl group and a —CO—NH₂ group and/or which may contain at least one further heteroatom selected from the group consisting of nitrogen, oxygen and sulphur as a ring member, and

wherein R^{E}, R^{F}, identical or different, represent hydrogen or a C₁₋₆-alkyl group, or R^{E} and R^{F} together with the bridging nitrogen atom form a saturated, mono- or bicyclic, 3-10 membered heterocyclic ring system, which may be at least mono-substituted by one or more, identical or different C₁₋₆ alkyl groups and/or which may contain at least one further heteroatom selected from the group consisting of nitrogen, oxygen and sulphur as a ring member.

Preferred aryl groups, which may optionally be at least mono-substituted, are phenyl and naphthyl.

If one or more of the residues R², R³ and R⁶—R¹³ represents or comprises a heteroaryl group, which is substituted by one or more, e.g. 1, 2, 3, 4 or 5, substituents, unless defined otherwise, each of the substituents may be independently selected from the group consisting of a halogen atom, a linear or branched C₁₋₆-alkyl group, a linear or branched C₁₋₆ alcoxy group, a formyl group, a hydroxy group, a trifluoromethyl group, a trifluoromethoxy group, a —CO—C₁₋₆-alkyl group, a cyano group, a carboxy group, a —CO—O—C₁₋₆-alkyl group, a —CO—NR^{A}R^{B}-moiety, a —CO—NH—NR^{C}R^{D}-moiety, an —S—C₁₋₆-alkyl group, an —SO—C₁₋₆-alkyl group, an —SO₂—C₁₋₆-alkyl group, a —C₁₋₆-alkylene-S—C₁₋₆-alkyl group, a —C₁₋₆-alkylene-SO—C₁₋₆-alkyl group, a —C₁₋₆-alkylene-SO₂—C₁₋₆-alkyl group, a C₁₋₆-alkyl group substituted by one or more hydroxy groups and a —C₁₋₆-alkylene-NR^{E}R^{F} group,

whereby R^{A}, R^{B}, identical or different, represent hydrogen or a C₁₋₆-alkyl group, or R^{A} and R^{B} together with the bridging nitrogen atom form a saturated, mono- or bicyclic, 3-10 membered heterocyclic ring system, which may be at least mono-substituted by one or more, identical or different, C₁₋₆ alkyl groups and/or which may contain at least one further heteroatom selected from the group consisting of nitrogen, oxygen and sulphur as a ring member,

R^{C}, R^{D}, identical or different, represent a hydrogen atom, a C₁₋₆-alkyl group, a —CO—O—C₁₋₆-alkyl group, a C₃₋₈-cycloalkyl group, a C₁₋₆-alkylene-C₃₋₈-cycloalkyl group, C₁₋₆-alkylene-O—C₁₋₆-alkyl group or a C₁₋₆-alkyl group substituted with one or more hydroxy groups, or R^{C}, R^{D} together with the bridging nitrogen atom form a saturated, mono- or bicyclic, 3-10 membered heterocyclic ring system, which may be at least mono-substituted by one or more substituents independently selected from the group consisting of C₁₋₆ alkyl group, a —CO—C₁₋₆-alkyl group, a —CO—O—C₁₋₆-alkyl group, a —CO—NH—C₁₋₆-alkyl group, a —CS—NH—C₁₋₆-alkyl group, an oxo group, a C₁₋₆-alkyl group substituted with one or more hydroxy groups, a C₁₋₆-alkylene-O—C₁₋₆-alkyl group and a —CO—NH₂ group and/or which may contain at least one further heteroatom selected from the group consisting of nitrogen, oxygen and sulphur as a ring member, and

wherein R^{E}, R^{F}, identical or different, represent hydrogen or a C₁₋₆-alkyl group, or R^{E} and R^{F} together with the bridging nitrogen atom form a saturated, mono- or bicyclic, 3-10 membered heterocyclic ring system, which may be at least mono-substituted by one or more, identical or different C₁₋₆ alkyl groups and/or which may contain at least one further heteroatom selected from the group consisting of nitrogen, oxygen and sulphur as a ring member,

The heteroatoms, which are present as ring members in the heteroaryl radical, may, unless defined otherwise, independently be selected from the group consisting of nitrogen, oxygen and sulphur. Preferably a heteroaryl radical may contain 1, 2 or 3 heteroatoms independently selected from the group consisting of nitrogen, oxygen and sulphur.

Suitable heteroaryl groups, which may optionally be at least mono-substituted, may preferably be selected from the group consisting of thienyl, furyl, pyrrolyl, pyridinyl, imidazolyl, pyrimidinyl, pyrazinyl, indolyl, chinolinyl, isochinolinyl, benzo[1,2,5]-thiodiazolyl, benzo[b]thiophenyl, benzo[b]furanyl, imidazo[2,1-b]thiazolyl and pyrazolyl, more preferably be selected from the group consisting of thienyl-, benzo[1,2,5]-thiodiazolyl, benzo[b]thiophenyl, imidazo[2,1-b]thiazolyl and pyrazolyl.

If one or more of the residues R³ and R⁶—R¹³ represents or comprises a linear or branched, saturated or unsaturated aliphatic group, such as an alkyl group, which is substituted by one or more, e.g. 1, 2, 3, 4 or 5 substituents, unless defined otherwise, each of the substituents may be independently selected from the group consisting of hydroxy, fluorine, chlorine, bromine, branched or unbranched C₁₋₄-alkoxy, branched or unbranched C₁₋₄-perfluoroalkoxy, branched or unbranched C₁₋₄-perfluoroalkyl, amino, carboxy, amido, cyano, nitro, —SO₂NH₂, —CO—C₁₋₄-alkyl, —SO—C₁₋₄alkyl, —SO₂—C₁₋₄-alkyl, —NH—SO₂—C₁₋₄-alkyl , wherein the C₁₋₄-alkyl may in each case be branched or unbranched, and a phenyl group, more preferably be selected from the group consisting of hydroxy, F, Cl, Br, methoxy, ethoxy, CF₃ and a phenyl group.

Preferred linear or branched, saturated or unsaturated aliphatic groups, which may be substituted by one or more substituents, may preferably be selected from the group consisting of methyl, ethyl, n-propyl, isopropyl, n-butyl, iso-butyl, sec-butyl, tert-butyl, n-pentyl, n-hexyl, n-heptyl, n-octyl, n-nonyl, n-decyl, vinyl, ethinyl, propenyl, propinyl, butenyl and butinyl.

If any of the residues R³ and R⁶—R¹³ comprises a linear or branched alkylene group, unless defined otherwise, said alkylene group may preferably be selected from the group consisting of methylene (—(CH₂)—), ethylene (—(CH₂)₂—), n-propylene (—(CH₂)₃—), isopropylene (—(C(CH₃)₂—), n-butylene (—(CH₂)₄—), n-pentylene (—(CH₂)₅—), n-hexylene (—(CH₂)₆—), n-heptylene (—CH₂)—), n-octylene (—CH₂)₈—), n-nonylene(—(CH₂)₉—) and n-decylene (—(CH₂)₁₀—), more preferably from the group consisting of (—(CH₂)—), ethylene (—(CH₂)₂—), n-propylene (—CH₂)₃—), isopropylene (—(C(CH₃)₂—) and n-butylene (—(CH₂)₄—).

Preferred are compounds of general formula I given above, wherein R¹ represents a phenyl group, which is optionally substituted by one or more substituents independently selected from the group consisting of a halogen atom, a linear or branched C₁₋₆-alkyl group, a linear or branched C₁₋₆ alkoxy group, a formyl group, a hydroxy group, a trifluoromethyl group, a trifluoromethoxy group, a —CO—C₁₋₆-alkyl group, a cyano group, a carboxy group, a —CO—O—C₁₋₆-alkyl group, a —CO—NR^{A}R^{B}-moiety, a —CO—NH—NR^{C}R^{D}-moiety, an —S—C₁₋₆-alkyl group, an —SO—C₁₋₆-alkyl group, an —SO₂—C₁₋₆-alkyl group, a —C₁₋₆-alkylene-S—C₁₋₆-alkyl group, a —C₁₋₆-alkylene-SO—C₁₋₆-alkyl group, a —C₁₋₆-alkylene-SO₂—C₁₋₆-alkyl group, a C₁₋₆-alkyl group substituted by one or more hydroxy groups and a —C₁₋₆-alkylene-NR^{E}R^{F} group,

whereby R^{A}, R^{B}, identical or different, represent hydrogen or a C₁₋₆alkyl group, or R^{A} and R^{B} together with the bridging nitrogen atom form a saturated, mono- or bicyclic, 3-10 membered heterocyclic ring system, which may be at least mono-substituted by one or more, identical or different, C₁₋₆ alkyl groups and/or which may contain at least one further heteroatom selected from the group consisting of nitrogen, oxygen and sulphur as a ring member,

R^{C}, R^{D}, identical or different, represent a hydrogen atom, a C₁₋₆-alkyl group, a —CO—O—C₁₋₆-alkyl group, a C₃₋₈-cycloalkyl group, a C₁₋₆-alkylene-C₃₋₈-cycloalkyl group, C₁₋₆-alkylene-O—C₁₋₆-alkyl group or a C₁₋₆-alkyl group substituted with one or more hydroxy groups, or R^{C}, R^{D} together with the bridging nitrogen atom form a saturated, mono- or bicyclic, 3-10 membered heterocyclic ring system, which may be at least mono-substituted by one or more substituents independently selected from the group consisting of C₁₋₆ alkyl group, a —CO—C₁₋₆-alkyl group, a —CO—O—C₁₋₆-alkyl group, a —CO—NH—C₁₋₆-alkyl group, a —CS—NH—C₁₋₆-alkyl group, an oxo group, a C₁₋₆-alkyl group substituted with one or more hydroxy groups, a C₁₋₆alkylene-O—C₁₋₆-alkyl group and a —CO—NH₂ group and/or which may contain at least one further heteroatom selected from the group consisting of nitrogen, oxygen and sulphur as a ring member, and

wherein R^{E}, R^{F}, identical or different, represent hydrogen or a C₁₋₆-alkyl group, or R^{E} and R^{F} together with the bridging nitrogen atom form a saturated, mono- or bicyclic, 3-10 membered heterocyclic ring system, which may be at least mono-substituted by one or more, identical or different C₁₋₆ alkyl groups and/or which may contain at least one further heteroatom selected from the group consisting of nitrogen, oxygen and sulphur as a ring member,

preferably R¹ represents a phenyl group, which is optionally substituted by one or more substituents independently selected from the group consisting of a fluorine atom, a chlorine atom, a bromine atom, a linear or branched C₁₋₆-alkyl group, a linear or branched C₁₋₆ alkoxy group, a formyl group, a hydroxy group, a trifluoromethyl group, a trifluoromethoxy group, a cyano group and a carboxy group,

more preferably R¹ represents a phenyl group, which is optionally substituted by one or more substituents independently selected from the group consisting of a fluorine atom, a chlorine atom, a bromine atom, a methyl group, a methoxy group, a trifluoromethyl group and a trifluoromethoxy group, most preferably R¹ represents a phenyl group, which is substituted by a chlorine atom in the 4-position,

and the residues R²—R¹³ have the meaning given above, optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, a corresponding salt thereof, or a corresponding solvate thereof.

Also preferred are compounds of general formula I given above, wherein R² represents a saturated or unsaturated, optionally at least mono-substituted, optionally at least heteroatom as ring member containing C₃₋₈ cycloaliphatic group, which may be condensed with an optionally at least mono-substituted mono- or polycyclic ring system, or an optionally at least mono-substituted, 5- or 6-membered aryl or heteroaryl group, which may be condensed with an optionally at least mono-substituted mono- or polycyclic ring system,

preferably R² represents a phenyl group, which is optionally substituted by one or more substituents independently selected from the group consisting of a halogen atom, a linear or branched C₁₋₆-alkyl group, a linear or branched C₁₋₆ alcoxy group, a formyl group, a hydroxy group, a trifluoromethyl group, a trifluoromethoxy group, a —CO—C₁₋₆-alkyl group, a cyano group, a carboxy group, a —CO—O—C₁₋₆-alkyl group, a —CO—NR^{A}R^{B}-moiety, a —CO—NH—NR^{C}R^{D}-Moiety, an —S—C₁₋₆-alkyl group, an —SO—C₁₋₆-alkyl group, an —SO₂—C₁₋₆-alkyl group, a —C₁₋₆-alkylene-S—C₁₋₆-alkyl group, a —C₁₋₆-alkylene-SO—C₁₋₆-alkyl group, a —C₁₋₆-alkylene-SO₂—C₁₋₆-alkyl group, a C₁₋₆-alkyl group substituted by one or more hydroxy groups and a —C₁₋₆-alkylene-NR^{E}R^{F} group,

whereby R^{A}, R^{B}, identical or different, represent hydrogen or a C₁₋₆-alkyl group, or R^{A} and R^{B} together with the bridging nitrogen atom form a saturated, mono- or bicyclic, 3-10 membered heterocyclic ring system, which may be at least mono-substituted by one or more, identical or different, C₁₋₆ alkyl groups and/or which may contain at least one further heteroatom selected from the group consisting of nitrogen, oxygen and sulphur as a ring member,

R^{C}, R^{D}, identical or different, represent a hydrogen atom, a C₁₋₆-alkyl group, a —CO—O—C₁₋₆-alkyl group, a C₃₋₈-cycloalkyl group, a C₁₋₆-alkylene-C₃₋₈-cycloalkyl group, C₁₋₆alkylene-O—C₁₋₆-alkyl group or a C₁₋₆-alkyl group substituted with one or more hydroxy groups, or R^{C}, R^{D} together with the bridging nitrogen atom form a saturated, mono- or bicyclic, 3-10 membered heterocyclic ring system, which may be at least mono-substituted by one or more substituents independently selected from the group consisting of C₁₋₆ alkyl group, a —CO—C₁₋₆-alkyl group, a —CO—O—C₁₋₆-alkyl group, a —CO—NH—C₁₋₆-alkyl group, a —CS—NH—C₁₋₆-alkyl group, an oxo group, a C₁₋₆-alkyl group substituted with one or more hydroxy groups, a C₁₋₆-alkylene-O—C₁₋₆-alkyl group and a —CO—NH₂ group and/or which may contain at least one further heteroatom selected from the group consisting of nitrogen, oxygen and sulphur as a ring member, and

wherein R^{E}, R^{F}, identical or different, represent hydrogen or a C₁₋₆-alkyl group, or R^{E} and R^{F} together with the bridging nitrogen atom form a saturated, mono- or bicyclic, 3-10 membered heterocyclic ring system, which may be at least mono-substituted by one or more, identical or different C₁₋₆ alkyl groups and/or which may contain at least one further heteroatom selected from the group consisting of nitrogen, oxygen and sulphur as a ring member,

more preferably R² represents a phenyl group, which is optionally substituted by one or more substituents independently selected from the group consisting of a fluorine atom, a chlorine atom, a bromine atom, a methyl group, a methoxy group, a trifluoromethyl group and a trifluoromethoxy group,

most preferably R² represents a phenyl group, which is substituted by a chlorine atom in the 4-position,

and R¹ and R³—R¹³ have the meaning given above, optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, a corresponding salt thereof, or a corresponding solvate thereof.

Preference is also given to compounds of formula I given above, wherein R³ represents a linear or branched, saturated or unsaturated, optionally at least mono-substituted C₁₋₁₀-aliphatic group, a saturated or unsaturated, optionally at least mono-substituted, optionally at least one heteroatom as ring member containing C₃₋₈-cycloaliphatic group, which may be condensed with a mono- or polycyclic ring-system and/or which may be bonded via a linear or branched C₁₋₆-alkylene group, or an optionally at least mono-substituted, 5- or 6-membered aryl or heteroaryl group, which may be condensed with a mono- or polycyclic ring system and/or which may be bonded via a linear or branched C₁₋₆-alkylene group,

preferably R³ represents a linear or branched, optionally at least mono-substituted C₁₋₁₀-alkyl group, or an optionally at least mono-substituted, 5- or 6-membered aryl or heteroaryl group, which may be condensed with a mono- or polycyclic ring system and/or which may be bonded via a linear or branched C₁₋₆-alkylene group, more preferably R³ represents a linear or branched, unsubstituted C₁₋₁₀-alkyl group, most preferably R³ represents a methyl group,

and R¹, R² and R⁴—R¹³ have the meaning given above, optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, a corresponding salt thereof, or a corresponding solvate thereof.

Further preferred compounds of general formula I given above are compounds, in which R⁴ represents a hydrogen atom, a cyano group, a carboxy group, a linear or branched C₁₋₁₀-alkyl group, or an optionally at least mono-substituted, 5- or 6-membered aryl group, preferably R⁴ represents a hydrogen atom, a linear or branched C₁₋₃-alkyl group, or an optionally at least mono-substituted phenyl group, more preferably R⁴ represents a hydrogen atom or a linear or branched C₁₋₃-alkyl group, most preferably R⁴ represents a hydrogen atom, and R¹—R³ and R⁵—R¹³ have the meaning given above, optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, a corresponding salt thereof, or a corresponding solvate thereof.

Also preferred are compounds of general formula I given above, in which R⁵ represents an —O—SO₂—R⁶-moiety, an —NH—CO—R⁷-moiety, an —NH₂-moiety, an —NH—SO₂—R⁸ moiety or an —NR⁹—SO₂—R¹⁰-moiety, preferably R⁵ represents an —O—SO₂—R⁶-moiety, an —NH—SO₂—R⁸ moiety or an —NR⁹—SO₂—R¹⁰-moiety, and R¹—R⁴ and R⁶—R¹³ have the meaning given above, optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, a corresponding salt thereof, or a corresponding solvate thereof.

Preference is also given to compounds of general formula I given above, wherein R⁶ represents a linear or branched, saturated or unsaturated, optionally at least mono-substituted C₁₋₁₀ aliphatic group, a saturated or unsaturated, optionally at least mono-substituted, optionally at least one heteroatom as ring member containing C₃₋₈-cycloaliphatic group, which may be condensed with a mono- or polycyclic ring-system and/or which may be bonded via a linear or branched C₁₋₆-alkylene group, or an optionally at least mono-substituted, 5- or 6-membered aryl or heteroaryl group, which may be condensed with a mono- or polycyclic ring system and/or which may be bonded via a linear or branched C₁₋₆-alkylene group, preferably R⁶ represents an optionally at least mono-substituted C₃₋₈-cycloaliphatic group or an optionally at least mono-substituted phenyl group, wherein the respective substituents are independently selected from the group consisting of a fluorine atom, a chlorine atom, a bromine atom, a linear or branched C₁₋₆-alkyl group, a linear or branched C₁₋₆ alcoxy group, a formyl group, a hydroxy group, a trifluoromethyl group, a trifluoromethoxy group, a cyano group and a carboxy group, more preferably R⁶ represents a phenyl group, which is optionally substituted by one or more substituents independently selected from the group consisting of a fluorine atom, a chlorine atom, a bromine atom, a methyl group, a methoxy group, a trifluoromethyl group and a trifluoromethoxy group, and R¹—R⁵ and R⁷—R¹³ have the meaning given above, optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, a corresponding salt thereof, or a corresponding solvate thereof.

Furthermore, compounds of general formula I given above are preferred, in which R⁷ represents a linear or branched, saturated or unsaturated, optionally at least mono-substituted C₁₋₁₀-aliphatic group, a saturated or unsaturated, optionally at least mono-substituted, optionally at least one heteroatom as ring member containing C₃₋₈-cycloaliphatic group, which may be condensed with a mono- or polycyclic ring-system and/or which may be bonded via a linear or branched C₁₋₆-alkylene group, or an optionally at least mono-substituted aryl or heteroaryl group, which may be condensed with a mono- or polycyclic ring system and/or which may be bonded via a linear or branched C₁₋₆-alkylene group, preferably-R⁷ represents a linear or branched, optionally at least mono-substituted C₁₋₅-alkyl group, a saturated, optionally at least mono-substituted C₅₋₆-cycloaliphatic group, or an optionally at least mono-substituted phenyl group, more preferably R⁷ represents a linear or branched, optionally at least mono-substituted C₁₋₅-alkyl group, a saturated, optionally at least mono-substituted C₅₋₆-cycloaliphatic group, or an optionally at least mono-substituted phenyl group, wherein in each case the substituents are independently from one another selected from the group consisting of group consisting of a fluorine atom, a chlorine atom, a bromine atom, a methyl group, a methoxy group, a trifluoromethyl group and a trifluoromethoxy group, and R¹—R⁶ and R⁸—R¹³ have the meaning given above, optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, a corresponding salt thereof, or a corresponding solvate thereof.

Also preferred are compound of general formula I given above, wherein R⁸ represents a linear or branched, saturated or unsaturated, optionally at least mono-substituted C₁₋₁₀-aliphatic group, a saturated or unsaturated, optionally at least mono-substituted, optionally at least one heteroatom as ring member containing C₃₋₈-cycloaliphatic group, which may be condensed with a mono- or polycyclic ring-system and/or which may be bonded via a linear or bridged C₁₋₁₀-alkylene group and/or which may be bridged by a linear or branched C₁₋₅-alkylene group, or an optionally at least mono-substituted 5- or 6-membered aryl or heteroaryl group, which may be condensed with a mono- or polycyclic ring system and/or which may be bonded via a linear or branched C₁₋₁₀-alkylene group, preferably R⁸ represents a linear or branched C₁₋₁₀-alkyl group, a saturated or unsaturated, optionally at least mono-substituted, optionally at least one heteroatom as ring member containing C₅₋₆-cycloaliphatic group, which may be condensed with a mono- or polycyclic ring-system and/or which may be bonded via a linear or bridged C₁₋₃-alkylene group and/or which may be bridged by a linear or branched C₁₋₃-alkylene group, or an optionally at least mono-substituted 5- or 6-membered aryl or heteroaryl group, which may be condensed with a mono- or polycyclic ring system and/or which may be bonded via a linear or branched C₁₋₃-alkylene group, more preferably R⁸ represents a methyl group, an ethyl group, an n-propyl group, an n-butyl group, an optionally at least mono-substituted phenyl group, an optionally at least mono-substituted benzyl group, an optionally at least mono-substituted naphthyl group, which may be bonded via a C₁₋₃-alkylene group, an optionally at least mono-substituted thienyl group, an optionally at least mono-substituted 2,1,3-Benzothiadiazole group, an optionally at least mono-substituted Benzo[b]thiophenyl group, an optionally at least mono-substituted Imidazo[2.1-b]thiazole group, an optionally at least mono-substituted 1H-pyrazole group or a 7,7-Dimethyl-2-oxo-bicyclo-[2.2.1]-hept-1-yl group, most preferably R⁸ represents a methyl group, an ethyl group, an n-propyl group, an n-butyl group, an optionally at least mono-substituted phenyl group, an optionally at least mono-substituted benzyl group, an optionally at least mono-substituted naphthyl group, which may be bonded via a C₁₋₃-alkylene group, an optionally at least mono-substituted thienyl group, an optionally at least mono-substituted 2,1,3-Benzothiadiazole group, an optionally at least mono-substituted Benzo[b]thiophenyl group, an optionally at least mono-substituted Imidazo[2.1-b]thiazole group, an optionally at least mono-substituted 1H-pyrazole group or a 7,7-Dimethyl-2-oxo-bicyclo-[2.2.1]-hept-1-yl group, wherein said substituents, if present, are identical or different and selected from the group consisting of a fluorine atom, a chlorine atom, a bromine atom, a methyl group, a formyl group, a phenyl group, a phenoxy group, a phenoxy group substituted with bromine in the 4-position and a methylsulfonyl group, and R¹—R⁷ and R⁹—R¹³ have the meaning given above, optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, a corresponding salt thereof, or a corresponding solvate thereof.

Also preferred are compounds of general formula I given above, in which R⁹ represents an —SO₂—R¹²-moiety, a —CO—R¹³-moiety, a linear or branched, saturated or unsaturated, optionally at least mono-substituted C₁₋₁₀ aliphatic group, a saturated or unsaturated, optionally at least mono-substituted, optionally at least heteroatom as ring member containing C₃₋₈ cycloaliphatic group, which may be condensed with a mono- or polycyclic ring-system and/or which may be bonded by a linear or branched C₁₋₆ alkylene group and/or bridged by a linear or branched C₁₋₆ alkylene group, or an optionally at least mono-substituted aryl or heteroaryl group, which may be condensed with a mono- or polycyclic ring system and/or which may be bonded via a C₁₋₆ alkylene group, preferably R⁹ represents an —SO₂—R¹²-moiety, a linear or branched C₁₋₁₀ alkyl group, or an optionally at least mono-substituted phenyl group, which may be bonded via a C₁₋₂ alkylene group, more preferably R⁹ represents an —SO₂—R¹²-moiety, a linear or branched C₁₋₃ alkyl group, or a phenyl group, which may be bonded via a C₁₋₂ alkylene group and/or substituted with one or more substituents independently selected from the group consisting of a fluorine atom, a chlorine atom and a bromine atom, and R¹—R⁸ and R¹⁰—R¹³ have the meaning given above optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, a corresponding salt thereof, or a corresponding solvate thereof.

Preference is furthermore given to compounds of general formula I given above, wherein R¹⁰ represents a linear or branched, saturated or unsaturated, optionally at least mono-substituted C¹⁻¹⁰-aliphatic group, a saturated or unsaturated, optionally at least mono-substituted, optionally at least one heteroatom as ring member containing C₃₋₈-cycloaliphatic group, which may be condensed with a mono- or polycyclic ring-system and/or which may be bonded via a linear or bridged C₁₋₁₀-alkylene group and/or which may be bridged by a linear or branched C₁₋₅-alkylene group, or an optionally at least mono-substituted 5- or 6-membered aryl or heteroaryl group, which may be condensed with a mono- or polycyclic ring system and/or which may be bonded via a linear or branched C₁₋₁₀-alkylene group, preferably R¹⁰ represents a linear or branched C₁₋₁₀-alkyl group, a saturated or unsaturated, optionally at least mono-substituted, optionally at least one heteroatom as ring member containing C₅₋₆-cycloaliphatic group, which may be condensed with a mono- or polycyclic ring-system and/or which may be bonded via a linear or bridged C₁₋₃-alkylene group and/or which may be bridged by a linear or branched C₁₋₃-alkylene group, or an optionally at least mono-substituted 5- or 6-membered aryl or heteroaryl group, which may be condensed with a mono- or polycyclic ring system and/or which may be bonded via a linear or branched C₁₋₃-alkylene group, more preferably R¹⁰ represents a methyl group, an ethyl group, an n-propyl group, an n-butyl group, an optionally at least mono-substituted phenyl group, an optionally at least mono-substituted benzyl group, an optionally at least mono-substituted naphthyl group, which may be bonded via a C₁₋₃-alkylene group, an optionally at least mono-substituted thienyl group, an optionally at least mono-substituted 2,1,3-Benzothiadiazole group, an optionally at least mono-substituted Benzo[b]thiophenyl group, an optionally at least mono-substituted Imidazo[2.1-b]thiazole group, an optionally at least mono-substituted 1H-pyrazole group or a 7,7-Dimethyl-2-oxo-bicyclo-[2.2.1]-hept-1-yl group, most preferably R¹⁰ represents a methyl group, an ethyl group, an n-propyl group, an n-butyl group, an optionally at least mono-substituted phenyl group, an optionally at least mono-substituted benzyl group, an optionally at least mono-substituted naphthyl group, which may be bonded via a C₁₋₃-alkylene group, an optionally at least mono-substituted thienyl group, an optionally at least mono-substituted 2,1,3-Benzothiadiazole group, an optionally at least mono-substituted Benzo[b]thiophenyl group, an optionally at least mono-substituted Imidazo[2.1-b]thiazole group, an optionally at least mono-substituted 1H-pyrazole group or a 7,7-Dimethyl-2-oxo-bicyclo-[2.2.1]-hept-1-yl group, wherein said substituents, if present, are identical or different and selected from the group consisting of a fluorine atom, a chlorine atom, a bromine atom, a methyl group, a formyl group, a phenyl group, a phenoxy group, a phenoxy group substituted with bromine in the 4-position and a methylsulfonyl group, and R¹—R⁹ and R¹¹—R¹³ have the meaning given above, optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, a corresponding salt thereof, or a corresponding solvate thereof.

Furthermore, compounds of general formula I given above are preferred, wherein R¹¹ represents a linear or branched, saturated or unsaturated, optionally at least mono-substituted C₁₋₁₀-aliphatic group, a saturated or unsaturated, optionally at least mono-substituted, optionally at least one heteroatom as ring member containing C₃₋₈-cycloaliphatic group, which may be condensed with a mono- or polycyclic ring-system and/or which may be bonded via a linear or bridged C₁₋₁₀-alkylene group and/or which may be bridged by a linear or branched C₁₋₅alkylene group, or an optionally at least mono-substituted 5- or 6-membered aryl or heteroaryl group, which may be condensed with a mono- or polycyclic ring system and/or which may be bonded via a linear or branched C₁₋₁₀-alkylene group, preferably R¹¹ represents a linear or branched C₁₋₁₀-alkyl group, a saturated or unsaturated, optionally at least mono-substituted, optionally at least one heteroatom as ring member containing C₅₋₆-cycloaliphatic group, which may be condensed with a mono- or polycyclic ring-system and/or which may be bonded via a linear or bridged C₁₋₃-alkylene group and/or which may be bridged by a linear or branched C₁₋₃-alkylene group, or an optionally at least mono-substituted 5- or 6-membered aryl or heteroaryl group, which may be condensed with a mono- or polycyclic ring system and/or which may be bonded via a linear or branched C₁₋₃-alkylene group, more preferably R¹¹ represents a methyl group, an ethyl group, an n-propyl group, an n-butyl group, an optionally at least mono-substituted phenyl group, an optionally at least mono-substituted benzyl group, an optionally at least mono-substituted naphthyl group, which may be bonded via a C₁₋₃-alkylene group, an optionally at least mono-substituted thienyl group, an optionally at least mono-substituted 2,1,3-Benzothiadiazole group, an optionally at least mono-substituted Benzo[b]thiophenyl group, an optionally at least mono-substituted Imidazo[2.1-b]thiazole group, an optionally at least mono-substituted 1H-pyrazole group or a 7,7-Dimethyl-2-oxo-bicyclo-[2.2.1]-hept-1-yl group, most preferably R¹¹ represents a methyl group, an ethyl group, an n-propyl group, an n-butyl group, an optionally at least mono-substituted phenyl group, an optionally at least mono-substituted benzyl group, an optionally at least mono-substituted naphthyl group, which may be bonded via a C₁₋₃-alkylene group, an optionally at least mono-substituted thienyl group, an optionally at least mono-substituted 2,1,3-Benzothiadiazole group, an optionally at least mono-substituted Benzo[b]thiophenyl group, an optionally at least mono-substituted Imidazo[2.1-b]thiazole group, an optionally at least mono-substituted 1H-pyrazole group or a 7,7-Dimethyl-2-oxo-bicyclo-[2.2.1]-hept-1-yl group, wherein said substituents, if present, are identical or different and selected from the group consisting of a fluorine atom, a chlorine atom, a bromine atom, a methyl group, a formyl group, a phenyl group, a phenoxy group, a phenoxy group substituted with bromine in the 4-position and a methylsulfonyl group, and the residues R¹—R¹⁰, R¹² and R¹³ have the meaning given above optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, a corresponding salt thereof, or a corresponding solvate thereof.

Also preferred-are compounds-of general formula I given above, wherein R¹² represents a linear or branched, saturated or unsaturated, optionally at least mono-substituted C₁₋₁₀-aliphatic group, a saturated or unsaturated, optionally-at least mono-substituted, optionally at least one heteroatom as ring member containing C₃₋₈-cycloaliphatic group, which may be condensed with a mono- or polycyclic ring-system and/or which may be bonded via a linear or bridged C₁₋₁₀-alkylene group and/or which may be bridged by a linear or branched C₁₋₅-alkylene group, or an optionally at least mono-substituted 5- or 6-membered aryl or heteroaryl group, which may be condensed with a mono- or polycyclic ring system and/or which may be bonded via a linear or branched C₁₋₁₀-alkylene group, preferably R¹² represents a linear or branched C₁₋₁₀-alkyl group, a saturated or unsaturated, optionally at least mono-substituted, optionally at least one heteroatom as ring member containing C₅₋₆-cycloaliphatic group, which may be condensed with a mono- or polycyclic ring-system and/or which may be bonded via a linear or bridged C₁₋₃-alkylene group and/or which may be bridged by a linear or branched C₁₋₃-alkylene group, or an optionally at least mono-substituted 5- or 6-membered aryl or heteroaryl group, which may be condensed with a mono- or polycyclic ring system and/or which may be bonded via a linear or branched C₁₋₃-alkylene group, more preferably R¹² represents a methyl group, an ethyl group, an n-propyl group, an n-butyl group, an optionally at least mono-substituted phenyl group, an optionally at least mono-substituted benzyl group, an optionally at least mono-substituted naphthyl group, which may be bonded via a C₁₋₃-alkylene group, an optionally at least mono-substituted thienyl group, an optionally at least mono-substituted 2,1,3-Benzothiadiazole group, an optionally at least mono-substituted Benzo[b]thiophenyl group, an optionally at least mono-substituted Imidazo[2.1-b]thiazole group, an optionally at least mono-substituted 1H-pyrazole group or a 7,7-Dimethyl-2-oxo-bicyclo-[2.2.1]-hept-1-yl group, most preferably R¹² represents a methyl group, an ethyl group, an n-propyl group, an n-butyl group, an optionally at least mono-substituted phenyl group, an optionally at least mono-substituted benzyl group, an optionally at least mono-substituted naphthyl group, which may be bonded via a C₁₋₃-alkylene group, an optionally at least mono-substituted thienyl group, an optionally at least mono-substituted 2,1,3-Benzothiadiazole group, an optionally at least mono-substituted Benzo[b]thiophenyl group, an optionally at least mono-substituted Imidazo[2.1-b]thiazole group, an optionally at least mono-substituted 1H-pyrazole group or a 7,7-Dimethyl-2-oxo-bicyclo-[2.2.1]-hept-1-yl group, wherein said substituents, if present, are identical or different and selected from the group consisting of a fluorine atom, a chlorine atom, a bromine atom, a methyl group, a formyl group, a phenyl group, a phenoxy group, a phenoxy group substituted with bromine in the 4-position and a methylsulfonyl group, and residues R¹—R¹¹ and R¹³ have the meaning given above, optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, a corresponding salt thereof, or a corresponding solvate thereof.

Also preferred are compounds of general formula I, wherein R¹³ represents a linear or branched, saturated or unsaturated, optionally at least mono-substituted C₁₋₁₀-aliphatic group, a saturated or unsaturated, optionally at least mono-substituted, optionally at least one heteroatom as ring member containing C₃₋₈-cycloaliphatic group, which may be condensed with a mono- or polycyclic ring-system and/or which may be bonded via a linear or bridged C₁₋₁₀-alkylene group and/or which may be bridged by a linear or branched C₁₋₅-alkylene group, or an optionally at least mono-substituted 5- or 6-membered aryl or heteroaryl group, which may be condensed with a mono- or polycyclic ring system and/or which may be bonded via a linear or branched C₁₋₁₀-alkylene group, preferably R¹³ represents a linear or branched C₁₋₁₀-alkyl group, a saturated or unsaturated, optionally at least mono-substituted, optionally at least one heteroatom as ring member containing C₅₋₆-cycloaliphatic group, which may be condensed with a mono- or polycyclic ring-system and/or which may be bonded via a linear or bridged C₁₋₃-alkylene group and/or which may be bridged by a linear or branched C₁₋₃-alkylene group, or an optionally at least mono-substituted 5- or 6-membered aryl or heteroaryl group, which may be condensed with a mono- or polycyclic ring system and/or which may be bonded via a linear or branched C₁₋₃-alkylene group, more preferably R¹³ represents a methyl group, an ethyl group, an n-propyl group, an n-butyl group, an optionally at least mono-substituted phenyl group, an optionally at least mono-substituted benzyl group, an optionally at least mono-substituted naphthyl group, which may be bonded via a C₁₋₃-alkylene group, an optionally at least mono-substituted thienyl group, an optionally at least mono-substituted 2,1,3-Benzothiadiazole group, an optionally at least mono-substituted Benzo[b]thiophenyl group, an optionally at least mono-substituted Imidazo[2.1-b]thiazole group, an optionally at least mono-substituted 1H-pyrazole group of a 7,7-Dimethyl-2-oxo-bicyclo-[2.2.1]-hept-1-yl group, most preferably R¹³ represents a methyl group, an ethyl group, an n-propyl group, an n-butyl group, an optionally at least mono-substituted phenyl group, an optionally at least mono-substituted benzyl group, an optionally at least mono-substituted naphthyl group, which may be bonded via a C₁₋₃-alkylene group, an optionally at least mono-substituted thienyl group, an optionally at least mono-substituted 2,1,3-Benzothiadiazole group, an optionally at least mono-substituted Benzo[b]thiophenyl group, an optionally at least mono-substituted Imidazo[2.1-b]thiazole group, an optionally at least mono-substituted 1H-pyrazole group or a 7,7-Dimethyl-2-oxo-bicyclo-[2.2.1]-hept-1-yl group, wherein said substituents, if present, are identical or different and selected from the group consisting of a fluorine atom, a chlorine atom, a bromine atom, a methyl group, a formyl group, a phenyl group, a phenoxy group, a phenoxy group substituted with bromine in the 4-position and a methylsulfonyl group, and R¹—R¹² have the meaning given above optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, a corresponding salt thereof, or a corresponding solvate thereof.

Particular preference is also given to compounds of general formula I, 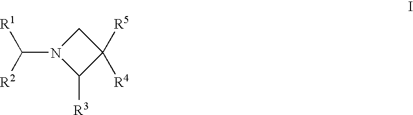
wherein

R¹ represents a phenyl group, said phenyl group being unsubstituted or substituted with 1, 2, 3, 4 or 5 substituents independently selected from the group consisting of F, Cl, Br, linear or branched C₁₋₆ alkyl, linear or branched C₁₋₆ alcoxy, —(C═O)H, —OH, —CF₃, —OCF₃, —CN and —COOH,

R² represents a phenyl group, said phenyl group being unsubstituted or substituted with 1, 2, 3, 4 or 5 substituents independently selected from the group consisting of F, Cl, Br, linear or branched C₁₋₆ alkyl, linear or branched C₁₋₆ alcoxy, —(C═O)—H, —OH, —CF₃, —OCF₃, —CN and —COOH,

R³ represents a linear or branched, unsubstituted C₁₋₆ alkyl group,

R⁴ represents a hydrogen atom, —CN, —COOH, a linear or branched, unsubstituted C₁₋₆ alkyl group, or a phenyl group, said phenyl group being unsubstituted or substituted with 1, 2, 3, 4 or 5 substituents independently selected from the group consisting of F, Cl, Br, linear or branched C₁₋₆ alkyl, linear or branched C₁₋₆ alcoxy, —(C═O)—H, —OH, —CF₃, —OCF₃, —CN and —COOH,

R⁵ represents an —O—SO₂—R⁶-moiety, an —NH—CO—R⁷-moiety, an —NH₂-moiety, an —NH—SO₂—R⁸ moiety, or an —NR⁹—SO₂—R¹⁰-moiety or an —O—C(═O)—R¹¹-moiety,

R⁶ represents a phenyl group, said phenyl group being unsubstituted or substituted with 1, 2, 3, 4 or 5 substituents independently selected from the group consisting of F, Cl, Br, linear or branched C₁₋₆ alkyl, linear or branched C₁₋₆ alcoxy, —(C═O)—H, —OH, —CF₃, —OCF₃, —CN and —COOH,

R⁷ represents a linear or branched C₁₋₅ alkyl group, said alkyl group being unsubstituted or substituted with 1, 2, 3, 4 or 5 substituents independently selected from the group consisting of F, Cl, Br, —OH, —SH, linear or branched C₁₋₆ alcoxy, —CF₃ and —OCF₃; a cyclopentyl or cyclohexyl group, said cyclopentyl or cyclohexyl group being unsubstituted or substituted with 1, 2, 3, 4 or 5 substituents independently selected from the group consisting of F, Cl, Br, —OH, —SH, linear or branched C₁₋₆ alcoxy, —CF₃ and —OCF₃; or a phenyl group, said phenyl group being unsubstituted or substituted with 1, 2, 3, 4 or 5 substituents independently selected from the group consisting of F, Cl, Br, linear or branched C₁₋₆ alkyl, linear or branched C₁₋₆ alcoxy, —(C═O)—H, —OH, —CF₃, —OCF₃, —CN and —COOH,

R⁸ represents a linear or branched C₁₋₅ alkyl group; an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alcoxy, phenyl (optionally substituted with 1, 2, 3, 4 or 5 substituents independently selected from the group consisting of F, Cl, Br, OH, linear or branched C₁₋₆ alkyl and linear or branched C₁₋₆-alcoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alcoxy), —SO₂—CH₃, —(C═O)H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alcoxy, —(C═O)—H, —OH, —CF₃, ═O, —OCF₃, —CN and —COOH;

R⁹ represents an —SO₂—R¹²-moiety; a —C═O)—R¹³-moiety; a linear or branched C₁₋₆ alkyl radical; or a phenyl radical, said phenyl radical being unsubstituted or substituted with 1, 2, 3, 4 or 5 substituents independently selected from the group consisting of F, Cl, Br, linear or branched C₁₋₆ alkyl, linear or branched C₁₋₆ alcoxy, —(C═O)—H, —OH, —CF₃, —OCF₃, —CN and —COOH and/or said-phenyl radical being optionally bonded via a C₁₋₃-alkylene bridge,

R¹⁰ represents a linear or branched C₁₋₅ alkyl group; an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alcoxy, phenyl (optionally substituted with 1, 2, 3, 4 or 5 substituents independently selected from the group consisting of F, Cl, Br, OH, linear or branched C₁₋₆ alkyl and linear or branched C₁₋₆-alcoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alcoxy), —SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alcoxy, —(C═O)—H, —OH, —CF₃, ═O, —OCF₃, —CN and —COOH;

R¹¹ represents a linear or branched C₁₋₅ alkyl group; an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alcoxy, phenyl (optionally substituted with 1, 2, 3, 4 or 5 substituents independently selected from the group consisting of F, Cl, Br, OH, linear or branched C₁₋₆ alkyl and linear or branched C₁₋₆-alcoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alcoxy), —SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alcoxy, —(C═O)—H, —OH, —CF₃, ═O, —OCF₃, —CN and —COOH;

R¹² represents a linear or branched C₁₋₅ alkyl group, an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alcoxy, phenyl (optionally substituted with 1, 2, 3, 4 or 5 substituents independently selected from the group consisting of F, Cl, Br, OH, linear or branched C₁₋₆ alkyl and linear or branched C₁₋₆-alcoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alcoxy), —SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alcoxy, —(C═O)—H, —OH, —CF₃, ═O, —OCF₃, —CN and —COOH;

R¹³ represents a linear or branched C₁₋₅ alkyl group, an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alcoxy, phenyl (optionally substituted with 1, 2, 3, 4 or 5 substituents independently selected from the group consisting of F, Cl, Br, OH, linear or branched C₁₋₆ alkyl and linear or branched C₁₋₆-alcoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alcoxy), —SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alcoxy, —(C═O)—H, —OH, —CF₃, ═O, —OCF₃, —CN and —COOH;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, a corresponding salt thereof, or a corresponding solvate thereof,

especially to compounds of the following formula Ib, 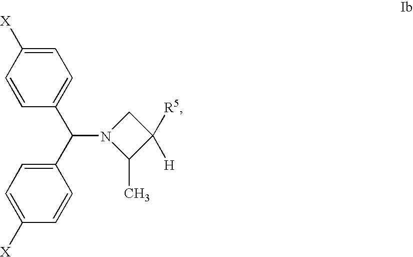
wherein

wherein X represents a halogen atom, preferably a chlorine atom,

R⁵ represents an —O—SO₂—R⁶-moiety, an —NH—CO—R⁷-moiety, an —NH₂-moiety, an —NH—SO₂—R⁸ moiety, or an —NR⁹—SO₂—R¹⁰-moiety or an —O—C(═O)—R¹¹-moiety,

R⁶ represents a phenyl group, said phenyl group being unsubstituted or substituted with 1, 2, 3, 4 or 5 substituents independently selected from the group consisting of F, Cl, Br, linear or branched C₁₋₆ alkyl, linear or branched C₁₋₆ alcoxy, —(C═O)—H, —OH, —CF₃, —OCF₃, —CN and —COOH,

R⁷ represents a linear or branched C₁₋₅ alkyl group, said alkyl group being unsubstituted or substituted with 1, 2, 3, 4 or 5 substituents independently selected from the group consisting of F, Cl, Br, —OH, —SH, linear or branched C₁₋₆ alcoxy, —CF₃ and —OCF₃; a cyclopentyl or cyclohexyl group, said cyclopentyl or cyclohexyl group being unsubstituted or substituted with 1, 2, 3, 4 or 5 substituents independently selected from the group consisting of F, Cl, Br, —OH, —SH, linear or branched C₁₋₆ alcoxy, —CF₃ and —OCF₃; or a phenyl group, said phenyl group being unsubstituted or substituted with 1, 2, 3, 4 or 5 substituents independently selected from the group consisting of F, Cl, Br, linear or branched C₁₋₆ alkyl, linear or branched C₁₋₆ alcoxy, —(C═O)—H, —OH, —CF₃, —OCF₃, —CN and —COOH,

R⁸ represents a linear or branched C₁₋₅ alkyl group; an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alcoxy, phenyl (optionally substituted with 1, 2, 3, 4 or 5 substituents independently selected from the group consisting of F, Cl, Br, OH, linear or branched C₁₋₆ alkyl and linear or branched C₁₋₆-alcoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alcoxy), —SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alcoxy, —(C═O)—H, —OH, —CF₃, ═O, —OCF₃, —CN and —COOH;

R⁹ represents an —SO₂—R¹²-moiety; a —(C═O)—R¹³-moiety; a linear or branched C₁₋₆ alkyl radical; or a phenyl radical, said phenyl radical being unsubstituted or substituted with 1, 2, 3, 4 or 5 substituents independently selected from the group consisting of F, Cl, Br, linear or branched C₁₋₆ alkyl, linear or branched C₁₋₆ alcoxy, —(C═O)—H, —OH, —CF₃, —OCF₃, —CN and —COOH and/or said phenyl radical being optionally bonded via a C₁₋₃-alkylene bridge,

R¹⁰ represents a linear or branched C₁₋₅ alkyl group; an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alcoxy, phenyl (optionally substituted with 1, 2, 3, 4 or 5 substituents independently selected from the group consisting of F, Cl, Br, OH, linear or branched C₁₋₆ alkyl and linear or branched C₁₋₆-alcoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alcoxy), —SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alcoxy, —(C═O)—H, —OH, —CF₃, ═O, —OCF₃, —CN and —COOH;

R¹¹ represents a linear or branched C₁₋₅ alkyl group; an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alcoxy, phenyl (optionally substituted with 1, 2, 3, 4 or 5 substituents independently selected from the group consisting of F, Cl, Br, OH, linear or branched C₁₋₆ alkyl and linear or branched C₁₋₆-alcoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alcoxy), —SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alcoxy, —(C═O)—H, —OH, —CF₃, ═O, —OCF₃, —CN and —COOH;

R¹² represents a linear or branched C₁₋₅ alkyl group, an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alcoxy, phenyl (optionally substituted with 1, 2, 3, 4 or 5 substituents independently selected from the group consisting of F, Cl, Br, OH, linear or branched C₁₋₆ alkyl and linear or branched C₁₋₆-alcoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alcoxy), —SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alcoxy, —(C═O)—H, —OH, —CF₃, ═O, —OCF₃, —CN and —COOH;

R¹³ represents a linear or branched C₁₋₅ alkyl group, an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alcoxy, phenyl (optionally substituted with 1, 2, 3, 4 or 5 substituents independently selected from the group consisting of F, Cl, Br, OH, linear or branched C₁₋₆ alkyl and linear or branched C₁₋₆-alcoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alcoxy), —SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alcoxy, —(C═O)—H, —OH, —CF₃, ═O, —OCF₃, —CN and —COOH;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, a corresponding salt thereof, or a corresponding solvate thereof.

Particularly preferred are compounds of general formula I, 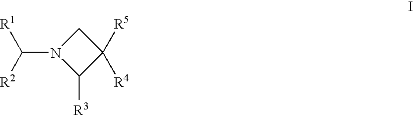
wherein

R¹ represents a phenyl group, which is mono-substituted with a halogen atom, preferably a chlorine atom, in the 4-position of the phenyl ring,

R² represents a phenyl group, which is mono-substituted with a halogen atom, preferably a chlorine atom, in the 4-position of the phenyl ring,

R³ represents a linear or branched, unsubstituted C₁₋₆ alkyl group, preferably a methyl group,

R⁴ represents a hydrogen atom,

R⁵ represents an —O—SO₂—R⁶-moiety, an —NH—CO—R⁷-moiety, an —NH₂-moiety, an —NH—SO₂—R⁸ moiety, or an —NR⁹—SO₂—R¹⁰-moiety

R⁶ represents a phenyl ring, which is optionally substituted with one or more halogen atoms, preferably one or more fluorine and/or one or more chlorine atoms,

R⁷ represents a linear or branched C₁₋₅ alkyl group, a linear or branched C₁₋₅ alkyl group, which is at least partially fluorinated, a C₃₋₈ cycloalkyl group, or a phenyl group, which is optionally substituted with one or more halogen atoms, preferably one or more fluorine atoms,

R⁸ represents a linear or branched C₁₋₅ alkyl group,

a phenyl group, which is optionally substituted with one or more substituents independently selected from the group consisting of a fluorine atom, a chlorine atom an unsubstituted phenyl group, a formyl group, a methylsulfonyl group, a benzyl group and a phenoxy group, which is optionally mono-substituted by a bromine atom in its 4-position,

a naphthyl group, which may be bonded via a methylene or ethylene group,

a Benzo[b]thiophene group, which is optionally substituted with one or more methyl groups and/or one or more chlorine atoms,

a pyrazole group, which is optionally substituted with one or more substituents independently selected from the group consisting of a methyl group, an ethyl group and a phenyl group,

an imidazo[2,1-b]thiazole group, which is optionally substituted with one or more chlorine atoms,

a thienyl group, a furyl group, a 2,1,3-Benzothiadiazole group, a 7,7-Dimethyl-2-oxo-bicyclo-[2.2.1]-hept-1-yl group, or a benzyl group,

R⁹ represents a C₁₋₅ alkyl group, preferably a methyl group, a phenyl group, which is optionally substituted with one or more fluorine atoms and/or one or more chlorine atoms, a benzyl group, wherein the ring is optionally substituted with one or more fluorine atoms and/or one or more chlorine atoms, or a —SO₂—R¹²-moiety,

R¹⁰ represents a phenyl group, which is optionally substituted with one or more fluorine atoms and/or one or more chlorine atoms,

R¹² represents a C₁₋₅ alkyl group, preferably a methyl group, or a phenyl group, which is optionally substituted with one or more fluorine atoms and/or one or more chlorine atoms,

optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, a corresponding salt thereof, or a corresponding solvate thereof.

Most particularly preferred are substituted azetidine compounds of general formula I given above selected from the group consisting of
[1] 4-Fluoro-benzenesulfonic acid 1-[trans-bis-(4-chloro-phenyl)-methyl]-2-methyl-azetidin-3-yl ester,
[2] N-{(2S,3R)-1-[Bis-(4-chloro-phenyl)-methyl]-2-methyl-azetidin-3-yl}-2,2,2-trifluoro-acetamide,
[3] (2S,3R)-1-[Bis-(4-chloro-phenyl)-methyl]-2-methyl-azetidin-3-ylamine,
[4] Hexanoic acid {1-[bis-(4-chloro-phenyl)-methyl]-2-methyl-azetidin-3-yl}-amide,
[5] N-{(2S,3R)-1-[Bis-(4-chloro-phenyl)-methyl]-2-methyl-azetidin-3-yl}-4-fluoro-benzenesulfonamide,
[6] Thiophene-2-sulfonic acid {(2S,3R)-1-[bis-(4-chloro-phenyl)-methyl]-2-methyl-azetidin-3-yl}-amide,
[7] Cyclohexanecarboxylic acid {(2S,3R)-1-[bis-(4-chloro-phenyl)-methyl]-2-methyl-azetidin-3-yl}-amide,
[8] Butane-1-sulfonic acid {(2S,3R)-1-[bis-(4-chloro-phenyl)-methyl]-2-methyl-azetidin-3-yl}-amide,
[9] N-{(2S,3R)-1-[Bis-(4-chloro-phenyl)-methyl]-2-methyl-azetidin-3-yl}-3,5-difluoro-benzamide,
[10] Naphthalene-2-sulfonic acid {trans-1-[bis-(4-chloro-phenyl)-methyl]-2-methyl-azetidin-3-yl}-amide,
[11] Biphenyl-4-sulfonic acid {trans-1-[bis-(4-chloro-phenyl)-methyl]-2-methyl-azetidin-3-yl}-amide,
[12] 4-Acetyl-N-{trans-1-[bis-(4-chloro-phenyl)-methyl]-2-methyl-azetidin-3-yl}-benzenesulfonamide,
[13] N-{trans-1-[Bis-(4-chloro-phenyl)-methyl]-2-methyl-azetidin-3-yl}-4-(4-bromo-phenoxy)-benzenesulfonamide,
[14] N-{trans-1-[Bis-(4-chloro-phenyl)-methyl]-2-methyl-azetidin-3-yl}-4-methylsulfonyl-benzenesulfonamide,
[15] 2,1,3-Benzothiadiazole-4-sulfonic acid {trans-1-[bis-(4-chloro-phenyl)-methyl]-2-methyl-azetidin-3-yl}-amide,
[16] 5-Chloro-3-methyl-benzo[b]thiophene-2-sulfonic acid {trans-1-[bis-(4-chloro-phenyl)-methyl]-2-methyl-azetidin-3-yl}-amide,
[17] 6-Chloro-imidazo[2,1-b]thiazole-5-sulfonic acid {trans-1-[bis-(4-chloro-phenyl)-methyl]-2-methyl-azetidin-3-yl}-amide,
[18] N-{trans-1-[Bis-(4-chloro-phenyl)-methyl]-2-methyl-azetidin-3-yl}-3,5-dichloro-benzenesulfonamide,
[19] 2-Naphthalene-1-yl-ethanesulfonic acid {trans-1-[bis-(4-chloro-phenyl)-methyl]-2-methyl-azetidin-3-yl}-amide,
[20] N-{trans-1-[Bis-(4-chloro-phenyl)-methyl]-2-methyl-azetidin-3-yl}-phenyl-methylsulfonamide,
[21] N-{trans-1-[Bis-(4-chloro-phenyl)-methyl]-2-methyl-azetidin-3-yl}-(7,7-dimethyl-2-oxo-bicyclo[2.2.1]hept-1-yl)-methylsulfonamide,
[22] Naphthalene-1-sulfonic acid {trans-1-[bis-(4-chloro-phenyl)-methyl]-2-methyl-azetidin-3-yl}-amide,
[23] N-{trans-1-[Bis-(4-chloro-phenyl)-methyl]-2-methyl-azetidin-3-yl}-4-phenoxy-benzenesulfonamide,
[24] 1,3,5-Trimethyl-1H-pyrazole-4-sulfonic acid{trans-1-[bis-(4-chloro-phenyl)-methyl]-2-methyl-azetidin-3-yl}-amide,
[25] Benzo[b]thiophene-3-sulfonic acid {trans-1-[bis-(4-chloro-phenyl)-methyl]-2-methyl-azetidin-3-yl}-amide,
[26] 5-Methyl-1-phenyl-1H-pyrazole-4-sulfonic acid {trans-1-[bis-(4-chloro-phenyl)-methyl]-2-methyl-azetidin-3-yl}-amide,
[27] N-{trans-1-[Bis-(4-chloro-phenyl)-methyl]-2-methyl-azetidin-3-yl}-N-methyl-4-fluoro-benzenesulfonamide,
[28] N-{trans-1-[Bis-(4-chloro-phenyl)-methyl]-2-methyl-azetidin-3-yl}-N-(4-fluoro-benzyl)-4-fluoro-benzenesulfonamide,
[29] N-{trans-1-[Bis-(4-chloro-phenyl)-methyl]-2-methyl-azetidin-3-yl}-N-propyl-4-fluoro-benzenesulfonamide,
[30] N-{trans-1-[Bis-(4-chloro-phenyl)-methyl]-2-methyl-azetidin-3-yl}-N-(methylsulphonyl)-4-fluoro-benzenesulfonamide,
[31] N-{trans-1-[Bis-(4-chloro-phenyl)-methyl]-2-methyl-azetidin-3-yl}-N-bis (4-fluoro-benzenesulfonamide),
[32] N-{(trans-1-[Bis-(4-chloro-phenyl)-methyl]-2-methyl-azetidin-3-yl}-4-fluoro-benzenesulfonamide, and
[33] N-{(2R,3S)-1-[Bis-(4-chloro-phenyl)-methyl]-2-methyl-azetidin-3-yl}-4-fluoro-benzenesulfonamide;
optionally in form of a corresponding N-oxide, a corresponding salt or a corresponding solvate.

In another aspect the present invention provides a process for the preparation of substituted azetidine compounds of general formula I given above and corresponding stereoisomers thereof, according to which at least one compound of general formula II,

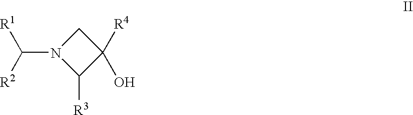

wherein R¹ to R⁴ have the meaning given above, is reacted with at least one compound of general formula X¹—SO₂—R⁶ or X²—CO—R¹¹, wherein R⁶ and R¹¹ have the meaning given above and X¹ and X² represent suitable leaving groups, in a suitable reaction medium, optionally in the presence of at least one base, to yield at least one compound of general formula I given above, wherein R⁵ represents an —O—SO₂—R⁶-moiety or an —O—CO—R¹¹-moiety, and optionally purifying and/or optionally isolating said compound(s),

and optionally at least one of these afore mentioned compounds, wherein R⁵ represents an —O—SO₂—R⁶ or an —O—CO—R¹¹ moiety, is reacted with ammonia, to yield a compound of general formula I, wherein R⁵ represents an —NH₂-moiety, and optionally purifying and/or optionally isolating said compound(s),

and optionally at least one of these afore mentioned compounds, wherein R⁵ represents an —NH₂-moiety, is reacted with at least one compound of general formula X³—COR⁷, X⁴—SO₂—R⁸ or X⁵—SO₂—R¹⁰, wherein R⁷, R⁸ and R¹⁰ have the meaning given above and X³, X⁴ and X⁵ are suitable leaving groups, in a reaction medium, optionally in the presence of at least one base, to yield a compound of general formula I given above, wherein R⁵ represents an —NH—CO—R⁷-moiety, an —NH—SO₂—R⁸-moiety or an —NR⁹—SO₂—R¹⁰-moiety with R⁹ representing a hydrogen atom, and optionally purifying and/or optionally isolating said compound(s),

and optionally at least one compound of general formula I, wherein R⁵ represents an —NR⁹—SO₂—R¹⁰-moiety with R⁹ representing a hydrogen atom is reacted with at least one compound of general formula X⁶—R⁹, wherein R⁹ has the meaning given above except for a hydrogen atom and X⁶ is a leaving group, to yield at least one compound of general formula I given above, wherein R⁵ represents an —NR⁹—SO₂—R¹⁰-moiety, and optionally purifying and/or optionally isolating said compound(s),

or, according to which at least one compound of general formula III,

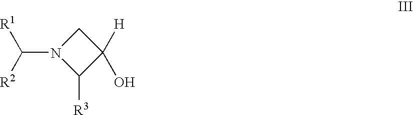

wherein R⁴ represents a hydrogen atom and R¹—R³ have the meaning given above, is oxidized to yield at least one compound of general formula IV,

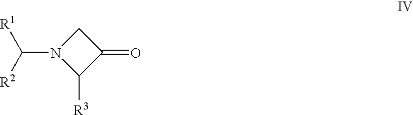

wherein R¹—R³ have the meaning given above, which is optionally purified and/or optionally isolated, and reacted with at least one compound of general formula R^{5a}H, wherein R^{5a} represents an —NH₂-moiety or an —NHR⁹-moiety, wherein R⁹ has the meaning given above, the resulting compound is optionally purified and/or optionally isolated and optionally reacted with at least one compound of general formula X³—CO—R⁷, X⁴—SO₂—R⁸ or X⁵—SO₂—R¹⁰, wherein R⁷, R⁸ and R¹⁰ have the meaning given above and X³, X⁴ and X⁵ represent a leaving group, in a reaction medium, optionally in the presence of at least one base, to yield a compound of general formula I given above, wherein R⁵ represents an —NH₂-moiety, an —NH—CO—R⁷-moiety, an —NH—SO₂—R⁸-moiety, or an —NR⁹—SO₂—R¹⁰-moiety, which is optionally purified and/or isolated. Said inventive process is also illustrated in the following scheme 1:

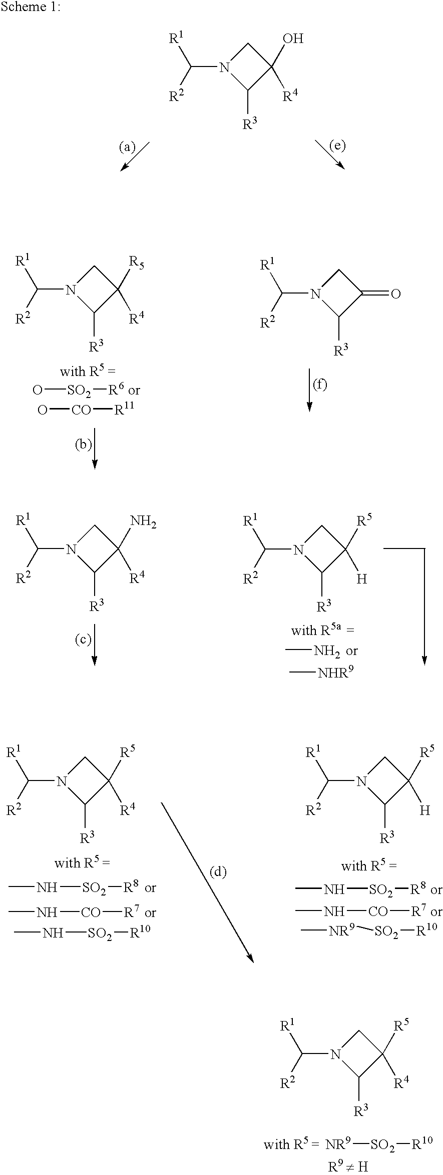

Preferably step (a) according to scheme 1 is carried out in one or more organic solvents as reaction medium. Suitable solvents include but are not limited to halogenated, preferably chlorinated, organic solvents such as dichloromethane or chloroforme and linear or cyclic ethers, such as tetrahydrofurane, 1,4-dioxane, or 1,1-dimethoxyethane. Reaction temperature as well as the duration of the reaction may vary over a broad range. The optimum reaction temperature and duration of reaction for a given reaction may be determined by conventional methods known to those skilled in the art.

Preferred reaction temperatures are 0-30° C., preferably 15-25° C. Suitable reaction times may vary from approximately 10 minutes to 3 days.

Suitable bases for use in step (a) include organic bases such as triethylamine or pyridine as well as inorganic bases such as sodium hydroxide or potassium hydroxide. Mixtures of one or more organic bases and/or one or more inorganic bases may also be used.

The leaving groups X¹ and X² may be of any type known to those skilled in the art for this type of reaction. Preferably the leaving group is a halogen atom, more preferably a chlorine or bromine atom.

Suitable reaction media for the reaction with ammonia according to step (b) of scheme 1 include, for example, alcohols such as methanol, ethanol, isopropanol or mixtures of at least two of these alcohols. The ammonia is preferably added as a concentrated, preferably aqueous solution.

Reaction temperature, pressure as well as the duration of the reaction may vary over a broad range. The optimum conditions may be determined by conventional methods known to those skilled in the art. Preferred reaction temperatures range from ambient temperature, i.e. approximately 15-25° C. to the boiling point of the reaction medium. Suitable reaction times may vary, for example from approximately 10 minutes to 3 days. Obviously the reaction may also be carried out in a reactor at elevated temperatures and pressure. Preferably compounds of general formula I, wherein R⁵ represents a —O—SO₂—R⁶-moiety with R⁶ representing a methyl group, are used in step (b).

The reaction steps (c) and (d) according to scheme 1 may also be carried out under conventional conditions known to those skilled in the art. A suitable reaction medium used for these reaction steps is preferably comprises one or more organic solvents.

Suitable solvents include but are not limited to halogenated, preferably chlorinated, organic solvents such as dichloromethane or chloroforme and linear or cyclic ethers, such as tetrahydrofurane, 1,4-dioxane, or 1,1-dimethoxyethane. Reaction temperature as well as the duration of the reaction may vary over a broad range. The optimum conditions for a given reaction may be determined by conventional methods known to those skilled in the art. Preferred reaction temperatures are 0-30° C., preferably 15-25° C. Suitable reaction times vary, e.g. from approximately 10 minutes to 3 days.

Suitable bases that may be present during reaction steps (c) and (d) include organic bases such as triethylamine or pyridine as well as inorganic bases such as sodium hydroxide or potassium hydroxide. Mixtures of one or more organic bases and/or one or more inorganic bases may also be used.

The leaving groups X³, X⁴, X⁵ and X⁶ may be of any type known to those skilled in the art for this type of reaction. Preferably the leaving group is a halogen atom, more preferably a chlorine or bromine atom.

The preparation of compounds of general formula IV according to step (e) of scheme 1 may preferably be carried out according to the literature publications of Katritzky et al., J. Heterocycl. Chem., 1994, 271-275; P. R. Dave et al., J. Org. Chem., 1996, 61(16), 5453, Synlett. 1991, (11), 783-784 and Axenrod et al., Tetrahedron Lett., 1993, 6677-6680. The respective descriptions are hereby incorporated by reference and form part of the present disclosure.

The respective compounds of general formula R^{5a}H, wherein R^{5a} represents an —NH₂-moiety or an —NHR⁹-moiety, wherein R⁹ has the meaning given above, are also commercially available or may be prepared by conventional methods known to those skilled in the art.

Preferably step (f) according to scheme 1 is carried out in one or more organic solvents as reaction medium such as chlorinated, organic solvents like dichloromethane or chloroforme and preferably in the presence of sodium triacetoxyborohydride and acetic acid, or in the presence of H₂ and an alcohol such as methanol and/or ethanol as a reaction medium.

Reaction temperature as well as the duration of the reaction may vary over a broad range. The optimum reaction temperature and duration of reaction for a given reaction may be determined by conventional methods known to those skilled in the art. Preferred reaction temperatures are 0-30° C., preferably 15-25° C.

The substituted 3-azetidinol compounds of general formula III may be prepared according to scheme 2 given below:

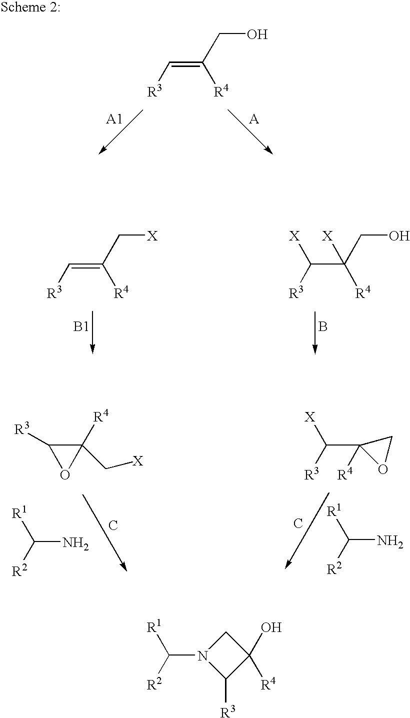

In Scheme 2 the substituents R¹—R⁴ have the same meaning as definied above and X represents a halogen atom, preferably a bromine or chlorine atom.

The substituted hydroxy-alkenylene compounds used as a starting material for reaction steps A and A1 according to scheme 2 are commercially available and/or may be prepared according to methods well known to those skilled in the art, e.g. by reduction of the corresponding carbonyl compound with a suitable reducing agent such as aluminium lithium hydride, sodium borohydride or catalysed hydrogenation as described, for example, in Catalysis Letters, 1999, 62(24), 175-177; Tetrahedron, 1984, 40(7), 1195-1198; J. Org. Chem., 1999, 64(7), 2582-2589; J. Am. Chem. Soc., 2001, 12168-12175; Synlett. 1763-65 (1999); Bull. Soc. Chim. France, 132(5-6), 522-30 (1995); Tetrahedron Lett., 43(49), pages 8893-8896 (2002) and J. Natural Products, 65(6), 902-908 (2002). The corresponding descriptions are hereby incorporated by reference and form part of the present disclosure.

The halogenenated alkenylene compounds used as starting material for reaction step B and B1 are commercially available and/or may be prepared from the corresponding hydroxy-alkenylene compounds using a suitable halogenation agent such as thionyl chloride, triphenyl phosphin/carbontetrachloride, bromine, hydrobromic acid, triphenyl phosphin/carbontetrabromide, phosphor tribromide and others described in the literature, for example, in Chem. Ber. 123(12), 2387-94, 1990; J. Am. Chem. Soc., 111(9), 3363-3368, 1989; J. Org. Chem., 63(25), 9565-68, 1998; Heterocycles 32(5), 965-73, 1991; Farmaco, 44(12), 1167-91, 1989; Synthesis, 8, 598-603, 1989; and J. Med. Chem., 33(3), 908-18, 1990. The corresponding descriptions are hereby incorporated by reference and form part of the present disclosure.

The reaction steps A, B and B1 may be carried out as described in Higgins et al., J. Heterocyclic Chem., 1971, 8, 1059-1062 and in U.S. Pat. No. 5,073,646. The corresponding parts of the descriptions are hereby incorporated by reference and form part of the present disclosure.

Reaction step A is preferably carried out in an organic solvent such as dichloromethane, chlorofom, carbontetrachloride or mixtures thereof as reaction medium.

Reaction temperature as well as the duration of the reaction may vary over a broad range. The optimum conditions for a given reaction may be determined by conventional methods known to those skilled in the art. Preferred reaction temperatures are from −10° C. to +30° C., preferably from 0° C. to 25° C., more preferably from 0° C. to 10° C. Suitable reaction times vary from a few minutes to several hours, preferably from 3 to 8 hours.

Reaction step B is preferably carried out by dissolving the educt in a suitable reaction medium, preferably a linear ether such as diethylether, or a cyclic ether, such as tetrahydrofuran, 1,4-dioxane or 1,1-dimethoxyethan, and in the presence of an aqueous solution of a base, preferably selected from the group consisting of an alkali metal such as lithium, sodium, or potassium, under vigorous stirring.

Reaction temperature as well as the duration of the reaction may vary over a broad range. The optimum reaction conditions for a given reaction may be determined by conventional methods known to those skilled in the art. Preferred reaction temperatures range from 10° C. to the boiling point of the reaction mixture, preferably from 15° C. to 25° C. Suitable reaction times vary from a few minutes to several hours, preferably from 3 to 8 hours

Reaction step B1 is preferably carried out in the presence of an oxidizing agent such as peracetic acid, m-chloro perbenzoic acid, N-Bromsuccinimide or the like.

The 3-azetidinol compound is obtained by carrying out reaction step (c) according to the methods described in the literature publications of M. E. Jung, J. Org. Chem., 1991, 56(24), 6729-6730; V. R. Gaertner, J. Org. Chem., 1967, 32, 2972, Katrizky et al., J. Heterocycl. Chem. 1994, 271-275, P. R. Dave et al, J. Org. Chem., 1996, 61(16), 5453 and U.S. Pat. No. 5,073,646. The corresponding descriptions are incorporated by reference and form part of the present disclosure.

The corresponding amines of general formula C(H)(R¹)(R²)—NH₂ are commercially available and/or may be obtained according to scheme 3 given below:

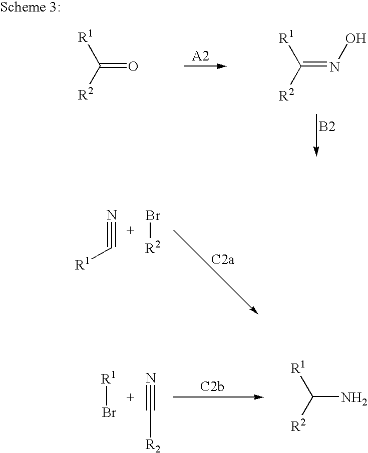

The reaction steps A2, B2, C2a and C2b may be carried out according to conventional methods well known to those skilled in the art. Step A2 is for example described in Hajipour et al., Synth. Commun. 1999, 29(10), 1697-1701, A. Sasse et al., Arch. Pharm., 2001, 334(2), 45-52. Step B2 is for example described in the publication of Fernandez et al., Synthesis 2001, (2), 239-242; Baruah et al., Synlett, 1999, (4), 409-410. Steps C2a and C2b are for example disclosed in the publications of M. Grisar et al., J. Med. Chem., 1973, 885 or Dejaegher et al., Synlett. 2002, 113-115. The respective descriptions are hereby incorporated by reference and form part of the present disclosure.

Another aspect the present invention is a process for the preparation of substituted azetidine compounds of general formula I given above and corresponding stereoisomers thereof, according to which at least one compound of general formula V

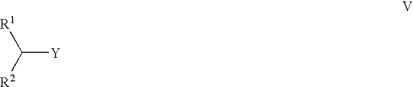

wherein R¹ and R² have the meaning given above and Y represents a halogen atom, preferably a chlorine atom or a bromine atom, is reacted with at least one compound of general formula VI,

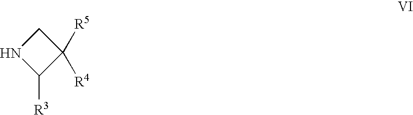

optionally in form of a salt, wherein R³, R⁴ and R⁵ have the meaning given above, in a suitable reaction medium, optionally in the presence of a base, and the resulting azetidine compound(s) is/are optionally purified and/or optionally isolated.

The compounds of general formula V may be obtained according to conventional methods skilled in the art. Some exemplary methods are illustrated in scheme 4 given below:

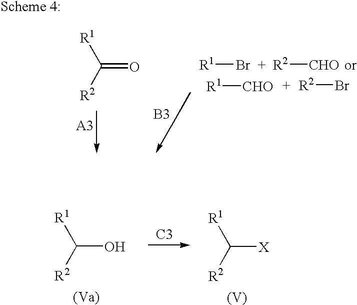

The compounds of general formula (Va) may, for example, be obtained according to step A3 of scheme 4 by reduction of a corresponding ketone compound with a suitable reducing agent such as sodium borohydride in a suitable reaction medium such as alcohol, preferably methanol, whereby the reaction temperature is preferably kept in the range of 0° C. and the boling point of the reaction medium.

The compounds of general formula (Va) may, for example, also be obtained according to step B3 of scheme 4 by a Grignard reaction in a suitable inert reaction medium such as diethylether or tetrahydrofuran, whereby the reation temperature is preferably kept in the range of 0° C. and the boiling point of the reaction medium.

The corresponding starting materials for the reactions according to steps A3 and B3 of scheme 4 are commercially available and/or may be prepared by methods well known to those skilled in the art.

Reaction step C3 of scheme 4, in which X represents a halogen atom, preferably a chlorine or bromine atom, may be carried out using a suitable halogenating agent such as hydrobromic acid, thionyl bromide, thionyl chloride, acetyl bromide, in a suitable reaction medium such as acetic acid, benzene, toluene, dichloromethane or chloroform, whereby the reaction temperature is preferably kept in the range of 0° C. to the boling point of the reaction medium.

The reaction of at least one compound of general formula V given above with at least one compound of general formula VI may preferably be carried out in an inert reaction medium, whereby acetonitrile, tetrahydrofuran or mixtures thereof are preferred. Suitable bases include organic bases such as triethylamine and inorganic bases such as carbonates of alkali metals, preferably potassium carbonate, or potassium iodide. The reaction temperature is preferably in the range of ambient temperature to the boling point of the reaction medium. The reaction times may vary over a broad range.

The compounds of general formula VI may be prepared from the corresponding substituted benzhydrylazetidine compounds of general formula VII,

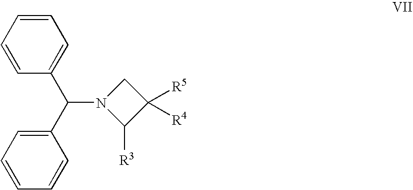

by hydrogenolysis, preferably in the presence of palladium metal on carbon powder, in a reaction medium such as an alcohol like methanol. Preferably said hydrogenolysis is carried out at ambient temperature, i.e. approximately 15-25° C. After the hydrogenolysis is completed, compounds of general formula VI are preferably isolated in form of a corresponding salt such as a hydrochloride or hydrobromide salt.

The compounds of general formula VII may be prepared according to the methods described, for example, in U.S. Pat. No. 5,073,646 and J. Frigola, J. Med. Chem., 1993, 36, 801-810. The compounds of general formula VI are usually obtained in form of a mixture of diastereoisomers. The respective enantiomers may be obtained by racemic resolution with HPLC using chiral columns or by crystallization with chiral derivatives obtained from the reaction of the corresponding racemate with chiral agents as described in the publication of J. Frigola, J. Med. Chem. 1994, 37, 4195-4210 and J. Frigola, J. Med. Chem., 1995, 38, 1203-1215. The corresponding enantiomers may also be obtained by asymmetric epoxidation in a corresponding reaction as described in scheme 2, which is carried out according to the method described in the publication of Sharpless et al., J. Am. Chem. Soc., 1980, 102, 5974-5976; J. A. Marshall et al., Org. Lett., 2000 2(18), 2897-2900 using tert.-Butyl hydroperoxide in the presence of titanium isopropoxide and diethyl or diisopropyl tartrate as oxidant.

The corresponding descriptions of the afore mentioned literature publications are hereby incorporated by reference and are part of the present disclosure.

During the processes described above the protection of sensitive groups or of reagents may be necessary and/or desirable. The introduction of conventional protective groups as well as their removal may be performed by methods well-known to those skilled in the art.

If the substituted azetidine compounds of general formula (I) themselves are obtained in form of a mixture of stereoisomers, particularly enantiomers or diastereomers, said mixtures may be separated by standard procedures known to those skilled in the art, e.g. chromatographic methods or fractunalized crystallization with chiral reagents. It is also possible to obtain pure stereoisomers via stereoselective synthesis.

Substituted azetidine compounds of general formula I, which comprise nitrogen-atom containing saturated, unsaturated or aromatic rings may also be obtained in the form of their N-oxides by methods well known to those skilled in the art.

In a further aspect the present invention also provides a process for the preparation of salts of substituted azetidine compounds of general formula (I), stereoisomers thereof or N-oxides thereof, wherein at least one compound of general formula (I) having at least one basic group is reacted with at least one inorganic and/or organic acid, preferably in the presence of a suitable reaction medium. Suitable reaction media include, for example, any of the ones given above. Suitable inorganic acids include hydrochloric acid, hydrobromic acid, phosphoric acid, sulfuric acid, nitric acid, suitable organic acids are e.g. citric acid, maleic acid, fumaric acid, tartaric acid, or derivatives thereof, p-toluenesulfonic acid, methanesulfonic acid or camphersulfonic acid.

In yet a further aspect the present invention also provides a process for the preparation of salts of substituted azetidine compounds of general formula (I), stereoisomers thereof or N-oxides thereof, wherein at least one compound of general formula (I) having at least one acidic group is reacted with one or more suitable bases, preferably in the presence of a suitable reaction medium. Suitable bases are e.g. hydroxides, carbonates or alkoxides, which include suitable cations, derived e.g. from alkaline metals, alkaline earth metals or organic cations, e.g. [NHₙR₄₋ₙ]⁺, wherein n is 0, 1, 2, 3 or 4 and R represents a branched or unbranched C₁₋₄-alkyl-radical. Suitable reaction media are, for example, any of the ones given above.

Solvates, preferably hydrates, of the substituted azetidine compounds of general formula (I), of corresponding stereoisomers, corresponding N-oxides thereof, or of corresponding salts thereof may-also be obtained by standard procedures known to those skilled in the art.

The purification and isolation of the inventive substituted azetidine compounds of general formula (I), of a corresponding stereoisomer, or salt, or solvate or any intermediate thereof may, if required, be carried out by conventional methods known to those skilled in the art, e.g. chromatographic methods or recrystallization.

The substituted azetidine compounds of general formula (I), their N-oxides, their stereoisomers, corresponding salts thereof and corresponding solvates are toxicologically acceptable and are therefore suitable as pharmaceutical active substances for the preparation of medicaments.

It has surprisingly been found that the substituted compounds of general formula I given above, stereoisomers thereof, corresponding N-oxides, corresponding salts and corresponding solvents have a high affinity to cannabinoid receptors, particularly cannabinoid 1 (CB₁)-receptors, i.e. they act as antagonists on these receptors.

Thus, an other aspect of the present invention is a medicament comprising at least one substituted azetidine compound of general formula I given above including the afore disclaimed compounds, optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, a corresponding salt thereof, or a corresponding solvate thereof, and optionally one or more pharmaceutically acceptable excipients.

Said medicament may comprise any combination of one or more of the substituted azetidine compounds of general formula I given above, stereoisomers thereof, corresponding N-oxides thereof, physiologically acceptable salts thereof or physiologically acceptable solvates thereof.

Preferably said medicament is suitable for the modulation (regulation) of cannabinoid-receptors, preferably cannabinoid 1 (CB₁) receptors, for the prophylaxis and/or treatment of disorders of the central nervous system, disorders of the immune system, disorders of the cardiovascular system, disorders of the endocrinous system, disorders of the respiratory system, disorders of the gastrointestinal tract or reproductive disorders.

More preferably said medicament is suitable for the prophylaxis and/or treatment of one or more disorders selected from the group consisting of psychosis, schizophrenia, anxiety, depression, epilepsy, neurodegenerative disorders, cerebellar disorders, spinocerebellar disorders, cognitive disorders, cranial trauma, panic attacks, peripheric neuropathy, inflammation, glaucoma, migraine, Morbus Parkinson, Morbus Huntington, Morbus Alzheimer, Raynaud's disease, tremblement disorders, compulsive disorders, senile dementia, thymic disorders, tardive dyskinesia, bipolar disorders, cancer, medicament-induced movement disorders, dystonia, endotoxemic shock, hemorragic shock, hypotension, insomnia, immunologic disorders, sclerotic plaques, vomiting, diarrhea, asthma, food intake disorders; preferably bulimia, anorexia, cachexia, obesity, type II diabetus mellitus (non-insuline dependent diabetes mellitus), memory disorders, pruritus, alcoholism, drug addiction, medicament addiction, preferably abuse of one or more of medicaments selected from the group consisting of opioids, barbiturates, cannabis, cocaine, amphetamines, phencyclidine, hallucinogens and benzodiazepines, pain, or for potentiation of the analgesic effect of narcotic and non-narcotic analgesics, or for influencing intestinal transit.

Particularly preferably the inventive medicament is suitable for the prophylaxis and/or treatment of pain, of food intake disorders, preferably bulimia, anorexia, cachexia, obesity or type II diabetus mellitus (non-insuline dependent diabetes mellitus), preferably diabetes, psychosis, alcoholism, drug addiction and/or medicament addiction, preferably drug addiction, diarrhea and/or pruritus.

Most preferably the inventive medicament is suitable for the prophylaxis and/or treatment of one or more disorders selected from the group consisting of obesity, psychosis and/or drug addiction.

Another aspect of the present invention is the use of at least one substituted azetidine compound of general formula I including the afore disclaimed compounds, optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, a corresponding salt thereof, or a corresponding solvate thereof, and optionally one or more pharmaceutically acceptable excipients, for the preparation of a medicament for the modulation of cannabinoid-receptors, preferably cannabinoid 1 (CB₁) receptors, for the prophylaxis and/or treatment of disorders of the central nervous system, disorders of the immune system, disorders of the cardiovascular system, disorders of the endocrinous system, disorders of the respiratory system, disorders of the gastrointestinal tract or reproductive disorders.

The use of at least one substituted azetidine compound of general formula I including the afore disclaimed compounds, optionally in form of one of the stereoisomers, preferably enantiomers or diastereomers, a racemate or in form of a mixture of at least two of the stereoisomers, preferably enantiomers and/or diastereomers, in any mixing ratio, or a corresponding N-oxide thereof, a corresponding salt thereof, or a corresponding solvate thereof, and optionally one or more pharmaceutically acceptable excipients, for the preparation of a medicament for the prophylaxis and/or treatment of one or more disorders selected from the group consisting of psychosis, schizophrenia, anxiety, depression, epilepsy, neurodegenerative disorders, cerebellar disorders, spinocerebellar disorders, cognitive disorders, cranial trauma, panic attacks, peripheric neuropathy, glaucoma, migraine, Morbus Parkinson, Morbus Huntington, Morbus Alzheimer, Raynaud's disease, tremblement disorders, compulsive disorders, senile dementia, thymic disorders, tardive dyskinesia, bipolar disorders, cancer, medicament-induced movement disorders, dystonia, endotoxemic shock, hemorragic shock, hypotension, insomnia, immunologic disorders, sclerotic plaques, vomiting, diarrhea, asthma, food intake disorders, preferably for the regulation of appetite, for the maintenance, increase or reduction of body weight, bulimia, anorexia, cachexia, obesity, type II diabetus mellitus (non-insuline dependent diabetes mellitus), memory disorders, alcoholism, pruritus, drug addiction, medicament addiction, preferably abuse of one or more of medicaments selected from the group consisting of opioids, barbiturates, cannabis, cocaine, amphetamines, phencyclidine, hallucinogens and benzodiazepines, paink, or for potentiation of the analgesic effect of narcotic and non-narcotic analgesics, or for influencing intestinal transit is particularly preferred.

The medicament according to the present invention may be in any form suitable for the application to humans and/or animals, preferably humans including infants, children and adults and can be produced by standard procedures known to those skilled in the art. The composition of the medicament may vary depending on the route of administration.

The medicament of the present invention may for example be administered parentally in combination with conventional injectable liquid carriers, such as water or suitable alcohols. Conventional pharmaceutical excipients for injection, such as stabilizing agents, solubilizing agents, and buffers, may be included in such injectable compositions. These medicaments may for example be injected intramuscularly, intraperitoneally, or intravenously.

Medicaments according to the present invention may also be formulated into orally administrable compositions containing one or more physiologically compatible carriers or excipients, in solid or liquid form. These compositions may contain conventional ingredients such as binding agents, fillers, lubricants, and acceptable wetting agents. The compositions may take any convenient form, such as tablets, pellets, capsules, lozenges, aqueous or oily solutions, suspensions, emulsions, or dry powdered forms suitable for reconstitution with water or other suitable liquid medium before use, for immediate or retarded release.

The liquid oral forms for administration may also contain certain additives such as sweeteners, flavoring, preservatives, and emulsifying agents. Non-aqueous liquid compositions for oral administration may also be formulated, containing edible oils. Such liquid compositions may be conveniently encapsulated in e.g., gelatin capsules in a unit dosage amount.

The compositions of the present invention may also be administered topically or via a suppository.

The daily dosage for humans and animals may vary depending on factors that have their basis in the respective species or other factors, such as age, sex, weight or degree of illness and so forth. The daily dosage for humans may preferably be in the range from 1 to 2000, preferably 1 to 1500, more preferably 1 to 1000 milligrams of active substance to be administered during one or several intakes per day.

Pharmacological Methods

I. In-vitro Determination of Affinity to CB1/CB2-Rezeptors

The in-vitro determination of the affinity of the inventive substituted azetidine compounds to CB₁/CB₂-Receptors is carried out as described in the publication of Ruth A. Ross, Heather C. Brockie et al., “Agonist-inverse agonist characterization at CB₁ and CB₂ cannabinoid receptors of L-759633, L759656 and AM630”, British Journal of Pharmacology, 126, 665-672, (1999). The respective parts of the descriptions are hereby incorporated by reference and form part of the present disclosure. The transfected human CB₁ and CB₂ receptor are obtained from Receptor Biology, Inc. The radioligand used for both receptors is [³H]-CP55940.

II. In-vivo Bioassay System for Determination of Cannabinoid Activity

Mouse Tetrad Model

Substances with affinity for cannabinoid receptors are known to produce a wide range of pharmacological effects. It is also known that intravenous administration of a substance with affinity for cannabinoid receptors in mice produces analgesia, hypothermia, sedation and catalepsy. Individually, none of these effects can be considered as proof that a tested substance has affinity for cannabinoid-receptors, since all of these effects are common for various classes of centrally active agents. However, substances, which show all of these effects, i.e. substances that are active in this so-called tetrad model are considered to have affinity for the cannabinoid receptors. It has further been shown that cannabinoid receptor antagonists are higly effective in blocking the effects of a cannabinoid agonist in the mouse tetrad model.

The tetrad model is described, for example, in the publication of A. C. Howlett et al, International Union of Pharmacology XXVII. Classification of Cannabinoid Receptors, Pharmacol Rev 54, 161-202, 2002 and David R. Compton et al., “In-vivo Characterization of a Specific Cannabinoid Receptor Antagonist (SR141716A): Inhibition of Tetrahydrocannbinol-induced Responses and Apparent Agonist Activity”, J. Pharmacol. Exp. Ther. 277 , 2, 586-594, 1996. The corresponding parts of the description are hereby incorporated by reference.

Material and Methods

Male NMRI mice with a weight of 20-30 g (Harlan, Barcelona, Spain) are used in all of the following experiments.

Before testing in the behavioral procedures given below, mice are acclimatized to the experimental setting. Pre-Treatment control values are determined for analgesia hot plate latency (in seconds), rectal temperature, sedation and catalepsy.

In order to determine the agonistic activty of the substance to be tested, the mice are injected intravenously with the substance to be tested or the vehicle alone. 15 minutes after injection, latency in hot plate analgesia is measured. Rectal temperature, sedation and catalepsy are measured 20 minutes after injection.

In order to determine the antagonistic activity the identical procedure is used as for the determination of the agonistic effects, but with the difference that the substance to be evaluated for its antagonistic activity is injectected 5 minutes before the intravenous injection of 1.25 mg/kg Win-55,212 a known cannabinoid-receptor agonist.

Hot Plate Analgesia

The hot plate analgesia is determined according to the method described in Woolfe D. et al. “The evaluation of analgesic action of pethidine hydrochloride (Demerol)”, J. Pharmacol. Exp. Ther. 80, 300-307, 1944. The respective description is hereby incorporated by reference and forms part of the present disclosure.

The mice are placed on a hot plate (Harvard Analgesimeter) at 55±0.5° C. until they show a painful sensation by licking their paws or jumping and the time for these sensations to occur is recorded. This reading is considered the basal value (B). The maximum time limit the mice are allowed to remain on the hot plate in absence of any painful response is 40 seconds in order to prevent skin damage. This period is called the cut-off time (PC).

Fifteen minuts after the administration of the substance to be tested, the mice are again placed on the hot plate and the afore described procedure is repeated. This period is called the post-treatment reading (PT).

The degree of analgesia is calculated from the formula:
% MPE of Analgesia=(PT-B)/(PC-B)×100
MPE=Maximum possible effect.

Determination of Sedation and Ataxia

Sedation and ataxia is determined according to the method described in Desmet L. K. C. et al. “Anticonvulsive properties of Cinarizine and Flunarizine in Rats and Mice”, Arzneim.-Forsch. (Frug Res) 25, 9, 1975. The respective description is hereby incorporated by reference and forms part of the present disclosure.

The chosen scoring system is

0: no ataxia;

1: doubful;

2: obvious calmness and quiet;

3 pronounced ataxia;

prior to as well as after treatment.

The percentage of sedation is determined according to the formula:
% of sedation=arithmetic mean/3×100

Hypothermia:

Hypothermia is determined according to the method described in David R. Compton et al. “In-vivo Characterization of a Specific Cannabinoid Receptor Antagonist (SR141716A) Inhibition of Tetrahydrocannbinol-induced Responses and Apparent Agonist Activity”, J. Pharmacol Exp Ther. 277, 2, 586-594, 1996. The respective description is hereby incorporated by reference and forms part of the present disclosure.

The base-line rectal temperatures are determined with a thermometer (Yello Springs Instruments Co., Panlabs) and a thermistor probe inserted to 25 mm before the administration of the substance to be tested. Rectal temperature is again measured 20 minutes after the administration of the substances to be tested. The temperature difference is calculated for each animal, whereby differences of ≧−2° C. are considered to represent activity.

Catalepsy:

Catalepsy is determined according to the method described in Alpermann H. G. et al. “Pharmacological effets of Hoe 249: A new potential antidepressant”, Drugs Dev. Res. 25, 267-282. 1992. The respective description is hereby incorporated by reference and forms part of the present disclosure.

The cataleptic effect of the substance to be tested is evaluated according to the duration of catalepsy, whereby the animals are placed head downwards with their kinlegs upon the top of the wooden block.

The chosen scoring system is:

Catalepsy for:

more than 60 seconds=6; 50-60 seconds=5, 40-50 seconds=4, 30-40 seconds=3, 20-30 seconds=2, 5-10 seconds=1, and less than 5 seconds=0.

The percentage of catalepsy is determined according ot the following formula:
% Catalepsy=arithmetic mean/6×100

The present invention is illustrated below with the aid of examples. These illustrations are given solely by way of example and do not limit the general spirit of the present invention.

### EXAMPLES

The following examples (a)-(i) show the preparation of selected intermediate compounds used in the synthesis of the inventive azetidine compounds.

### (a).—threo-3-Bromo-1,2-epoxybutane

Br₂ was added dropwise to a solution of 20.4 g (0.284 mol) trans-2-buten-1-ol in 60 ml of chloroform until the solution assumed a slight coloration (theoretical amount of Br₂: 45.4 g, 0.284 mol). Crotyl alcohol was then added dropwise until the solution turned transparent again. This reaction mixture was maintained at room temperature (approximately 25° C.) for 15 minutes, the solvent evaporated off and a dark liquid residue was obtained. This crude 2,3-dibromo-1-butanol was dissolved in 140 ml of diethyl ether and 16 g (0.284 mol) of potassium hydroxide in 170 ml of water were added to the resulting solution. The mixture was stirred for 2 hours at room temperature, the two layers separated and the organic layer washed with water. The solvent was evaporated off and the residue distilled under vacuum to yield 24 g (56% of theory) of threo-3-bromo-1,2-epoxybutane with a boiling point of 55-60° C. at 25 mm Hg.

¹H NMR (CDCl₃, δ): 1.68 (d, 3H ,J=7 Hz); 2.69 (dd, 1H, J=5 y 2.5 Hz); 2.88 (dd, 1H,J=5 y 4 Hz); 3.18 (ddd.1H, J=7, 4 y 2.5 Hz ); 3.86 (q. 1H, J=7 Hz)

### (b).—trans-1-Diphenylmethyl-3-hydroxy-2-methylazetidine

A solution of threo-3-bromo-1,2-epoxybutane (9.8 g, 64.90 mmol) and aminodiphenylmethane (11.8 g, 64.5 mmol) in 70 ml of methanol was kept stirring for 80 hours at room temperature and 72 hours under reflux. The reaction mixture was then evaporated to dryness and the viscous residue treated with diethyl ether and water. The aqueous layer was alkalinized with potassium carbonate and extracted with diethyl ether to yield 9.4 g (61% of theory) of trans-1-diphenylmethyl-3-hydroxy-2-methylazetidine. The corresponding hydrochloride salt was obtained by dissolving a solution of the compound in an ethanol solution saturated with HCl gas and subsequent elimination of the solvent in vacuum.

Melting point of hydrochloride: 100-103° C.,

IR (film, cm⁻¹): 3400, 1450, 1156, 749. 702,

¹H NMR (CDCl₃, δ): 0.75 (d, J=6 Hz); 2.40 (b, 1H); 2.56 (t, 1H, J=6 Hz); 3.02 (q. 1H,J=6 Hz); 3.64 (t, 1H, J=6 Hz); 3.87 (quint., 1H, J=6 Hz); 4.34 (s, 1H); 7.27 (m. 10 H).

### (c) trans-1-Diphenylmethyl-2-methyl-3-methylsulphonyloxyazetidine

50 g (0.495 mol) of triethylamine were added to a solution of 77.33 g (0.329 mol) trans-1-diphenylmethyl-3-hydroxy-2-methylazetidine in 600 ml of dichloromethane and the resulting mixture was cooled to 0° C. The temperature was maintained, a solution of 50 g (0.437 mol) of mesyl chloride added dropwise and the resulting mixture was left stirring for 24 hours at room temperature. The resulting solution was washed twice with water (300 ml), dried with anhydrous sodium sulphate and evaporated to yield an oil, which, when crystallized with petroleum ether, gave 104.6 g (96% of theory) of trans-1-diphenylmethyl-2-methyl-3-methylsulphonyloxyazetidine with a melting point of 68-71° C.

IR (film, cm⁻¹): 1361, 1339, 1178, 1152, 708,

¹H NMR (CDCl₃, δ): 0.63 (d, 3H, J=7 Hz); 2.85 (t. 1H, J=6 Hz); 2.96 (s, 3H); 3.62 (t, 2H, J=6 Hz), 4.39 (s. 1H); 4.55 (quint., 1H, J=6 Hz); 7.23 (m, 10H).

### (d) trans-3-Amino-1-diphenylmethyl-2-methylazetidine

31 g (93.65 mmol) of trans-1-diphenylmethyl-2-methyl-3-methylsulphonyloxyazetidine were dissolved in a mixture of 150 ml isopropanol and 100 ml of 30% (weight/weight) aqueous ammonia solution. The resulting solution was heated to 70° C. for 2-3 hours while the reaction was monitored by thin-layer chromatography. After completion of the reaction, the reaction mixture was evaporated until the isopropanol was removed completely (approximately ⅓ of the volume) and the residue was extracted with diethyl ether and water. The aqueous layer was alkalinized and extracted with dichloromethane to yield 10 g of the desired compound. The etheric layer of the first extraction was acidified with dilute 5% (volume/volume) acetic acid, the acidic layer alkalinized with sodium hydroxide and extracted wilh dichloromethane, to yield 6.3 g of the compound giving a total of 16.3 g (70% of theory) of trans-3-amino-1-diphenylmethyl-2-methylazetidine having a melting point of 68-69° C.

M.p. of corresponding dihydrochloride: 150-153° C.,

IR ( film, cm⁻¹): 3270, 1450. 702,

¹H NMR (CDCl₃, δ): 0.64 (d, 3H, J=7 Hz), 2.20 (q, 1H, J=7 Hz); 2.63 (t, 1H, J=7 Hz); 2.90 (quinl., 1 H, J=7 Hz), 3.50 (t, 1H, J=7 Hz), 4.20 (s, 1H); 7.20 (m, 10H).

### (e).—(2S,3R)-1-Diphenylmethyl-2-methyl-3-methylsulphonyloxyazetidine

The enantiomer (2S,3R)-1-diphenylmethyl-2-methyl-3-methylsulphonyloxyazetidine was obtained by the same procedure as described above for the preparation of the trans-racemate but using (2S,3R)-1-diphenylmethyl-3-hydroxy-2-methylazetidine. The compound (2S,3R)-1-diphenylmethyl-3-hydroxy-2-methylazetidine was obtained via the optical resolution of trans-1-diphenylmethyl-3-hydroxy-2-methylazetidine with (+)-(1S)-camphersulfonic acid, as described in the literature publication of J. Frigola et al., J. Med. Chem., 1995, 38, 1203-1215.

### (f).—(2S,3R)-3-amino-1-diphenylmethyl-2-methylazetidine

The compound (2S,3R)-3-amino-1-diphenylmethyl-2-methylazetidine was obtained as described above under (d) using (2S,3R)-1-diphenylmethyl-2-methyl-3-methylsulphonyloxyazetidine.

### (g).—(2S,3R)-N-(1-Benzhydryl-2-methyl-azetidin-3-yl)-2,2,2-trifluoro-acetamide

A solution of trifluoracetic anhydride (18.3 ml, 131.7 mmoles) in 25 ml of dichloromethane is added dropwise under stirring and cooling to approximately 0° C. to a solution of (2S,3R)-3-amino-1-diphenylmethyl-2-methylazetidine (16.6 g, 65.9 mmoles) in dichloromethane (90 ml).

After the addition has been completed, the reaction mixture was stirred at room temperature for two hours, ice-cooled water was added, and the different phases were separated. The organic phase was washed with a solution of sodium bicarbonate 10% (weight/weight), followed by a saturated solution of sodium chloride, dried and evaporated to dryness under reduced pressure to give 23.35 g (yield 92% of theory) of (2S,3R)-N-(1-Benzhydryl-2-methyl-azetidin-3-yl)-2,2,2-trifluoro-acetamide in form of an oil.

IR (film, cm⁻¹): 3260, 1710, 1660, 1230.

Said product was dissolved in dried ethanol and a solution of hydrochloride gas in diethylether was added, and the resulting solution was evaporated to dryness. The corresponding hydrochloride was obtained in form of a white solid with a melting point of 208-212° C.

IR of hydrochloride (KBr, cm⁻¹): 3319, 1700, 1562, 1213, 1187, 700.

### (h).—(2S,3R)-2,2,2-Trifluoro-N-(2-methyl-azetidin-3-yl)-acetamide, hydrochloride

(21.9 g, 57 mmoles) of (2S,3R)-N-(1-Benzhydryl-2-methyl-azetidin-3-yl)-2,2,2-trifluoro-acetamide hydrochloride was dissolved in 300 ml methanol, Pd(OH)₂/C (20%, 4.4 g, humidity 50%) were added, and the resulting mixture was treated with H₂ at room temperature under a pressure of 150 psi for 15 minutes. The reaction mixture was filtered, the solvent evaporated off, and the residue was washed with toluene to give 12.3 g (yield 99%) of (2S,3R)-2,2,2-Trifluoro-N-(2-methyl-azetidin-3-yl)-acetamide with a melting point of 219-221° C.

IR (KBr, cm⁻¹): 3244, 2895, 1727, 1563, 1213, 1177

### (i).—Bis-(4-clorophenyl)methyl bromide

A solution of 4,4′-dichlorobenzhydrol (5 g, 19.8 mmoles) and acetyl bromide (6 ml, 80 mmoles) in benzene (40 ml) is heated to reflux for three hours. The reaction mixture is evaporated to dryness, and the resulting solid (6.2 g, yield 100%) is used directly for further synthesis without purification.

¹H NMR (CDCl₃, δ): 6.2 (s, 1H), 7.3 (d, J=8.7 Hz, 4H), 7.36 (d, J=8.7 Hz, 4H).

### Example 1

### 4-Fluoro-benzenesulfonic acid-trans-1-[bis-(4-chloro-phenyl)-methyl]-2-methyl-azetidin-3-yl ester (1a) 2,2-dimethyl-propionic acid-trans-1-benzhydryl-2-methyl-azetidin-3-yl-ester

To a solution of the hydrochloride salt of trans-1-benzhydryl-2-methyl-azetidin-3-ol (3.73 g, 12.8 mmoles) prepared according to example (b) given above in 80 ml pyridine, 4.4 ml of triethylamine were added and the mixture was cooled to approximately 0° C. Afterwards, a solution of (2.4 ml, 19.2 mmoles) trimethylacetylchloride (pivaloylchloride) was added, and the reaction mixture was heated to approximately 70° C. under an inert gas atmosphere for eight hours. The solvent was evaporated off, and the residue was dissolved in diethylether, washed with water, and the ether evaporated to yield the free base.

¹H NMR (CDCl₃, δ): 0.8 (d, J=6.3 Hz, 3H), 1.2 (s, 9H), 2.6 (m, 1H), 3.15 (m, 1H), 3.7 (dd, J=7.0 y 7.5 Hz, 1H), 4.3 (s, 1H); 4.6 (dd, J=6.0, 6.6 y 12.6 Hz), 7.25 (m, 6H), 7.4 (m, 4H).

The crude material is dissolved in 15 ml of ethanol, and an ethanolic solution saturated with hydrochloride gas is added to precipitate the corresponding hydrochloride salt, which is filtered off and washed with diethylether to yield 3.95 g (yield 85% of theory) of the hydrochloride salt having a melting point of 163-166° C.

¹H NMR (d₆-DMSO, δ): 1.15 (s+d, 12H), 3.8 (m, 1H), 4.2 (m,1H), 4.6 (m,1H), 4.9 (m,1H), 5.8 (d, J=10 Hz, 1H), 7.4 (m, 6H), 7.75 (m,4H), 12.2 (s, 1H).

### (1b) 2,2-dimethyl-propionic acid (trans-2-methyl-azetidin-3-yl)ester hydrochloride

2,2-dimethyl-propionic acid 2-methyl-azetidin-3-yl ester hydrochloride was obtained from the compound obtained according to step (1a) following treatment described in example (h) given above (yield 94% of theory).

¹H NMR (CDCl₃, δ): 1.2 (s, 9H), 1.7 (d, J=6.9 Hz, 3H), 3.9 (m, 1H), 4.2 (m, 1H), 4.4 (m, 1H), 4.9 (m, 1H), 9.8-10.1 (b, 2H).

### (1c) trans-1-[Bis-(4-chloro-phenyl)-methyl]-2-methyl-azetidin-3-yl-2,2-dimethyl-propionic acid

Said compound was obtained according to the method described in example 2 given below using bis-(4-chlorophenyl)methyl bromide which was prepared according to example (i) given above (yield 95% of theory).

¹H NMR (CDCl₃, δ): 0.8 (d, J=6.5 Hz, 3H), 1.2 (s, 9H), 2.6 (m, 1H), 3.15 (m, 1H), 3.7 (m, 1H), 4.3 (s, 1H), 4.6 (m, 1H), 7.2-7.35 (m, 8H)

### (1d) 1-[Bis-(4-chloro-phenyl)-methyl]-2-methyl-azetidin-3-ol

This compound was obtained via the hydrolysis of the ester prepared according to step (1c) given above, which was dissolved in ethanol comprising 10% by weight of sodium hydroxide. The reaction mixture was kept at room temperature (approximately 25° C.) overnight. 1-[Bis-(4-chloro-phenyl)-methyl]-2-methyl-azetidin-3-ol was obtained in a yield of 89%.

¹H NMR(CDCl₃, δ): 0.8 (d, J=6.2 Hz, 3H), 2.5 (m, 1H), 3.0 (m, 1H), 3.6 (m, 1H), 3.9 (m, 1H), 4.3 (s, 1H), 7.2-7.3 (m, 8H)

### (1e) trans-4-Fluoro-benzenesulfonic acid 1-[bis-(4-chloro-phenyl)-methyl]-2-methyl-azetidin-3-yl ester

Said compound was obtained according to the method described in example 5 given below.

Melting poing=105-107° C.,

IR (KBr, cm⁻¹): 1494, 1370, 1187, 1159, 1089, 1015,

¹H RMN (CDCl₃, δ): 0.7 (d, J=6.2 Hz, 3H), 2.7 (m, 1H), 3.3 (m, 1H), 3.5 (m, 1H), 4.3 (s, 1H), 4.4 (m, 1H), 7.2 (m, 10H), 7.9 (m, 2H).

### Example 2

### (2R,3S)-N-{1-[Bis-(4-chloro-phenyl)-methyl]-2-methyl-azetidin-3-yl}-2,2,2-trifluoro-acetamide

A mixture of bis-(4-chlorophenyl)methyl bromide (4 g, 12.6 mmoles), the hydrochloride of (2R,3S)-2-methyl-3-trifluoroacetylamino azetidine (2.3 g, 10.5 mmoles) and potassium carbonate (16.5 g, 120 mmoles) in 400 ml of acetonitrile was heated to reflux for 24 hours. The reaction mixture was filtered and the resulting clear solution evaporated to dryness. The crude material is cristallized from ethylacetate to yield 2.9 g of the product. Another 0.4 g of the product were obtained by working up the mother liquors. The overall yield of the comound (2R,3S)-N-{1-[bis-(4-chloro-phenyl)-methyl]-2-methyl-azetidin-3-yl}-2,2,2-trifluoro-acetamide was 3.3 g (yield 67% of theory).

IR (KBr, cm⁻¹): 3262, 3080, 1678, 1495, 1200, 1080, 799,

¹H RMN (d₆-DMSO+TFA, δ): 1.0 (d, J=5.6 Hz, 3H). 3.95, dd, J=6.8 y 17.9 Hz, 1H), 4.3 (2dd, 2H), 4.6 (dd, J=6.8 y 13.2 Hz, 1H), 5.7 (s, 1H), 7.4-7.5 (m, 8H), 9.8 (d, J=5.3 Hz, 1H).

### Example 3

### (2R,3S)-1-[Bis-(4-chlorophenyl)-methyl]-2-methyl-azetidin-3-yl amine

(2R,3S)-N-{1-[bis-(4-chloro-phenyl)-methyl]-2-methyl-azetidin-3-yl}-2,2,2-trifluoro-acetamide (2.8 g, 6.7 mmoles) was suspended in a solution of sodium hydroxide (25 ml, ≈60 mmoles) and 60 ml ethanol and stirred at room temperature (approximately 25° C.), whereby the gradual dissolution was observed until a transparent solution was obtained. After three hours, the solvent was evaporated under reduced pressure and the resulting basic solution was extracted with ethylacetate, washed with water, dried over sodium sulfate and evaporated to dryness. (2R,3S)-1-[Bis-(4-chlorophenyl)methyl]-2-methyl-azetidin-3-yl amine was obtained in form of an oil (1.95 g, yield 91% of theory).

IR (KBr, cm⁻¹): 3369, 1495, 1110, 1030, 790,

¹H RMN (CDCl₃, δ): 0.75 (d, J=5.8 Hz, 3H), 2.3 (m, 1H), 2.7 (m, 1H), 3.0 (m, 1H), 3.6 (m, 1H), 4.2, (s, 1H ), 9, 7.2-7.3 (m, 8H).

The product was dissolved in an ethanolic solution saturated with hydrochloride gas and afterwards, the solvent was evaporated to yield the corresponding dihydrochloride salt.

IR (KBr, cm⁻¹): 3380, 1488, 1094, 1014

[α]_{D} −35.5 (c 1.0, MeOH)

### Example 5

### (2S,3R)-N-{1-[Bis 4-chlorophenyl)-methyl]-2-methylazetidin-3-yl}-4-fluoro-benzene sulfonamide

(2R,3S)-1-[bis-(4-chlorophenyl)methyl]-2-methyl-azetidin-3-yl amine (0.15 g, 0.47 mmoles) and triethylamine (84 μl, 0.59 mmoles) was dissolved in anhydrous tetrahydrofuran (6 ml). The mixture was cooled with an ice-bath to 0° C. and a solution of 4-benzene-sulphonylchloride (0.11 g, 0.54 mmoles) in THF (4 ml) was added dropwise. The cooling bath was removed, and the reaction mixture was allowed to warm up to room temperature (approximately 25° C.) and stirred overnight. The solvent was removed under reduced pressure, and the residue was re-dissolved in ethylacetate and water. The organic solution was washed with a saturated solution of sodium bicarbonate, water and dried over sodium sulfate and evaporated to dryness. 0.16 g of (2R,3S)-1-N-{1-[bis-(4-chlorophenyl)methyl]-2-methylazetidin-3-yl}-4-fluorobenzenesulfonamide were obtained (yield 75% of theory) with a melting point of 154-157° C.

IR (KBr, cm⁻¹): 3255, 1592, 1492, 1155, 1090, 802.

¹H RMN (CDCl₃, δ): 0.6 (d, J=5.8 Hz, 3H), 2.3 (m, 1H), 2.9 (m, 1H), 3.4 (m, 2H), 4.2 (s, 1H), 4.8 (d, J=8.4 Hz, 1H), 7.1-7.25 (m, 10H), 7.8 (m, 2H).

[α]_{D} −61.5 (c 1.0, MeOH).

The compounds prepared according to examples 1, 2, 3 and 5 given above are given in the following table 1. Also included are example compounds 4 and 6-31, which were prepared by analogous methods.

TABLE 1

| | | m.p. | IR | ¹H NMR |
|---|---|---|---|---|
| Ex. | | ° C. | (KBr, cm⁻¹) | (CDCl₃, δ) |
| 1 | 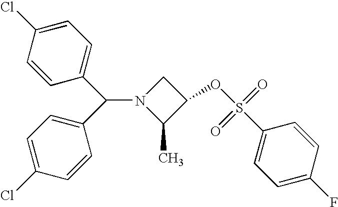 | 105-107 | 1494, 1370, 1187, 1159, 1089, 1015 | 0.7 (d, J=6.2 Hz, 3H), 2.7 (m, 1H), 3.3 (m, 1H), 3.5 (m, 1H), 4.3 (s, 1H), 4.4 (m, 1H), 7.2 (m, 10H), 7.9 (m, 2H) |
| 2 | 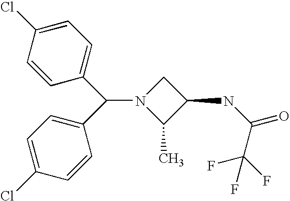 | 122-127 | 3262, 3080, 1678, 1495, 1200, 1080, 799 | (d₆-DMSO + TFA, δ): 1.0 (d, J=5.6 Hz, 3H), 3.95, dd, J=6.8 y 17.9 Hz, 1H), 4.3 (2dd, 2H), 4.6 (dd, J=6.8 y 13.2 Hz, 1H), 5.7 (s, 1H), 7.4-7.5 (m, 8H), 9.8 (d, J=5.3 Hz, 1H) |
| 3 | 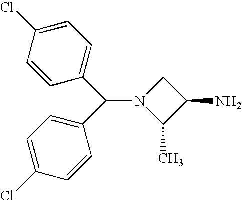 | 134-136 | 3369, 1495, 1110, 1030, 790 | 0.75 (d, J=5.8 Hz, 3H), 2.3 (m, 1H), 2.7 (m, 1H), 3.0 (m, 1H), 3.6 (m, 1H).4.2, (s, 1H), 9, 7.2-7.3 (m, 8H). |
| 4 | 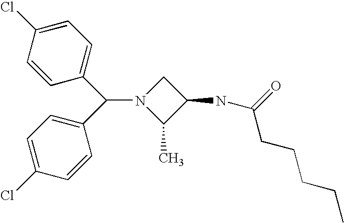 | amorph | 3385, 2930, 1686, 1493, 1092, 1013, 801 | 0.85 (m, 6H), 1.3 (m, 4H), 1.6 (m, 2H), 2.1 (t, J=7.5 Hz, 2H), 2.4 (t, J=7.5 Hz, 1H), 2.9 (m, 1H), 3.6 (t, J=7.5 Hz, 1H), 4.1 (m, 1H), 4.3 (s, 1H), 5.4 (d, J=6.8 Hz, 1H), 7.2 (m, 8H) |
| 5 | 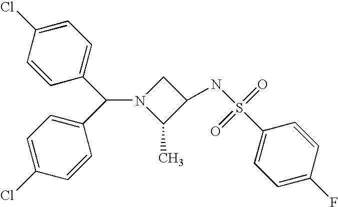 | 154-157 | 3255, 1592.1492, 1155, 1090, 802 | 0.6 (d, J=5.8 Hz, 3H), 2.3 (m, 1H), 2.9 (m, 1H), 3.4 (m, 2H), 4.2 (s, 1H), 4.8 (d, J=8.4 Hz, 1H), 7.1-7.25 (m, 10H), 7.8 (m, 2H) |
| 6 | 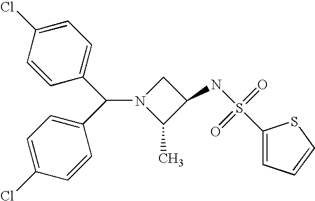 | 72-76 | 3262, 1489, 1155, 1089, 1014, 804 | 0.65 (d, J=6.2 Hz, H), 2.4 (t, J=7.4 Hz, 1H), 3.0 (m, 1H), 3.4-3.55 (m, 2H), 4.2 (s, 1H), 4.9 (d, J=8.8 Hz, 1H), 7 (t, J=4.0, 4.7 Hz, 1H), 7.2 (m, 8H), 7.6 (m, 2H) |
| 7 | 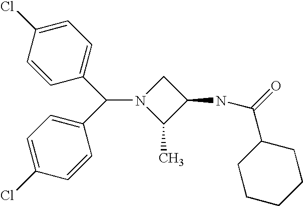 | 65-68 | 3293, 2929, 1638, 1542, 1489, 1089, 1014 | 0.8 (d, J=6.2 Hz, 3H), 1.2 (m, 6H), 1.4 (m, 4H), 2.0 (t, 1H), 2.45 (t, J=7.5 Hz, 1H), 2.9 (m, 1H), 3.6 (t, J=7.1 Hz, 1H), 4.25 (s, 1H), 5.5 (d, J=7.9, 1H), 7.3 (m, 8H) |
| 8 | 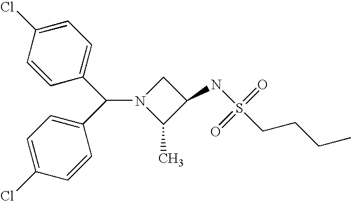 | (HCl) 90-96 | (HCl): 3422, 2961, 1496, 1330, 1143, 1093, 1014, 801 | (HCl, d₆DMSO): 0.8-1.6 (m, 10H), 3.0 (m, 2H), 3.6-3.9 (m, 2H), 4.1-4.5 (m, 2H), 5.8 (b, 1H), 7.5-8.0 (m, 9H), 12.2 (b, 1H) |
| 9 | 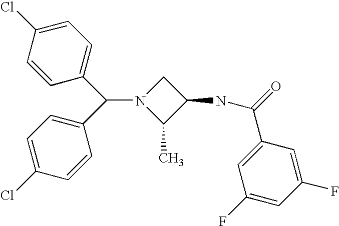 | 57-60 | 3293, 1638, 1596, 1544, 1489, 1332, 1089, 803 | 0.9 (d, J=5.8 Hz, 3H), 2.6 (m, 1H), 3.1 (m, 1H), 3.7 (m, 1H), 4.3 (m + s, 2H), 6.1 (b, 1H), 6.95 (m, 1H), 7.2 (m, 10H) |
| 10 | 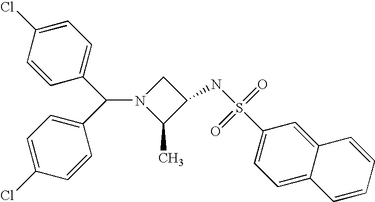 | 156-160 | 3231, 1488, 1330, 1159, 1086, 1013, 811 | 0.55 (d, J=6.2 Hz, 3H), 2.35 (t, J=7.6 Hz, 1H), 2.9 (m, 1H), 3.4-3.6 (m, 2H), 4.1 (s, 1H), 4.9 (d, J=9 Hz, 1H), 7.2 (m, 8H), 7.6 (m, 2H), 7.7 (m, 1H), 7.9 (m, 3H), 8.4 (s, 1H) |
| 11 | 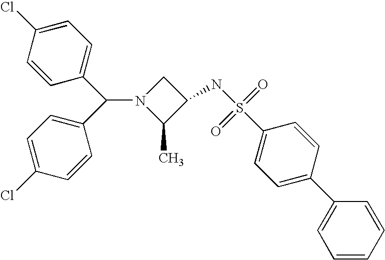 | 145-148 | 3220, 1590, 1488, 1321, 1161, 1089, 801 | 0.6 (d, J=6.2 Hz, 3H), 2.4 (t, J=7.3 Hz, 1H), 2.9 (m, 1H), 3.5 (m, 2H), 4.2 (s, 1H), 4.9 (d, J=8.8 Hz, 1H), 7.2 (m, 8H), 7.4 (m, 3H), 7.6 (m, 2H), 7.7 (m, 2H), 7.9 (m, 2H) |
| 12 | 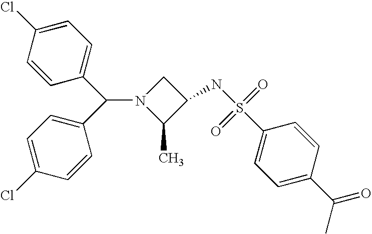 | 177-180 | 3224, 1694.1489, 1164.1088, 804 | 0.6 (d, J=6.1 Hz, 3H), 2.3 (t, J=7.3 Hz, 1H), 2.6 (s, 3H), 2.9 (m, 1H), 3.4 (m, 2H), 4.2 (s, 1H), 4.9 (d, J=9 Hz, 2H), 8 (d, J=7 Hz, 2H) |
| 13 | 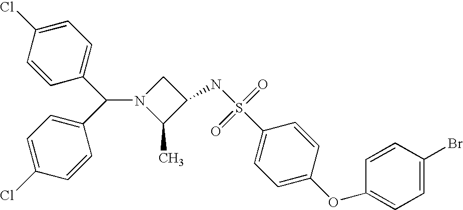 | 115-119 | 3220, 1578, 1481, 1246, 1157, 1090, 1012, 803 | 0.6 (d, J=6.2 Hz, 3H), 2.3 (m, 1H), 2.9 (m, 1H), 3.4 (m, 2H), 4.2 (s, 1H), 4.7 (d, J=8.6 Hz, 1H), 6.9 (d, J=9 Hz, 2H), 7 (d, J=9 Hz, 2H), 7.2 (m, 8H), 7.5 (d, J=9 Hz, 2H), 7.8 (d, J=9 Hz, 2H) |
| 14 | 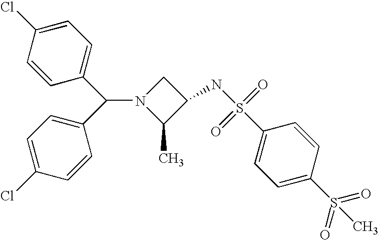 | 202-204 | 3239, 1490, 1321, 1167, 1091, 1014, 741 | 0.65 (d, J=6.1 Hz, 3H), 2.4 (m, 1H), 2.9 (m, 1H), 3.1 (s, 3H), 3.4 (m, 2H), 4.2 (s, 1H), 4.8 (d, J=8.5 Hz, 1H), 7.2 (m, 8H), 8 (2d, J=8.4 Hz, 4H) |
| 15 | 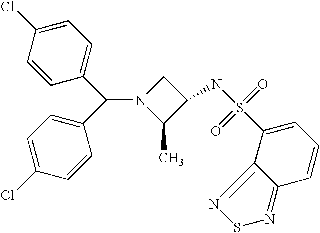 | 7616 | 3288, 1523, 1489, 1335, 1159, 1089, 804 | 0.45 (d, J=6 Hz, 3H), 2.3 (t, J=7.5 Hz, 1H), 2.9 (m, 1H), 3.3 (t, J=7.5 Hz, 1H), 3.5 (m, 1H), 4.1 (s, 1H), 5.6 (d, J=8.6 Hz, 1H), 7.2 (m, 8H), 7.7 (dd, J=7 y 9 Hz, 1H), 8.2 (m, 2H) |
| 16 | 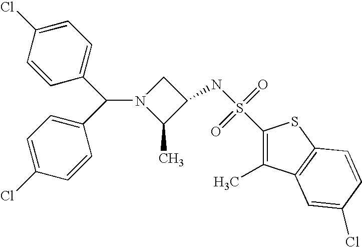 | 105-108 | 3233, 1488, 1338, 1157, 1087, 799 | 0.6 (d, J=5.9 Hz, 3H), 2.4 (t, J=7.5 Hz, 1H), 2.6 (s, 3H), 3 (m, 1H), 3.4-3.6 (m, 2H), 4.2 (s, 1H), 5.1 (d, J=9.3 Hz, 1H), 7.2 (m, 8H), 7.4 (d, J=8.5 Hz, 1H), 7.7 (m, 2H) |
| 17 | 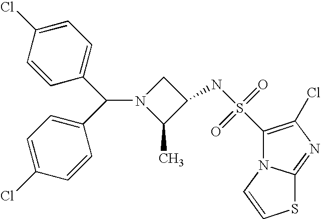 | 164-169 | 3448, 1489, 1459, 1340, 1250, 1178, 1142, 1089, | 0.6 (d, J=6.1 Hz, 3H), 2.5 (m, 1H), 3 (m, 1H), 3.5 (m, 2H), 4.2 (s, 1H), 5.2 (d, J=8.1 Hz, 1H), 7 (d, J=4.6 Hz, 1H), 7.2 (m, 8H), 7.9 (d, J=4.6 Hz, 1H) |
| 18 | 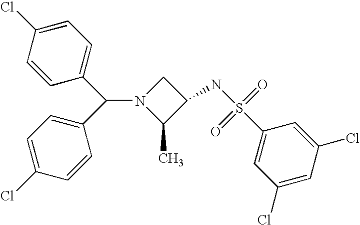 | 144-146 | 3293, 1569, 1489, 1334, 1166, 1140, 1089, 803 | 0.65 (d, J=6 Hz, 3H), 2.4 (m, 1H), 3 (m, 1H), 3.5 (m, 2H), 4.2 (s, 1H), 4.8 (d, J=8.5 Hz, 1H), 7.2 (m, 8H), 7.5 (s, 1H), 7.7 (s, 2H) |
| 19 | 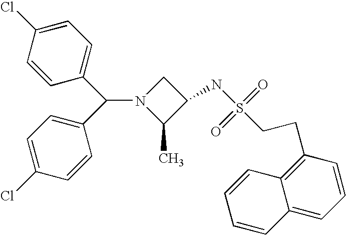 | 123-128 | 3263, 1489, 1320, 1150, 1088, 1014, 805 | 0.7 (d, J=6 Hz, 3H), 2 (t, J=7.5 Hz, 1H), 2.7 (m, 1H), 3.3-3.6 (m, 6H), 4 (s, 1H), 4.3 (d, J=9 Hz, 1H), 7.2 (m, 8H), 7.3-7.5 (m, 4H), 7.7 (d, J=7.7 Hz, 1H), 7.8 (d, J=7.2 Hz, 1H), 7.9 (d, J=7.5 Hz, 1H) |
| 20 | 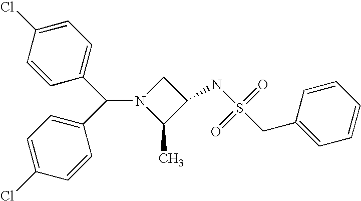 | 117-118 | 3319, 1489, 1328 1167, 1136, 1090, 804 | 0.7 (d, J=6 Hz, 3H), 2.4 (t, J=7.5 Hz, 1H), 2.9 (m, 1H), 3.35 (m, 1H), 3.5 (t, J=7.5 Hz, 1H), 4.2 (s, 1H), 4.3 (d, J=9.3 Hz, 1H), 7.2-7.4 (m, 13H) |
| 21 | 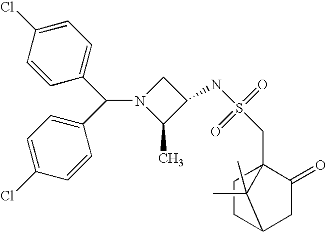 | 65-67 | 3279, 2961, 1743, 1489, 1331, 1147, 1089, 804 | 0.8 (m, 6H), 0.9 (d, J=7.2 Hz, 3H), 1.4 (m, 1H), 1.8-2.2 (m, 5H), 2.4 (m, 1H), 2.6 (m, 1H), 2.9-3.3 (m, 5.8 (m, 1H), 7.2 (m, 8H) |
| 22 | 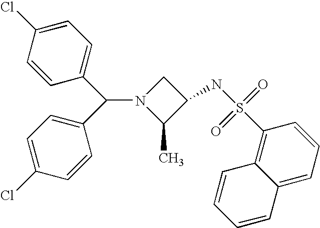 | 171-174 | 3292, 1489, 1328, 1162, 1135, 1089, 1015, 800 | 0.4 (d, J=6 Hz, 3H), 2.2 (t, J=7.5 Hz, 1H), 2.8 (m, 1H), 3.3 (t, J=7.5 Hz, 1H), 3.4 (m, 1H), 4.1 (s, 1H), 5 (d, J=9.4 Hz, 1H), 7.15 (m, 8H), 7.5-7.7 (m, 3H), 7.9 (d, J=8 Hz, 1H), 8 (d, J=7.7 Hz, 1H), 8.25 (d, J=7.3 Hz, 1H), 8.5 (d, J=8.1 Hz, 1H) |
| 23 | 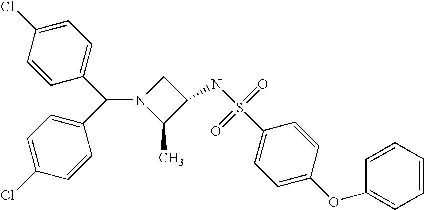 | 160-162 | 3225, 1583, 1488, 1323, 1244, 1159, 1091, 803 | 0.6 (d, J=6.3 Hz, 3H), 2.3 (m, 1H), 2.9 (m, 1H), 3.4 (m, 2H), 4.2 (s, 1H), 4.8 (d, J=8.7 Hz, 1H), 7 (m, 3H), 7.2 (m, 10H), 7.4 (m, 2H), 7.75 (d, J=8.8 Hz, 2H) |
| 24 | 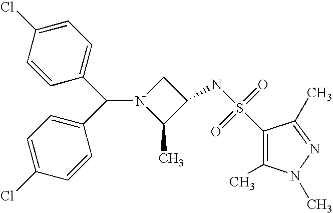 | 173-175 | 3290, 1490, 1323, 1152, 1088, 803 | 0.65 (d, J=6 Hz, 3H), 2.3 (s, 3H), 2.4 (m, 4H), 2.9 (m, 1H), 3.4 (m, 1H), 3.5 (t, J=7.1 Hz, 1H), 3.7 (s, 3H), 4.2 (s, 1H), 4.7 (d, J=9.3 Hz, 1H), 7.2 (m, 8H) |
| 25 | 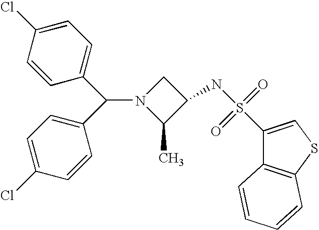 | 192-197 | 3295, 1489, 1334, 1159, 1088, 800, 753 | 0.5 (d, J=6 Hz, 3H), 2.3 (t, J=7.5 Hz, 1H), 2.9 (m, 1H), 3.4 (t, J=7.4 Hz, 1H), 3.5 (m, 1H), 4.1 (s, 1H), 5 (d, J=8.9 Hz, 1H), 7.2 (m, 8H), 7.5 (m,2H), 7.9 (d, J=7.1 Hz, 1H), 8.2 (d, J=8.6 Hz, 1H) |
| 26 | 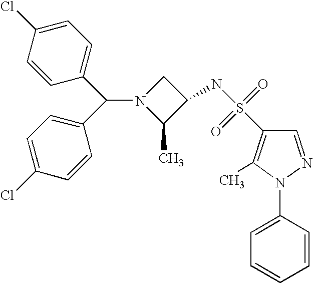 | 173-176 | 3233, 3155, 1501, 1488, 1327, 1156, 1089 | 0.7 (d, J=6 Hz, 3H), 2.4 (m + s, 4H), 3 (m, 1H), 3.5 (m, 2H), 4.2 (s, 1H), 4.9 (d, J=7.7 Hz, 1H), 7.2-7.4 (m, 10H), 7.5 (m, 3H), 7.8 (s, 1H) |
| 27 | 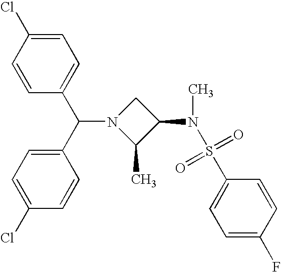 | 182-186 | 1591, 1491, 1347, 1166, 1086, 1012 | 0.7 (d, J=6.1 Hz, 3H), 2.6 (s, 3H), 2.8 (t, J=7 Hz, 1H), 3.2 (m, 1H), 3.4 (m, 2H), 4.3 (s, 1H), 7.1-7.3 (m, 10H), 7.7 (m, 2H) |
| 28 | 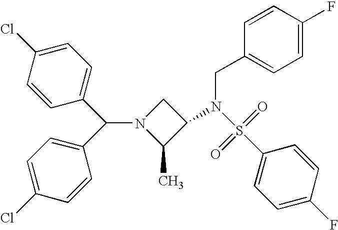 | 75-78 | 1592, 1509, 1493, 1349, 1220, 1156, 1089 | 0.6 (d, J=5.9 Hz, 3H), 2.6 (t, J=7.5 Hz, 1H), 3 (m, 1H), 3.2 (t, J=7.6 Hz, 1H), 3.9 (m, 1H), 4 (s, 1H), 4.4 (s, 2H), 7-7.3 (m, 14H), 7.8 (m, 2H) |
| 29 | 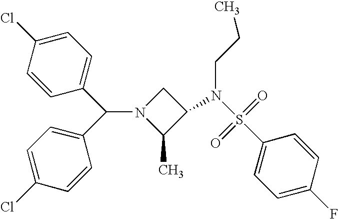 | 2967, 1592, 1492, 1351, 1157, 1088, 1014, 804 | 0.7 (d, J=6 Hz, 3H), 0.9 (t, J=7.3 Hz, 3H), 1.6 (m, 2H), 2.7 (t, J=7.7 Hz, 1H), 3 (m, 2H), 3.2 (m, 1H), 3.4 (t, J=7.2 Hz, 1H), 3.7 (m, 1H), 4.3 (s, 1H), 7.1-7.3 (m, 10H), 7.7 (m, 2H) | |
| 30 | 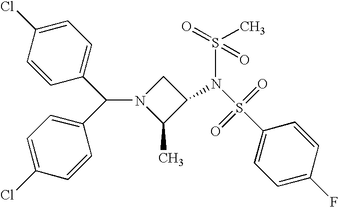 | amorph | 1592, 1493, 1370, 1169, 1157, 1088 | 0.6 (d, J=6.3 Hz, 3H), 3.3 (t, J=7.5 Hz, 1H), 3.35 (s, 3H), 3.45 (t, J=8.1 Hz, 1H), 3.9 (m, 1H), 4.4 (m, 1H), 4.5 (s, 1H), 7.2 (m, 10H), 8 (m, 2H) |
| 31 | 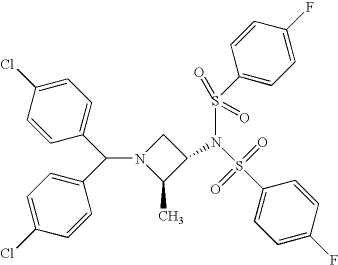 | 60-64 | 1591, 1493, 1376, 1175, 1154, 1087, 838 | 0.5 (d, J=6 Hz, 3H), 3.3-3.4 (m, 2H), 4 (m, 1H), 4.3 (m, 1H); 4.5 (s, 1H), 7.2 (m, 12H), 8 (m, 4H), |
| 32 | 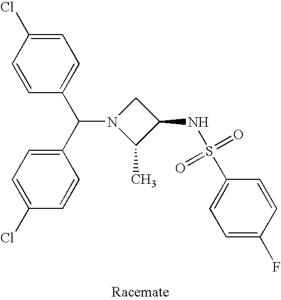 | 154-157 | 3238, 1591, 1490, 1325, 1157.1090, 802 | 0.6 (d, J=6 Hz, 3H), 2.3 (m, 1H), 2.9 (m, 1H), 3.4 (m, 2H); 4.2 (s, 1H), 4.75 (d, J=7.6 Hz, 1H), 7.1-7.25 (m, 12H), 7.8 (m, 2H) |
| 33 | 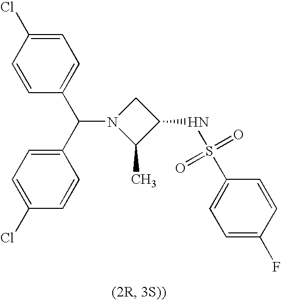 | 154-158 | 3254, 1593, 1492, 1154, 1329, 1090, 803 | 0.5 (d, J=6 Hz, 3H), 2.3 (m, 1H), 2.9 (m, 1H), 3.4 (m, 2H); 4.1 (s, 1H), 4.7 (d, J=8.9 Hz, 1H), 7.1-7.2 (m, 12H), 7.8 (m, 2H) |

Pharmacological Data:

I. In-vitro Determination of Affinity to CB1/CB2-Rezeptors

The affinity of the inventive substituted azetidine compounds to CB1/CB2 receptors is determined as described above. Some of the values obtained are given in the following table II:

TABLE II

| | | |
|---|---|---|
| | CB₁ Receptor | CB₂ Receptor |
| | Radioligand: | Radioligand: |
| Compound | [³H]−CP55940 | [³H]−CP55940 |

| according | % | | % | |
|---|---|---|---|---|
| to | Inhibition | | Inhibition | |
| Example | 10⁻⁶ M | Kᵢ (nM) | 10⁻⁶ M | Kᵢ (nM) |
| 1 | 60% | — | — | — |
| 5 | 59% | 264 | 4.6% | — |
| 6 | 66% | — | 13.5% | — |
| 14 | 82% | — | — | — |
| 15 | 66% | — | — | — |
| 18 | 95% | — | — | — |
| 25 | 87% | — | — | — |
| 33 | 71% | — | — | — |

II. In-vivo Bioassay System for Determination of Cannabinoid Activity

The determinination of cannabinoid activity in-vivo was determined as described above. Some of the values obtained are given in the following table III:

TABLE III

| | | | | | dosis administered 5 mg/kg | | | |
|---|---|---|---|---|---|---|---|---|
| Compound | dosis administered: | | | | i.v. prior to Win 55212-2 | | | |
| according | 5 mg/kg i.v. | | | | in a dose of 1.25 mg/kg i.v. | | | |
| to | Agonistic effect | | | | Antagonistic Effect | | | |
| example: | A | B | C. | D | A | B. | C | D |
| 1 | 5 | 0 | 0 | 0 | 46 | 100 | 47 | 20 |
| 5 | 2 | 0 | 0 | 0 | 77 | 100 | 71 | 57 |
| 6 | 0 | 0 | 0 | 0 | 32 | 32 | 51 | 46 |
| 14 | 16 | 0 | 0 | 0 | 12 | 71 | 14 | 9 |
| 15 | 14 | 0 | 0 | 0 | 72 | 100 | 57 | 71 |

i.v. intravenous
A: Hot-Plate test
B: Hypothermia
C: Catalepsy
D: Sedation

As can be seen from the values given in table III the inventive azetidine compounds act as cannabinoid receptor antagonists, particularly for the CB₁-receptors.

1. A substituted azetidine compound of general formula I, 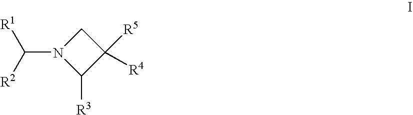
wherein
R¹ represents an at least mono-substituted phenyl group,
R² represents a saturated or unsaturated, optionally at least mono-substituted, optionally at least one heteroatom as ring member containing cycloaliphatic group, which may be condensed with an optionally at least mono-substituted mono- or polycyclic ring system, or an optionally at least mono-substituted aryl or heteroaryl group, which may be condensed with an optionally at least mono-substituted mono- or polycyclic ring system,
R³ represents a linear or branched, saturated or unsaturated, optionally at least mono-substituted aliphatic group, a saturated or unsaturated, optionally at least mono-substituted, optionally at least one heteroatom as ring member containing cycloaliphatic group, which may be condensed with a mono- or polycyclic ring system and/or which may be bonded via a linear or branched alkylene group, or an optionally at least mono-substituted aryl or heteroaryl group, which may be condensed with a mono- or polycyclic ring system and/or which may be bonded via a linear or branched alkylene group, with the proviso that R³ is bonded to the azetidine ring via a carbon atom,
R⁴ represents a hydrogen atom, a cyano group, a carboxy group, a linear or branched alkyl group, or an optionally at least mono-substituted aryl group,
R⁵ represents an —O—SO₂—R⁶-moiety, an —NH—CO—R⁷-moiety, an —NH₂-moiety, an —NH—SO₂—R⁸-moiety, an —NR⁹—SO₂—R¹⁰-moiety or an O—CO—R¹¹-moiety,
R⁶ represents a linear or branched, saturated or unsaturated, optionally at least mono-substituted aliphatic group, a saturated or unsaturated, optionally at least mono-substituted, optionally at least one heteroatom as ring member containing cycloaliphatic group, which may be condensed with a mono- or polycyclic ring system and/or which may be bonded via a linear or branched alkylene group, or an optionally at least mono-substituted aryl or heteroaryl group, which may be condensed with a mono- or polycyclic ring system and/or which may be bonded via a linear or branched alkylene group,
R⁷ represents a linear or branched, saturated or unsaturated, optionally at least mono-substituted aliphatic group, a saturated or unsaturated, optionally at least mono-substituted, optionally at least one heteroatom as ring member containing cycloaliphatic group, which may be condensed with a mono- or polycyclic ring system and/or which may be bonded via a linear or branched alkylene group, or an optionally at least mono-substituted aryl or heteroaryl group, which may be condensed with a mono- or polycyclic ring system and/or which may be bonded via a linear or branched alkylene group,
R⁸ represents a linear or branched, saturated or unsaturated, optionally at least mono-substituted aliphatic group, a saturated or unsaturated, optionally at least mono-substituted, optionally at least one heteroatom as ring member containing cycloaliphatic group, which may be condensed with a mono- or polycyclic ring system and/or which may be bonded via a linear or bridged alkylene group, and/or which may be bridged by a linear or branched alkylene group, or an optionally at least mono-substituted aryl or heteroaryl group, which may be condensed with a mono- or polycyclic ring system and/or which may be bonded via a linear or branched alkylene group,
R⁹ represents an —SO₂R¹²-moiety, a —CO—R¹³-moiety, a linear or branched, saturated or unsaturated, optionally at least mono-substituted aliphatic group, a saturated or unsaturated, optionally at least mono-substituted, optionally at least heteroatom as ring member containing cycloaliphatic group, which may be condensed with a mono- or polycyclic ring-system and/or which may be bonded by a linear or branched alkylene group and/or bridged by a linear or branched alkylene group, or an optionally at least mono-substituted aryl or heteroaryl group, which may be condensed with a mono- or polycyclic ring system and/or which may be bonded via an alkylene group,
R¹⁰ represents a linear or branched, saturated or unsaturated, optionally at least mono-substituted aliphatic group, a saturated or unsaturated, optionally at least mono-substituted, optionally at least one heteroatom as ring member containing cycloaliphatic group, which may be condensed with a mono- or polycyclic ring-system and/or which may be bonded via a linear or bridged alkylene group, and/or which may be bridged by a linear or branched alkylene group, or an optionally at least mono-substituted aryl or heteroaryl group, which may be condensed with a mono- or polycyclic ring system and/or which may be bonded via a linear or branched alkylene group,
R¹¹ represents a linear or branched, saturated or unsaturated, optionally at least mono-substituted aliphatic group, a saturated or unsaturated, optionally at least mono-substituted, optionally at least one heteroatom as ring member containing cycloaliphatic group, which may be condensed with a mono- or polycyclic ring-system and/or which may be bonded via a linear or bridged alkylene group, and/or which may be bridged by a linear or branched alkylene group, or an optionally at least mono-substituted aryl or heteroaryl group, which may be condensed with a mono- or polycyclic ring system and/or which may be bonded via a linear or branched alkylene group,
R¹²represents a linear or branched, saturated or unsaturated, optionally at least mono-substituted aliphatic group, a saturated or unsaturated, optionally at least mono-substituted, optionally at least one heteroatom as ring member containing cycloaliphatic group, which may be condensed with a mono- or polycyclic ring-system and/or which may be bonded via a linear or bridged alkylene group, and/or which may be bridged by a linear or branched alkylene group, or an optionally at least mono-substituted aryl or heteroaryl group, which may be condensed with a mono- or polycyclic ring system and/or which maybe bonded via a linear or branched alkylene group, and
R¹³ represents a linear or branched, saturated or unsaturated, optionally at least mono-substituted aliphatic group, a saturated or unsaturated, optionally at least mono-substituted, optionally at least one heteroatom as ring member containing cycloaliphatic group, which may be condensed with a mono- or polycyclic ring-system and/or which may be bonded via a linear or bridged alkylene group, and/or which may be bridged by a linear or branched alkylene group, or an optionally at least mono-substituted aryl or heteroaryl group, which may be condensed with a mono- or polycyclic ring system and/or which may be bonded via a linear or branched alkylene group,
optionally in the form of one of the stereoisomers, preferably enantiomers or diastereomers, a racemate or in the form of a mixture of at least two of the stereoisomers, preferably enantiomers and/or diastereomers, in any mixing ratio, or a corresponding N-oxide thereof, a corresponding salt thereof, or a corresponding solvate thereof,
with the proviso that compounds of general formula I, in which R¹ and R² each represent an unsubstituted phenyl group, R⁵ represents an —O—SO₂—R⁶-moiety and R⁶ represents a methyl group are excluded.

2. The compound according to claim 1, wherein
R¹ represents a phenyl group, said phenyl group being substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, —OCF₃, —CN and —COOH,
R² represents a phenyl group, said phenyl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, —OCF₃, —CN and —COOH,
R³ represents a linear or branched, unsubstituted C₁₋₆ alkyl group,
R⁴ represents a hydrogen atom, —CN, —COOH, a linear or branched, unsubstituted C₁₋₆ alkyl group, or a phenyl group, said phenyl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, —OCF₃, —CN and —COOH,
R⁵ represents an —O—SO₂—R⁶-moiety, an —NH—CO—R⁷-moiety, an —NH₂-moiety, an —NH—SO₂—R⁶ moiety, or an —NR⁹—SO₂—R¹⁰-moiety or an —O—C(═O)—R¹¹-moiety.
R⁶ represents a phenyl group, said phenyl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, —OCF₃, —CN and —COOH,
R⁷ represents a linear or branched C₁₋₅ alkyl group, said alkyl group being unsubstituted or substituted with 1, 2, 3, 4 or 5 substituents independently selected from the group consisting of F, Cl, Br, —OH, —SH, linear or branched C₁₋₆ alkoxy, —CF3 and —OCF₃—, a cyclopentyl or cyclohexyl group, said cyclopentyl or cyclohexyl group being unsubstituted or substituted with 1, 2, 3, 4 or 5 substituents independently selected from the group consisting of F, Cl, Br, —OH, —SH, linear or branched C₁₋₆ alkoxy, —CF₃ and —OCF₃; or a phenyl group, said phenyl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, —OCF₃, —CN and —COOH,
R⁸ represents a linear or branched C₁₋₅ alkyl group; an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, phenyl (optionally substituted with 1, 2, 3, 4 or 5 substituents independently selected from the group consisting of F, Cl, Br, OH, linear or branched C₁₋₆ alkyl and linear or branched C₁₋₆-alkoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alkoxy), —SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, ═O, —OCF₃, —CN and —COOH;
R⁹ represents an —SO₂—R¹²-moiety; a —(C═O)—R¹³-moiety, a linear or branched C₁₋₆ alkyl radical; or a phenyl radical, said phenyl radical being unsubstituted or substituted with 1, 2, 3, 4 or 5 substituents independently selected from the group consisting of F, Cl, Br, linear or branched C₁₋₆ alkyl, linear or branched C₁₋₆, alkoxy, —(C═O)—H, —OH, —CF₃, —OCF₃, —CN and —COOH and/or said phenyl radical being optionally bonded via a C₁₋₃ alkylene bridge,
R¹⁰ represents a linear or branched C₁₋₅ alkyl group; an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, phenyl (optionally substituted with 1, 2, 3, 4 or 5 substituents independently selected from the group consisting of F, Cl, Br, OH, linear or branched C₁₋₆ alkyl and linear or branched C₁₋₆-alkoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alkoxy), —SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, ═O, —OCF₃, —CN and —COOH;
R¹¹ represents a linear or branched C₁₋₅ alkyl group; an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazolo and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, phenyl (optionally substituted with 1, 2, 3, 4 or 5 substituents independently selected from the group consisting of F, Cl, Br, OH, linear or branched C₁₋₆ alkyl and linear or branched C₁₋₆-alkoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alkoxy), —SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₆ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, ═O, —OCF₃, —CN and —COOH;
R¹² represents a linear or branched C₁₋₅ alkyl group, an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, phenyl (optionally substituted with 1, 2, 3, 4 or 5 substituents independently selected from the group consisting of F, Cl, Br, OH, linear or branched C₁₋₆ alkyl and linear or branched C₁₋₆-alkoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alkoxy), SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, ═O, —OCF₃, —CN and —COOH; and
R¹³ represents a linear or branched C₁₋₅ alkyl group, an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, phenyl (optionally substituted with 1, 2, 3, 4 or 5 substituents independently selected from the group consisting of F, Cl, Br, OH, linear or branched C₁₋₆ alkyl and linear or branched C₁₋₆-alkoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alkoxy), —SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, ═O, —OCF₃, —CN and—COOH;
optionally in the form of one of the stereoisomers, preferably enantiomers or diastereomers, a racemate or in the form of a mixture of at least two of the stereoisomers, preferably enantiomers and/or diastereomers, in any mixing ratio, or a corresponding N-oxide thereof, a corresponding salt thereof, or a corresponding solvate thereof.

3. The compound according to claim 1 of the formula: 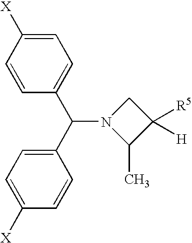
wherein
X represents a halogen atom, preferably a chlorine atom,
R⁵ represents an —O—SO₂—R⁶ moiety, an —NH—CO—R⁷-moiety, an —NH₂-moiety, an —NH—SO₂—R⁸ moiety, or an —NR⁹—SO₂—R¹⁰-moiety or an —O—C(═O)R¹¹-moiety,
R⁶ represents a phenyl group, said phenyl group being unsubstituted or substituted with 1, 2, 3,4 or 5 substituents independently selected from the group consisting of F, Cl, Br, linear or branched C¹⁻⁶ alkyl, linear or branched C₁₋₆ alkoxy, —(C═O)—H, —OH, —CF₃, —OCF₃, —CN and —COOH,
R⁷ represents a linear or branched C₁₋₅ alkyl group, said alkyl group being unsubstituted or substituted with 1, 2, 3, 4 or 5 substituents independently selected from the group consisting of F, Cl, Br, —OH, —SH, linear or branched C₁₋₆ alkoxy, —CF₃ and —OCF₃; a cyclopentyl or cyclohexyl group, said cyclopentyl or cyclohexyl group being unsubstituted or substituted with 1, 2, 3, 4 or 5 substituents independently selected from the group consisting of F, Cl, Br, —OH, —SH, linear or branched C₁₋₆ alkoxy, —CF₃ and —OCF₃; or a phenyl group, said phenyl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, —OCF₃, —CN and —COOH,
R⁸ represents a linear or branched C₁₋₅ alkyl group; an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, phenyl (optionally substituted with 1, 2, 3, 4 or 5 substituents independently selected from the group consisting of F, Cl, Br, OH, linear or branched C₁₋₆ alkyl and linear or branched C₁₋₆-alkoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alkoxy), —SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, ═O, —OCF₃, —CN and —COOH;
R⁹ represents an —SO₂R¹²-moiety; a —(C═O)—R¹³-moiety; a linear or branched C₁₋₆ alkyl radical; or a phenyl radical, said phenyl radical being unsubstituted or substituted with 1, 2, 3, 4 or 5 substituents independently selected from the group consisting of F, Cl, Br, linear or branched C₁₋₆ alkyl, linear or branched C₁₋₆-alkoxy, —(C═O)—H, —OH, —CF₃, —OCF₃, —CN and —COOH and/or said phenyl radical being optionally bonded via a C₁₋₃-alkylene bridge,
R¹⁰ represents a linear or branched C₁₋₆ alkyl group; an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, phenyl (optionally substituted with 1, 2, 3, 4 or 5 substituents independently selected from the group consisting of F, Cl, Br, OH, linear or branched C₁₋₆ alkyl and linear or branched C₁₋₆ alkoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alkoxy), —SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, ═O, —OCF₃, —CN and —COOH;
R¹¹ represents a linear or branched C₁₋₅ alkyl group; an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, phenyl (optionally substituted with 1, 2, 3, 4 or 5 substituents independently selected from the group consisting of F, Cl, Br, OH, linear or branched C₁₋₆ alkyl and linear or branched C₁₋₆-alkoxy), phenoxy (optionally substituted with 1, 2, 3, 4 or 5 substituents independently selected from the group consisting of F, Cl, Br, OH, linear or branched C₁₋₆ alkyl and linear or branched C₁₋₆ alkoxy), SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, ═O, —OCF₃, —CN and —COOH;
R¹² represents a linear or branched C₁₋₅ alkyl group, an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, phenyl (optionally substituted with 1, 2, 3, 4 or 5 substituents independently selected from the group consisting of F, Cl, Br, OH, linear or branched C₁₋₆ alkyl and linear or branched C₁₋₆-alkoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alkoxy), —SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, ═O, —OCF₃, —CN and —COOH; and
R¹³ represents a linear or branched C₁₋₆ alkyl group, an aryl or heteroaryl group selected from the group consisting of phenyl, naphthyl, thlenyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, phenyl (optionally substituted with 1, 2, 3, 4 or 5 substituents independently selected from the group consisting of F, Cl, Br, OH, linear or branched C₁₋₆ alkyl and linear or branched C₁₋₆-alkoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alkoxy), —SO₂—CH₃, —(C═O)H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, ═O, —OCF₃, —CN and —COOH;
optionally in the form of one of the stereoisomers, preferably enantiomers or diastereomers, a racemate or in the form of a mixture of at least two of the stereoisomers, preferably enantiomers and/or diastereomers, in any mixing ratio, or a corresponding N-oxide thereof, a corresponding salt thereof, or a corresponding solvate thereof.

4. A compound according to claim 1 of general formula I, 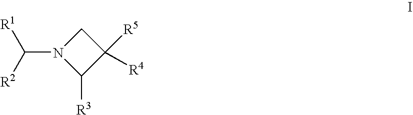
wherein
R¹ represents a phenyl group, which is mono-substituted with a halogen atom, preferably a chlorine atom, in the 4-position of the phenyl ring,
R² represents a phenyl group, which is mono-substituted with a halogen atom, preferably a chlorine atom, in the 4-position of the phenyl ring,
R³ represents a linear or branched, unsubstituted C₁₋₆ alkyl group, preferably a methyl group,
R⁴ represents a hydrogen atom,
R⁵ represents an —O—SO₂R⁶-moiety, an —NH—CO—R⁷-moiety, an —NH₂-moiety, an —NH—SO₂—R⁸ moiety, or an —NR⁹—SO₂—R¹⁻-moiety,
R⁶ represents a phenyl ring, which is optionally substituted with one or more halogen atoms, preferably one or more fluorine and/or one or more chlorine atoms,
R⁷ represents a linear or branched C₁₋₆ alkyl group, a linear or branched C₁₋₆ alkyl group, which is at least partially fluorinated, a C₃₋₆ cycloalkyl group, or a phenyl group, which is optionally substituted with one or more halogen atoms, preferably one or more fluorine atoms,
R⁸ represents a linear or branched C₁₋₆ alkyl group,
a phenyl group, which is optionally substituted with one or more substituents independently selected from the group consisting of a fluorine atom, a chlorine atom, an unsubstituted phenyl group, a formyl group, a methylsulfonyl group, a benzyl group and a phenoxy group, which is optionally mono-substituted by a bromine atom in its 4-position,
a naphthyl group, which may be bonded via a methylene or ethylene group,
a Benzo[b]thiophene group, which is optionally substituted with one or more methyl groups and/or one or more chlorine atoms,
a pyrazole group, which is optionally substituted with one or more substituents independently selected from the group consisting of a methyl group, an ethyl group and a phenyl group,
an imidazo[2,1-b]thiazole group, which is optionally substituted with one or more chlorine atoms,
a thienyl group, a furyl group, a 2,1,3-Benzothiadiazole group, a 7,7-Dimethyl-2-oxo-bicyclo-[2.2.1]-hept-1-yl group, or a benzyl group,
R⁹ represents a C₁₋₅ alkyl group, preferably a methyl group, a phenyl group, which is optionally substituted with one or more fluorine atoms and/or one or more chlorine atoms, a benzyl group, wherein the ring is optionally substituted with one or more fluorine atoms and/or one or more chlorine atoms, or a —SO₂—R¹²-moiety,
R¹⁰ represents a phenyl group, which is optionally substituted with one or more fluorine atoms and/or one or more chlorine atoms, and
R¹²represents a C₁₋₅ alkyl group, preferably a methyl group, or a phenyl group, which is optionally substituted with one or more fluorine atoms and/or one or more chlorine atoms,
optionally in the form of one of the stereoisomers, preferably enantiomers or diastereomers, a racemate or in the form of a mixture of at least two of the stereoisomers, preferably enantiomers and/or diastereomers, in any mixing ratio, or a corresponding N-oxide thereof, a corresponding salt thereof, or a corresponding solvate thereof.

5. A compound according to claim 1 selected from the group consisting of
4-Fluoro-benzenesutfonic acid 1-[trans-bis-(4-chloro-phenyl)-methyl)-2-methyl-azetidin-3-yl ester,
N-((2S,3R)-1-[Bis-(4-chloro-phenyl)-methyl]-2-methyl-azetidin-3-yl]-2,2,2-trifluoro-acetamide,
(2S,3R)-1-[Bis(4-chloro-phenyl)-methyl]-2-methyl-azetidin-3-ylamine,
Hexanoic acid {1-[bis-(4-chloro-phenyl)-methyl]-2-methyl-azetidin-3-yl}-amide,
N-{(2S,3R)-1-[Bis-(4-chloro-phenyl)methyl]-2-methyl-azetidin-3-yl}-4-fluoro-benzenesulfonamide,
Thiophene-2-sulfonic acid {(2S,3R)-1-[bis-{4-chloro-phenyl}-methyl]-2-methyl-azetidin-3-yl}-amide,
Cyclohexanecarboxylic acid {(2S,3R)-1-[bis-(4-chloro-phenyl)-methyl]-2 methyl-azetidin-3-yl}-amide,
Butane-1-sulfonic acid {(2S,3R)-1-[bis-(4-chloro-phenyl)-methyl]-2-methyl-azetidin-3-yl}-amide,
N-{(2S,3R)-1-[Bis-(4-chloro-phenyl)-methyl]-2-methyl-azetidin-3-yl}-3,5-difluoro-benzamide,
Naphthalene-2-sulfonic acid {trans-1-[bis-(4-chloro-phenyl)-methyl]-2methyl-azetidin-3-yl}-amide,
Biphenyl-4-sulfonic acid {trans-1-[bis-(4-chloro-phenyl)-methyl]-2-methyl-azetidin-3-yl}-amide,
4-Acetyl-N-{trans-1-[bis-(4-chloro-phenyl)-methyl]-2-methyl-azetidin-3-yl}-benzenesulfonamide,
N-{trans-1-[Bis-(4-chloro-phenyl)-methyl]-2-methyl-azetidin-3-yl}-4-(4-bromo-phenoxy)-benzenesulfonamide,
N-{trans-1-[Bis-(4-chloro-phenyl)-methyl]-2-methyl-azetidin-3-yl)-4-methylsulfonyl-benzenesulfonamide,
2,1,3-Benzothiadiazole-4-sulfonic acid {trans-1-[bis-(4-chloro-phenyl)-methyl]-2-methyl-azetidin-3-yl}-amide,
5-Chloro-3-methyl-benzo[b]thiophene-2-sulfonic acid {trans-1-[bis-(4-chloro-phenyl)-methyl]-2methyl-azetidin-3-yl}-amide,
6-Chloro-imidazo[2,1-b]thiazole-5-sulfonic acid {trans-1-[bis-(4-chloro-phenyl)-methyl]-2-methyl-azetidin-3-yl}-amide,
N-{trans-1-[Bis-(4-chloro-phenyl)-methyl}-2-methyl-azetidin-3-yl)-3,5-dichloro-benzenesulfonamide,
2-Naphthalene-1-yl-ethanesulfonic acid {trans-1-[bis-(4-chloro-phehyl)-methyl]-2-methyl-azetidin-3-yl}-amide,
N-{trans-1-[Bis-(4-chloro-phenyl)-methyl]-2-methyl-azetidin-3-yl}-phenyl-methylsulfonamide,
N-trans-1-[Bis-(4-chloro-phenyl)-methyl]-2 methyl-azetidin-3-yl}-(7,7-dimethyl-2-oxo-bicyclo[2.2.1]hept-1-yl)-methylsulfonamide,
Naphthalene-1-sulfonic acid {trans-1-[bis-(4-chloro-phenyl)-methyl]-2-methyl-azetidin-3-yl}-amide,
N-{trans-1-[Bis-{4-chloro-phenyl}methyl]-2-methyl-azetidin-3-yl}-4-phenoxy-benzonesulfonamide,
1,3,5-Trimethyl-1H-pyrazole-4-sulfonic acid {trans-1-[bis-(4-chloro-phenyl)-methyl]-2-methyl-azetidin 3-yl}-amide,
Benzo[b]thiophene-sulfonic acid {trans-1-[bis-(4-chloro-phenyl)-methyl]-2-methyl-azetidin-3-yl-amide,
5-Methyl-1-phenyl-1H-pyrazole-sulfonic acid {trans-1-[bis-(4-chloro-phenyl)-methyl]-2-methyl-azetidin-3-yl}-amide,
N-{trans-1-Bis-(4-chloro-phenyl)-methyl]-2-methyl-azetidin-3-yl}-N-methyl-4-fluoro-benzenesulfonamide,
N-{trans-1-[Bis-(4-chloro-phenyl)-methyl]-2-methyl-azetidin-3-yl}-N-(4-fluoro-benzyl)-4-fluoro-benzenesulfonamide,
N-{trans-1-[Bis-(4-chlorophenyl)-methyl]-2-methyl-azetidin-3-yl}-N-propyl-4-fluoro-benzenesulfonamide,
N-{trans-1-[Bis-(4-chloro-phenyl)-methyl]-2-methyl-azetidin-3-yl}-N-(methylsulphonyl)-4-fluoro-benzenesulfonamide,
N-{trans-1-[Bis-(4-chloro-phenyl)-methyl]-2 methyl-azetidin-3-yl}-N-bis(4-fluoro-benzenesulfonamide),
N-{(trans-1-(Bis-(4-chloro-phenyl)-methy[ ]-2-methyl-azetidin-3-yl)-4-fluoro-benzenesulfonamide, and
N-{(2R,3S)-1-[Bis-(4-chloro-phenyl)-methyl]-2-methyl-azetidin-3-yl}-4-fluoro-benzenesulfonamide;
optionally in the form of a corresponding N-oxide, a corresponding salt or a corresponding solvate.

6. A process for the preparation of substituted azetidine compounds according to claim 1, comprising reacting at least one compound of general formula II 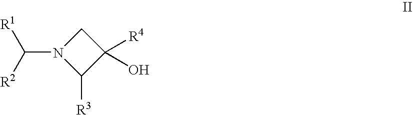
wherein R¹ to R⁴ have the meaning as in claim 1 with at least one compound of general formula X¹—SO₂—R⁶ or X²—CO—R¹¹, wherein R⁶ and R¹¹ have the meaning as in claim 1 and X¹ and X² are leaving groups, in a reaction medium, optionally in the presence of at least one base, to yield at least one compound of general formula I according to claim 1, wherein R⁵represents an —O—SO₂—R⁶ or a -Q-CO—R¹¹ moiety, and optionally purifying and/or optionally isolating said compound(s), and optionally at least one of these afore mentioned compounds, wherein R⁵ represents a —O—SO₂—R⁶ or an —O—CO—R¹¹ moiety is reacted with ammonia, to yield a compound of general formula I according to claim 1, wherein R⁵ represents an —NH₂-moiety, and optionally purifying and/or optionally isolating said compound(s),
and optionally at least one of these afore mentioned compounds, wherein R⁵ represents an —NH₂-moiety, is reacted with at least one compound of general formula X³—COR⁷, X⁴—SO₂—R⁸ or X⁵—SO₂—R¹⁰, wherein R⁷, R⁸ and R¹⁰ have the meaning as in claim 1 and X³, X⁴ and X⁵ represent leaving groups, in a reaction medium, optionally in the presence of at least one base, to yield a compound of general formula I according to claim 1, wherein R⁵ represents an —NH—CO—R⁷-moiety, an —NH—SO₂—R⁸-moiety, or an —NR⁹—SO₂—R¹⁰-moiety with R⁹ representing a hydrogen atom, and optionally purifying and/or optionally isolating said compound(s),
and optionally at least one compound of general formula I, wherein R⁶ represents an —NR⁹—SO₂—R¹⁰-moiety with R⁹ representing a hydrogen atom is reacted with at least one compound of general formula X⁶—R⁹, wherein R⁹ has the meaning as in claim 1 with the exception of a hydrogen atom, and X⁶ represents a leaving group, to yield at least one compound of general formula I according to claim 1, wherein R⁵ represents an —NR⁹—SO₂R¹⁰-moiety, and optionally purifying and/or optionally isolating, said compound(s),
or, oxidizing at least one compound of general formula III, 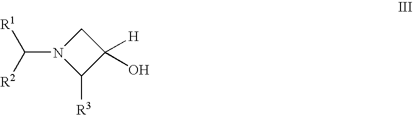
wherein R¹—R³ have the meaning according to claim 1 to yield at least one compound of general formula IV, 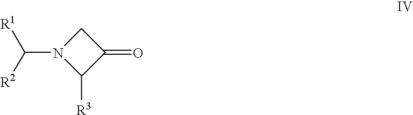
wherein R¹—R³ have the meaning as in claim 1, which is optionally purified and/or optionally isolated, and reacting the compound with at least one compound of general formula R^{5a}H, wherein R^{5a} represents an —NH₂-moiety or an —NHR⁹-moiety, wherein R⁹ has the meaning given above, wherein the resulting compound is optionally purified and/or optionally isolated and optionally reacted with at least one compound of general formula X³—CO—R⁷, X⁴SO₂—R⁸ or X⁵—SO₂—R¹⁰, wherein R⁷, R⁸ and R¹⁰ have the meanings given above and X³, X⁴ and X⁵ represent leaving groups, in a reaction medium, optionally in the presence of at least one base, to yield a compound of general formula I according to claim 1, wherein R⁵ represents an —NH₂-moiety, an —NH—CO—R⁷-moiety, an —NH—SO₂—R⁸-moiety, or an —NR⁹—SO₂R¹⁰-moiety, which is optionally purified and/or isolated.

7. A process for the preparation of a substituted azetidine compound according to claim 1, comprising reacting at least one compound of general formula V, 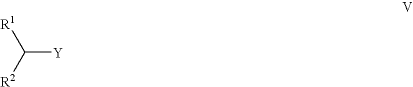
wherein Y represents a halogen atom, preferably a chlorine atom or a bromine atom, and R¹ and R² have the meaning according to claim 1, with at least one compound of general formula VI, 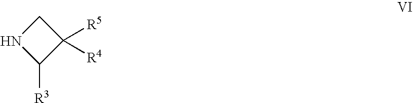
optionally in the form of a salt, wherein R³, R⁴ and R⁵ have the meaning according to claim 1, in a suitable reaction medium, optionally in the presence of a base, and the resulting azetidine compound(s) is/are optionally purified and/or optionally isolated.

8. A medicament comprising at least one substituted azetidine compound of general formula I, 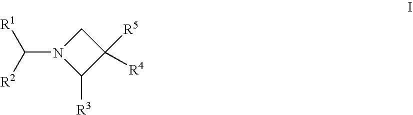
wherein
R¹ represents an optionally at least mono-substituted phenyl group,
R² represents a saturated or unsaturated, optionally at least mono-substituted, optionally at least one heteroatom as ring member containing cycloaliphatic group, which may be condensed with an optionally at least mono-substituted mono- or polycyclic ring system, or an optionally at least mono-substituted aryl or heteroaryl group, which may be condensed with an optionally at least mono-substituted mono- or polycyclic ring system,
R³ represents a linear or branched, saturated or unsaturated, optionally at least mono-substituted aliphatic group, a saturated or unsaturated, optionally at least mono-substituted, optionally at least one heteroatom as ring member containing cycloaliphatic group, which may be condensed with a mono- or polycyclic ring-system and/or which may be bonded via a linear or branched alkylene group, or an optionally at least mono-substituted aryl or heteroaryl group, which may be condensed with a mono- or polycyclic ring system and/or which may be bonded via a linear or branched alkylene group, with the proviso that R³ is bonded to the azetidine ring via a carbon atom,
R⁴ represents a hydrogen atom, a cyano group, a carboxy group, a linear or branched alkyl group, or an optionally at least mono-substituted aryl group,
R⁵ represents an —O—SO₂—R⁶-moiety, an —NH—CO—R⁷-moiety, an —NH₂-moiety, an —NH—SO₂—R⁸ moiety, an —NR⁹—SO₂—R¹⁰-moiety or an O—CO—R¹¹-moiety,
R⁶ represents a linear or branched, saturated or unsaturated, optionally at least mono-substituted aliphatic group, a saturated or unsaturated, optionally at least mono-substituted, optionally at least one heteroatom as ring member containing cycloaliphatic group, which may be condensed with a mono- or polycyclic ring-system and/or which may be bonded via a linear or branched alkylene group, or an optionally at least mono-substituted aryl or heteroaryl group, which may be condensed with a mono- or polycyclic ring system and/or which may be bonded via a linear or branched alkylene group,
R⁷ represents a linear or branched, saturated or unsaturated, optionally at least mono-substituted aliphatic group, a saturated or unsaturated, optionally at least mono-substituted, optionally at least one heteroatom as ring member containing cycloaliphatic group, which may be condensed with a mono- or polycyclic ring system and/or which may be bonded via a linear or branched alkylene group, or an optionally at least mono-substituted aryl or heteroaryl group, which may be condensed with a mono- or polycyclic ring system and/or which may be bonded via a linear or branched alkylene group,
R⁸ represents a linear or branched, saturated or unsaturated, optionally at least mono-substituted aliphatic group, a saturated or unsaturated, optionally at least mono-substituted, optionally at least one heteroatom as ring member containing cycloaliphatic group, which may be condensed with a mono- or polycyclic ring system and/or which may be bonded via a linear or bridged alkylene group, and/or which may be bridged by a linear or branched alkylene group, or an optionally at least mono-substituted aryl or heteroaryl group, which may be condensed with a mono- or polycyclic ring system and/or which may be bonded via a linear or branched alkylene group,
R⁹ represents an —SO₂R¹²-moiety, a —CO—R¹³-moiety, a linear or branched, saturated or unsaturated, optionally at least mono-substituted aliphatic group, a saturated or unsaturated, optionally at least mono-substituted, optionally at least heteroatom as ring member containing cycloaliphatic group, which may be condensed with a mono- or polycyclic ring-system and/or which may be bonded by a linear or branched alkylene group and/or bridged by a linear or branched alkylene group, or an optionally at least mono-substituted aryl or heteroaryl group, which may be condensed with a mono- or polycyclic ring system and/or which may be bonded via an alkylene group,
R¹⁰ represents a linear or branched, saturated or unsaturated, optionally at least mono-substituted aliphatic group, a saturated or unsaturated, optionally at least mono-substituted, optionally at least one heteroatom as ring member containing cycloaliphatic group, which may be condensed with a mono- or polycyclic ring system and/or which may be bonded via a linear or bridged alkylene group, and/or which maybe bridged by a linear or branched alkylene group, or an optionally at least mono-substituted aryl or heteroaryl group, which may be condensed with a mono- or polycyclic ring system and/or which may be bonded via a linear or branched alkylene group,
R¹¹ represents a linear or branched, saturated or unsaturated, optionally at least mono-substituted aliphatic group, a saturated or unsaturated, optionally at least mono-substituted, optionally at least one heteroatom as ring member containing cycloaliphatic group, which may be condensed with a mono- or polycyclic ring system and/or which maybe bonded via a linear or bridged alkylene group, and/or which may be bridged by a linear or branched alkylene group, or an optionally at least mono-substituted aryl or heteroaryl group, which may be condensed with a mono- or polycyclic ring system and/or which may be bonded via a linear or branched alkylene group,
R¹² represents a linear or branched, saturated or unsaturated, optionally at least mono-substituted aliphatic group, a saturated or unsaturated, optionally at least mono-substituted, optionally at least one heteroatom as ring member containing cycloaliphatic group, which may be condensed with a mono- or polycyclic ring system and/or which may be bonded via a linear or bridged alkylene group, and/or which may be bridged by a linear or branched alkylene group, or an optionally at least mono-substituted aryl or heteroaryl group, which may be condensed with a mono- or polycyclic ring system and/or which may be bonded via a linear or branched alkylene group, and
R¹³ represents a linear or branched, saturated or unsaturated, optionally at least mono-substituted aliphatic group, a saturated or unsaturated, optionally at least mono-substituted, optionally at least one heteroatom as ring member containing cycloaliphatic group, which may be condensed with a mono- or polycyclic ring system and/or which may be bonded via a linear or bridged alkylene group, and/or which may be bridged by a linear or branched alkylene group, or an optionally at least mono-substituted aryl or heteroaryl group, which may be condensed with a mono- or polycyclic ring system and/or which may be bonded via a linear or branched alkylene group,
optionally in the form of one of the stereoisomers, preferably enantiomers or diastereomers, a racemate or in the form of a mixture of at least two of the stereoisomers, preferably enantiomers and/or diastereomers, in any mixing ratio, or a corresponding N-oxide thereof, a corresponding salt thereof, or a corresponding solvate thereof,
and optionally one or more pharmaceutically acceptable excipients.

9. The medicament according to claim 8, wherein R¹ represents a phenyl group, which is optionally substituted by one or more substituents independently selected from the group consisting of a halogen atom, a linear or branched C₁₋₆-alkyl group, a linear or branched C₁₋₆ alkoxy group, a formyl group, a hydroxy group, a trifluoromethyl group, a trifluoromethoxy group, a —CO—C₁₋₆-alkyl group, a cyano group, a carboxy group, a —CO—O—C₁₋₆-alkyl group, a —CO—NR^{A}R^{B}-moiety, a —CO—NH—NR^{C}R^{D}-moiety, an —S—C₁₋₆-alkyl group, an —SO—C₁₋₈-alkyl group, an —SO—C₁₋₆-alkyl group, a —C₁₋₆-alkylene-S—C₁₋₆-alkyl group, a —C₁₋₈-alkylene-SO—C₁₋₆-alkyl group, a C₁₋₆-alkylene-SO₂—C₁₋₆-alkyl group, a C₁₋₆-alkyl group substituted by one or more hydroxy groups and a C₁₋₆-alkylene-NR^{E}R^{F} group,
whereby R^{A}, R^{B}, identical or different, represent hydrogen or a C₁₋₆-alkyl group, or R^{A} and R^{B} together with the bridging nitrogen atom form a saturated, mono- or bicyclic, 3-10 membered heterocyclic ring system, which may be at least mono-substituted by one or more, identical or different, C₁₋₆ alkyl groups and/or which may contain at least one further heteroatom selected from the group consisting of nitrogen, oxygen and sulphur as a ring member,
R^{C}, R^{D}, identical or different, represent a hydrogen atom, a C₁₋₆-alkyl group, a —CO—O—C₁₋₆-alkyl group, a C₃₋₈-cycloalkyl group, a C₁₋₆-alkylene-C₃₋₈-cycloalkyl group, C₁₋₆-alkylene-O—C₁₋₆-alkyl group or a C₁₋₆-alkyl group substituted with one or more hydroxy groups, or R^{C}, R^{D} together with the bridging nitrogen atom form a saturated, mono- or bicyclic, 3-10 membered heterocyclic ring system, which may be at least mono-substituted by one or more substituents independently selected from the group consisting of C₁₋₆ alkyl group, a —CO—C₁₋₆-alkyl group, a —CO—O—C₁₋₈-alkyl group, a —CO—NH—C₁₋₆-alkyl group, a —CS—NH—C₁₋₆-alkyl group, an oxo group, a C₁₋₆-alkyl group substituted with one or more hydroxy groups, a C₁₋₆-alkylene-O—C₁₋₆-alkyl group and a —CO—NH₂ group and/or which may contain at least one further heteroatom selected from the group consisting of nitrogen, oxygen and sulphur as a ring member, and
wherein R^{E}, R^{F}, identical or different, represent hydrogen or a C₁₋₆-alkyl group, or R^{E} and R^{F} together with the bridging nitrogen atom form a saturated, mono- or bicyclic, 3-10 membered heterocyclic ring system, which may be at least mono-substituted by one or more, identical or different C₁₋₆ alkyl groups and/or which may contain at least one further heteroatom selected from the group consisting of nitrogen, oxygen and sulphur as a ring member, and
preferably R¹ represents a phenyl group, which is optionally substituted by one or more substituents independently selected from the group consisting of a fluorine atom, a chlorine atom, a bromine atom, a linear or branched C₁₋₆-alkyl group, a linear or branched C₁₋₆ alkoxy group, a formyl group, a hydroxy group, a trifluoromethyl group, a trifluoromethoxy group, a cyano group and a carboxy group, more preferably R¹ represents a phenyl group, which is optionally substituted by one or more substituents independently selected from the group consisting of a fluorine atom, a chlorine atom, a bromine atom, a methyl group, a methoxy group, a trifluoromethyl group and a trifluoromethoxy group, most preferably R¹ represents a phenyl group, which is substituted by a chlorine atom in the 4-position.

10. The medicament according to claim 8, wherein R² represents a saturated or unsaturated, optionally at least mono-substituted, optionally at least heteroatom as ring member containing C₃₋₈ cycloaliphatic group, which may be condensed with an optionally at least mono-substituted mono- or polycyclic ring system, or an optionally at least mono-substituted, 5- or 6-membered aryl or heteroaryl group, which may be condensed with an optionally at least monosubstituted mono- or polycyclic ring system, preferably R² represents a phenyl group, which is optionally substituted by one or more substituents independently selected from the group consisting of a halogen atom, a linear or branched C₁₋₆-alkyl group, a linear or branched C₁₋₆ alkoxy group, a formyl group, a hydroxy group, a trifluoromethyl group, a trifluoromethoxy group, a —CO—C₁₋₆-alkyl group, a cyano group, a carboxy group, a —CO—O—C₁₋₆-alkyl group, a —CO—NR^{A}R^{B}-moiety, a —CO—NH—NR^{C}R^{D}-moiety, an —S—C₁₋₆-alkyl group, an —SO—C₁₋₆-alkyl group, an —SO₂—C₁₋₆-alkyl group, a —C₁₋₆-alkylene-S—C₁₋₆-alkyl group, a —C₁₋₆-alkylene-SO₂—C₁₋₆-alkyl group, a —C₁₋₆-alkylene-SO₂—C₁₋₆-alkyl group, a C₁₋₆-alkyl group substituted by one or more hydroxy groups and a —C₁₋₆-alkylene-NR^{E}R^{F} group,
whereby R^{A}, R^{B}, identical or different, represent hydrogen or a C₁₋₆-alkyl group, or R^{A} and R^{B} together with the bridging nitrogen atom form a saturated, mono- or bicyclic, 3-10 membered heterocyclic ring system, which may be at least mono-substituted by one or more, identical or different, C₁₋₆ alkyl groups and/or which may contain at least one further heteroatom selected from the group consisting of nitrogen, oxygen and sulphur as a ring member,
R^{C}, R^{D}, identical or different, represent a hydrogen atom, a C₁₋₆-alkyl group, a —CO—O—C₁₋₆-alkyl group, a C₃₋₈-cycloalkyl group, a C₁₋₆-alkylene-C₃₋₈-cycloalkyl group, C₁₋₆-alkylene-O—C₁₋₆-alkyl group or a C₁₋₆-alkyl group substituted with one or more hydroxy groups, or R^{C}, R^{D} together with the bridging nitrogen atom form a saturated, mono- or bicyclic, 3-10 membered heterocyclic ring system, which may be at least mono-substituted by one or more substituents independently selected from the group consisting of C₁₋₆ alkyl group, a —CO—C₁₋₆-alkyl group, a —CO—O—C₁₋₆-alkyl group, a —CO—NH—C₁₋₆-alkyl group, a —CS—NH—C₁₋₆-alkyl group, an oxo group, a C₁₋₆-alkyl group substituted with one or more hydroxy groups, a C₁₋₆-alkylene-O—C₁₋₆-alkyl group and a —CO—NH₂ group and/or which may contain at least one further heteroatom selected from the group consisting of nitrogen, oxygen and sulphur as a ring member, and
wherein R^{E}, R^{F}, identical or different, represent hydrogen or a C₁₋₆-alkyl group, or R^{E} and R^{F} together with the bridging nitrogen atom form a saturated, mono- or bicyclic, 3-10 membered heterocyclic ring system, which may be at least mono-substituted by one or more, identical or different C₁₋₆ alkyl groups and/or which may contain at least one further heteroatom selected from the group consisting of nitrogen, oxygen and sulphur as a ring member,
more preferably R² represents a phenyl group, which is optionally substituted by one or more substituents independently selected from the group consisting of a fluorine atom, a chlorine atom, a bromine atom, a methyl group, a methoxy group, a trifluoromethyl group and a trifluoromethoxy group, most preferably R² represents a phenyl group, which is substituted by a chlorine atom in the 4-position.

11. The medicament according to claim 8, wherein R³ represents a linear or branched, saturated or unsaturated, optionally at least mono-substituted C₁₋₁₀-aliphatic group, a saturated or unsaturated, optionally at least mono-substituted, optionally at least one heteroatom as ring member containing C₃₋₈-cycloaliphatic group, which may be condensed with a mono- or polycyclic ring-system and/or which may be bonded via a linear or branched C₁₋₆-alkylene group, or an optionally at least mono-substituted, 5- or 6-membered aryl or heteroaryl group, which may be condensed with a mono- or polycyclic ring system and/or which may be bonded via a linear or branched C₁₋₆-alkylene group, preferably R³ represents a linear or branched, optionally at least mono-substituted C₁₋₁₀-alkyl group, or an optionally at least mono-substituted, 5- or 6-membered aryl or heteroaryl group, which may be condensed with a mono- or polycyclic ring system and/or which may be bonded via a linear or branched C₁₋₆-alkylene group, more preferably R³ represents a linear or branched, unsubstituted C₁₋₁₀-alkyl group, most preferably R³ represents a methyl group.

12. The medicament according to claim 8, wherein R⁴ represents a hydrogen atom, a cyano group, a carboxy group, a linear or branched C₁₋₁₀-alkyl group, or an optionally at least mono-substituted, 5- or 6-membered aryl group, preferably R⁴ represents a hydrogen atom, a linear or branched C₁₋₃-alkyl group, or an optionally at least mono-substituted phenyl group, more preferably R⁴ represents a hydrogen atom or a linear or branched C₁₋₃-alkyl group, most preferably R⁴ represents a hydrogen atom.

13. The medicament according to claim 8, wherein R⁵ represents an —O—SO₂—R⁶-moiety, an —NH—CO—R⁷-moiety, an —NH²-moiety, an —NH—SO₂R⁸ moiety or an —NR⁹—SO₂—R¹⁰-moiety, preferably R⁵ represents an —O—SO₂—R⁶-moiety, an —NH—SO₂—R⁸ moiety or an —NR⁹—SO₂—R¹⁰-moiety.

14. The medicament according to claim 8, wherein R⁶ represents a linear or branched, saturated or unsaturated, optionally at least mono-substituted C₁₋₁₀ aliphatic group, a saturated or unsaturated, optionally at least mono-substituted, optionally at least one heteroatom as ring member containing C₃₋₈-cycloaliphatic group, which may be condensed with a mono- or polycyclic ring-system and/or which may be bonded via a linear or branched C₁₋₆-alkylene group, or an optionally at least mono-substituted, 5- or 6-membered aryl or heteroaryl group, which may be condensed with a mono- or polycyclic ring system and/or which may be bonded via a linear or branched C₁₋₆-alkylene group, preferably R⁶ represents an optionally at least mono-substituted C₃₋₈-cycloaliphatic group or an optionally at least mono-substituted phenyl group, wherein the respective substituents are independently selected from the group consisting of a fluorine atom, a chlorine atom, a bromine atom, a linear or branched C₁₋₆-alkyl group, a linear or branched C₁₋₆ alkoxy group, a formyl group, a hydroxy group, a trifluoromethyl group, a trifluoromethoxy group, a cyano group and a carboxy group, more preferably R⁵ represents a phenyl group, which is optionally substituted by one or more substituents independently selected from the group consisting of a fluorine atom, a chlorine atom, a bromine atom, a methyl group, a methoxy group, a trifluoromethyl group and a trifluoromethoxy group.

15. The medicament according to claim 8, wherein R⁷ represents a linear or branched, saturated or unsaturated, optionally at least mono-substituted C₁₋₁₀-aliphatic group, a saturated or unsaturated, optionally at least mono-substituted, optionally at least one heteroatom as ring member containing C₃₋₈-cycloaliphatic group, which may be condensed with a mono- or polycyclic ring-system and/or which may be bonded via a linear or branched C₁₋₆-alkylene group, or an optionally at least mono-substituted aryl or heteroaryl group, which may be condensed with a mono- or polycyclic ring system and/or which may be bonded via a linear or branched C₁₋₆-alkylene group, preferably R⁷ represents a linear or branched, optionally at least mono-substituted C₁₋₅-alkyl group, a saturated, optionally at least mono-substituted C₅₋₆-cycloaliphatic group, or an optionally at least mono-substituted phenyl group, more preferably R⁷ represents a linear or branched, optionally at least mono-substituted C₁₋₅-alkyl group, a saturated, optionally at least mono-substituted C₅₋₆-cycloaliphatic group,
or an optionally at least mono-substituted phenyl group, wherein in each case the substituents are independently from one another selected from the group consisting of a fluorine atom, a chlorine atom, a bromine atom, a methyl group, a methoxy group, a trifluoromethyl group and a trifluoromethoxy group.

16. The medicament according to claim 8, wherein R⁸ represents a linear or branched, saturated or unsaturated, optionally at least mono-substituted C₁₋₁₀-aliphatic group, a saturated or unsaturated, optionally at least mono-substituted, optionally at least one heteroatom as ring member containing C₃₋₈-cycloaliphatic group, which may be condensed with a mono- or polycyclic ring-system and/or which may be bonded via a linear or bridged C₁₋₁₀-alkylene group and/or which may be bridged by a linear or branched C₁₋₅-alkylene group, or an optionally at least mono-substituted 5- or 6-membered aryl or heteroaryl group, which may be condensed with a mono- or polycyclic ring system and/or which may be bonded via a linear or branched C₁₋₁₀-alkylene group, preferably R⁸ represents a linear or branched C₁₋₁₀-alkyl group, a saturated or unsaturated, optionally at least mono-substituted, optionally at least one heteroatom as ring member containing C₅₋₆-cycloaliphatic group, which may be condensed with a mono- or polycyclic ring-system and/or which may be bonded via a linear or bridged C₁₋₃-alkylene group and/or which may be bridged by a linear or branched C₁₋₃-alkylene group, or an optionally at least mono-substituted 5- or 6-membered aryl or heteroaryl group, which may be condensed with a mono- or polycyclic ring system and/or which may be bonded via a linear or branched C₁₋₃-alkylene group, more preferably R⁸ represents a methyl group, an ethyl group, an n-propyl group, an n-butyl group, an optionally at least mono-substituted phenyl group, an optionally at least mono-substituted benzyl group, an optionally at least mono-substituted naphthyl group, which may be bonded via a C₁₋₃-alkylene group, an optionally at least mono-substituted thienyl group, an optionally at least mono-substituted 2,1,3-Benzothiadiazole group, an optionally at least mono-substituted Benzo[b]thiophenyl group, an optionally at least mono-substituted Imidazo[2.1-b]thiazole group, an optionally at least mono-substituted 1H-pyrazole group or a 7,7-Dimethyl-2-oxo-bicyclo-[2.2.1]-hept-1-yl group, most preferably R⁸ represents a methyl group, an ethyl group, an n-propyl group, an n-butyl group, an optionally at least mono-substituted phenyl group, an optionally at least mono-substituted benzyl group, an optionally at least mono-substituted naphthyl group, which may be bonded via a C₁₋₃-alkylene group, an optionally at least mono-substituted thienyl group, an optionally at least mono-substituted 2,1,3-Benzothiadiazole group, an optionally at least mono-substituted Benzo[b]thiophenyl group, an optionally at least mono-substituted Imidazo[2.1-b]thiazole group, an optionally at least mono-substituted 1H-pyrazole group or a 7,7-Dimethyl-2-oxo-bicyclo-[2.2.1]-hept-1-yl group, wherein said substituents, if present, are identical or different and selected from the group consisting of a fluorine atom, a chlorine atom, a bromine atom, a methyl group, a formyl group, a phenyl group, a phenoxy group, a phenoxy group substituted with bromine in the 4-position and a methylsulfonyl group.

17. The medicament according to claim 8, wherein R⁹ represents an —SO₂—R¹²-moiety, a —CO—R³-moiety, a linear or branched, saturated or unsaturated, optionally at least mono-substituted C₁₋₁₀ aliphatic group, a saturated or unsaturated, optionally at least mono-substituted, optionally at least heteroatom as ring member containing C₃₋₈ cycloaliphatic group, which maybe condensed with a mono- or polycyclic ring system and/or which may be bonded by a linear or branched C₁₋₈ alkylene group and/or bridged by a linear or branched C₁₋₆ alkylene group, or an optionally at least mono-substituted aryl or heteroaryl group, which may be condensed with a mono- or polycyclic ring system and/or which may be bonded via a C₁₋₆ alkylene group, preferably R⁹ represents an —SO₂—R¹²-moiety, a linear or branched C₁₋₁₀ alkyl group, or an optionally at least mono-substituted phenyl group, which may be bonded via a C₁₋₂ alkylene group, more preferably R⁹ represents an —SO₂—R¹²-moiety, a linear or branched C₁₋₃ alkyl group, or a phenyl group, which may be bonded via a C₁₋₂ alkylene group and/or substituted with one or more substituents independently selected from the group consisting of a fluorine atom, a chlorine atom and a bromine atom.

18. The medicament according to claim 8, wherein R¹⁰ represents a linear or branched, saturated or unsaturated, optionally at least mono-substituted C₁₋₁₀-aliphatic group, a saturated or unsaturated, optionally at least mono-substituted, optionally at least one heteroatom as ring member containing C₃₋₈-cycloaliphatic group, which may be condensed with a mono- or polycyclic ring-system and/or which may be bonded via a linear or bridged C₁₋₁₀-alkylene group and/or which may be bridged by a linear or branched C₁₋₆-alkylene group, or an optionally at least mono-substituted 5- or 6-membered aryl or heteroaryl group, which may be condensed with a mono- or polycyclic ring system and/or which may be bonded via a linear or branched C₁₋₁₀-alkylene group, preferably R¹⁰ represents a linear or branched C₁₋₁₀-alkyl group, a saturated or unsaturated, optionally at least mono-substituted, optionally at least one heteroatom as ring member containing C₅₋₆-cycloaliphatic group, which may be condensed with a mono- or polycyclic ring-system and/or which may be bonded via a linear or bridged C₁₋₃-alkylene group and/or which may be bridged by a linear or branched C₁₋₃-alkylene group, or an optionally at least mono-substituted 5-or 6-membered aryl or heteroaryl group, which may be condensed with a mono- or polycyclic ring system and/or which may be bonded via a linear or branched C₁₋₃-alkylene group, more preferably R¹⁰ represents a methyl group, an ethyl group, an n-propyl group, an n-butyl group, an optionally at least mono-substituted phenyl group, an optionally at least mono-substituted benzyl group, an optionally at least mono-substituted naphthyl group, which may be bonded via a C₁₋₃-alkylene group, an optionally at least mono-substituted thienyl group, an optionally at least mono-substituted 2,1,3-Benzothiadiazole group, an optionally at least mono-substituted Benzo[b]thiophenyl group, an optionally at least mono-substituted Imidazo[2.1-b]thiazole group, an optionally at least mono-substituted 1H-pyrazole group or a 7,7-Dimethyl-2-oxo-bicylco-[2.2.1]-hept-1-yl group, most preferably R¹⁰ represents a methyl group, an ethyl group, an n-propyl group, an n-butyl group, an optionally at least mono-substituted phenyl group, an optionally at least mono-substituted benzyl group, an optionally at least mono-substituted naphthyl group, which may be bonded via a C₁₋₃-alkylene group, an optionally at least mono-substituted thienyl group, an optionally at least mono-substituted 2,1,3-Benzothiadiazole group, an optionally at least mono-substituted Benzo[b]thiophenyl group, an optionally at least mono-substituted Imidazo[2.1-b]thiazole group, an optionally at least mono-substituted 1H-pyrazole group or a 7,7-Dimethyl-2-oxo-bicyclo-[2.2.1]-hept-1-yl group, wherein said substituents, if present, are identical or different and selected from the group consisting of a fluorine atom, a chlorine atom, a bromine atom, a methyl group, a formyl group, a phenyl group, a phenoxy group, a phenoxy group substituted with bromine in the 4-position and a methylsulfonyl group.

19. The medicament according to claim 8, wherein R¹¹ represents a linear or branched, saturated or unsaturated, optionally at least mono-substituted C₁₋₁₀-aliphatic group, a saturated or unsaturated, optionally at least mono-substituted, optionally at least one heteroatom as ring member containing C₃₋₆-cyoloaliphatic group, which may be condensed with a mono- or polycyclic ring-system and/or which may be bonded via a linear or bridged C₁₋₁₀-alkylene group and/or which may be bridged by a linear or branched C₁₋₅-alkylene group, or an optionally at least mono-substituted 5- or 6-membered aryl or heteroaryl group, which may be condensed with a mono- or polycyclic ring system and/or which may be bonded via a linear or branched C₁₋₁₀-alkylene group, preferably R¹¹ represents a linear or branched C₁₋₁₀-alkyl group, a saturated or unsaturated, optionally at least mono-substituted, optionally at least one heteroatom as ring member containing C₅₋₆-cycloaliphatic group, which may be condensed with a mono- or polycyclic ring-system and/or which may be bonded via a linear or bridged C₁₋₃-alkylene group and/or which may be bridged by a linear or branched C₁₋₃-alkylene group, or an optionally at least mono-substituted 5- or 6-membered aryl or heteroaryl group, which may be condensed with a mono- or polycyclic ring system and/or which may be bonded via a linear or branched C₁₋₃-alkylene group, more preferably R¹¹ represents a methyl group, an ethyl group, an n-propyl group, an n-butyl group, an optionally at least mono-substituted phenyl group, an optionally at least mono-substituted benzyl group, an optionally at least mono-substituted naphthyl group, which may be bonded via a C₁₋₃-alkylene group, an optionally at least mono-substituted thienyl group, an optionally at least mono-substituted 2,1,3-Benzothiadiazole group, an optionally at least mono-substituted Benzo[b]thiophenyl group, an optionally at least mono-substituted imidazo[2.1-b]thiazole group, an optionally at least mono-substituted 1H-pyrazole group or a 7,7-Dimethyl-2-oxo-bicyclo-[2.2.1]-hept-1-yl group, most preferably R¹¹ represents a methyl group, an ethyl group, an n-propyl group, an n-butyl group, an optionally at least mono-substituted phenyl group, an optionally at least mono-substituted benzyl group, an optionally at least mono-substituted naphthyl group, which may be bonded via a C₁₋₃-alkylene group, an optionally at least mono-substituted thienyl group, an optionally at least mono-substituted 2,1,3-Benzothiadiazole group, an optionally at least mono-substituted Benzo[b]thiophenyl group, an optionally at least mono-substituted imidazo[2,1-b]thiazole group, an optionally at least mono-substituted 1H-pyrazole group or a 7,7-Dimethyl-2-oxo-bicyclo-[2.2.1]-hept-1 yl group, wherein said substituents, if present, are identical or different and selected from the group consisting of a fluorine atom, a chlorine atom, a bromine atom, a methyl group, a formyl group, a phenyl group, a phenoxy group, a phenoxy group substituted with bromine in the 4-position and a methylsulfonyl group.

20. The medicament according to claim 8, wherein R¹² represents a linear or branched, saturated or unsaturated, optionally at least mono-substituted C₁₋₁₀-aliphatic group, a saturated or unsaturated, optionally at least mono-substituted, optionally at least one heteroatom as ring member containing C₃₋₈-cycloaliphatic group, which may be condensed with a mono- or polycyclic ring-system and/or which may be bonded via a linear or bridged C₁₋₁₀-alkylene group and/or which may be bridged by a linear or branched C₁₋₆-alkylene group, or an optionally at least mono-substituted 5- or 6-membered aryl or heteroaryl group, which may be condensed with a mono- or polycyclic ring system and/or which may be bonded via a linear or branched C₁₋₁₀-alkylene group, preferably R¹² represents a linear or branched C₁₋₁₀-alkyl group, a saturated or unsaturated, optionally at least mono-substituted, optionally at least one heteroatom as ring member containing C₅₋₆-cycloaliphatic group, which may be condensed with a mono- or polycyclic ring-system and/or which may be bonded via a linear or bridged C₁₋₃-alkylene group and/or which may be bridged by a linear or branched C₁₋₃-alkylene group, or an optionally at least mono-substituted 5- or 6-membered aryl or heteroaryl group, which may be condensed with a mono- or polycyclic ring system and/or which may be bonded via a linear or branched C₁₋₃-alkylene group, more preferably R¹² represents a methyl group, an ethyl group, an n-propyl group, an n-butyl group, an optionally at least mono-substituted phenyl group, an optionally at least mono-substituted benzyl group, an optionally at least mono-substituted naphthyl group, which may be bonded via a C₁₋₃-alkylene group, an optionally at least mono-substituted thienyl group, an optionally at least mono-substituted 2,1,3-Benzothiadiazole group, an optionally at least mono-substituted Benzo[b]thiophenyl group, an optionally at least mono-substituted Imidazo[2.1-b]thiazole group, an optionally at least mono-substituted 1H-pyrazole group or a 7,7-Dimethyl-2-oxo-bicyclo-[2.2.1]-hept-1-yl group, most preferably R¹² represents a methyl group, an ethyl group, an n-propyl group, an n-butyl group, an optionally at least mono-substituted phenyl group, an optionally at least mono-substituted benzyl group, an optionally at least mono-substituted naphthyl group, which may be bonded via a C₁₋₃-alkylene group, an optionally at least mono-substituted thienyl group, an optionally at least mono-substituted 2,1,3-Benzothiadiazole group, an optionally at least mono-substituted Benzo[b]thiophenyl group, an optionally at least mono-substituted Imidazo[2.1-b]thiazole group, an optionally at least mono-substituted 1H-pyrazole group or a 7,7-Dimethyl-2-oxo-bicyclo-[2.2.1]-hept-1-yl group, wherein said substituents, if present, are identical or different and selected from the group consisting of a fluorine atom, a chlorine atom, a bromine atom, a methyl group, a formyl group, a phenyl group, a phenoxy group, a phenoxy group substituted with bromine in the 4-position and a methylsulfonyl group.

21. The medicament according to claim 8, wherein R¹³ represents a linear or branched, saturated or unsaturated, optionally at least mono-substituted C₁₋₁₀-aliphatic group, a saturated or unsaturated, optionally at least mono-substituted, optionally at least one heteroatom as ring member containing C₃₋₈-cycloaliphatic group, which may be condensed with a mono- or polycyclic ring-system and/or which may be bonded via a linear or bridged C₁₋₁₀-alkylene group and/or which may be bridged by a linear or branched C₁₋₅-alkylene group, or an optionally at least mono-substituted 5- or 6-membered aryl or heteroaryl group, which may be condensed with a mono- or polycyclic ling system and/or which may be bonded via a linear or branched C₁₋₁₀-alkylene group, preferably R¹³ represents a linear or branched C₁₋₁₀-alkyl group, a saturated or unsaturated, optionally at least mono-substituted, optionally at least one heteroatom as ring member containing C₅₋₆-cycloaliphatic group, which may be condensed with a mono- or polycyclic ring-system and/or which may be bonded via a linear or bridged C₁₋₃-alkylene group and/or which may be bridged by a linear or branched C₁₋₃-alkylene group, or an optionally at least mono-substituted 5- or 6-membered aryl or heteroaryl group, which may be condensed with a mono- or polycyclic ring system and/or which may be bonded via a linear or branched C₁₋₃-alkylene group, more preferably R¹³ represents a methyl group, an ethyl group, an n-propyl group, an n-butyl group, an optionally at least mono-substituted phenyl group, an optionally at least mono-substituted benzyl group, an optionally at least mono-substituted naphthyl group, which may be bonded via a C₁₋₃-alkylene group, an optionally at least mono-substituted thienyl group, an optionally at least mono-substituted 2,1,3-Benzothiadiazole group, an optionally at least mono-substituted Benzo[b]thiophenyl group, an optionally at least mono-substituted Imidazo[2.1-b]thiazole group, an optionally at least mono-substituted 1H-pyrazole group or a 7,7-Dimethyl-2-oxo-bicyclo-[2.2.1]-hept-1-yl group, most preferably R¹³ represents a methyl group, an ethyl group, an n-propyl group, an n-butyl group, an optionally at least mono-substituted phenyl group, an optionally at least mono-substituted benzyl group, an optionally at least mono-substituted naphthyl group, which may be bonded via a C₁₋₃-alkylene group, an optionally at least mono-substituted thienyl group, an optionally at least mono-substituted 2,1,3-Benzothiadiazole group, an optionally at least mono-substituted Benzo[b]thiophenyl group, an optionally at least mono-substituted Imidazo[2.1-b]thiazole group, an optionally at least mono-substituted 1H-pyrazole group or a 7,7-Dimethyl-2-oxo-bicyclo-[2.2.1]-hept-1-yl group, wherein said substituents, if present, are identical or different and selected from the group consisting of a fluorine atom, a chlorine atom, a bromine atom, a methyl group, a formyl group, a phenyl group, a phenoxy group, a phenoxy group substituted with bromine in the 4-position and a methylsulfonyl group.

22. The medicament according to claim 8 comprising at least one substituted azetidine compound of general formula I, 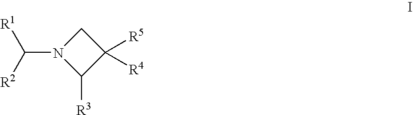
wherein
R¹ represents a phenyl group, said phenyl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, —OCF₃, —CN and —COOH,
R² represents a phenyl group, said phenyl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, —OCF₃, —CN and —COOH,
R³ represents a linear or branched, unsubstituted C₁₋₆ alkyl group,
R⁴ represents a hydrogen atom, —CN, —COOH, a linear or branched, unsubstituted C₁₋₆ alkyl group, or a phenyl group, said phenyl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, —OCF₃, —CN and —COOH,
R⁵ represents an —O—SO₂—R⁶-moiety, an —NH—CO—R⁷-moiety, an —NH₂-moiety, an —NH—SO₂—R⁸ moiety, an —NR⁹—SO₂—R¹⁰-moiety or an —O—C(═O)—R¹¹-moiety,
R⁶ represents a phenyl group, said phenyl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, —OCF₃, —CN and —COOH,
R⁷ represents a linear or branched C₁₋₆ alkyl group, said alkyl group being unsubstituted or substituted with 1, 2, 3, 4 or 5 substituents independently selected from the group consisting of F, Cl, Br, —OH, —SH, linear or branched C₁₋₆ alkoxy, —CF₃ and —OCF₃; a cyclopentyl or cyclohexyl group, said cyclopentyl or cyclohexyl group being unsubstituted or substituted with 1, 2, 3, 4 or 5 substituents independently selected from the group consisting of F, Cl, Br, —OH, —SH, linear or branched C₁₋₆ alkoxy, —CF₃ and —OCF₃; or a phenyl group, said phenyl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, —OCF₃, —CN and —COOH,
R⁸ represents a linear or branched C₁₋₅ alkyl group; an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, phenyl (optionally substituted with 1, 2, 3, 4 or 5 substituents independently selected from the group consisting of F, Cl, Br, OH, linear or branched C₁₋₆ alkyl and linear or branched C₁₋₆-alkoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alkoxy), —SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, ═O, OCF₃, —CN and —COOH;
R⁹ represents an —SO₂—R¹²-moiety; a —{C═O)—R¹³-moiety; a linear or branched C₁₋₆ alkyl radical; or a phenyl radical, said phenyl radical being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, —OCF₃, —CN and —COOH and/or said phenyl radical being optionally bonded via a C₁₋₃-alkylene bridge,
R¹⁰ represents a linear or branched C₁₋₅ alkyl group; an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, phenyl (optionally substituted with 1, 2, 3, 4 or 5 substituents independently selected from the group consisting of F, Cl, Br, OH, linear or branched C₁₋₆ alkyl and linear or branched (C₁₋₆-alkoxy), phenoxy (optionally substituted with 1, 2, 3, 4 or 5 substituents independently selected from the group consisting of F, Cl, Sr, OH, linear or branched C₁₋₆ alkyl and linear or branched C₁₋₆-alkoxy), —SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, ═O, —OCF₃, —CN and—COOH;
R¹¹ represents a linear or branched C₁₋₆ alkyl group; an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, phenyl (optionally substituted with 1, 2, 3, 4 or 5 substituents independently selected from the group consisting of F, Cl, Br, OH, linear or branched C₁₋₆ alkyl and linear or branched C₁₋₆-alkoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alkoxy), —SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, ═O, —OCF₃, —CN and —COOH;
R¹² represents a linear or branched C₁₋₆ alkyl group, an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆-alkoxy, phenyl (optionally substituted with 1, 2, 3, 4 or 5 substituents independently selected from the group consisting of F, Cl, Br, OH, linear or branched C₁₋₆ alkyl and linear or branched C₁₋₆-alkoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alkoxy), —SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, ═O, —OCF₃, —CN and —COOH; and
R¹³ represents a linear or branched C₁₋₆ alkyl group, an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, phenyl (optionally substituted with 1, 2, 3, 4 or 5 substituents independently selected from the group consisting of F, Cl, Br, OH, linear or branched C₁₋₆ alkyl and linear or branched C₁₋₆-alkoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alkoxy), —SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded, via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, ═O, —OCF₃, —CN and —COOH;
optionally in the form of one of the stereoisomers, preferably enantiomers or diastereomers, a racemate or in the form of a mixture of at least two of the stereoisomers, preferably enantiomers and/or diastereomers, in any mixing ratio, or a corresponding N-oxide thereof, a corresponding salt thereof, or a corresponding solvate thereof,
and optionally one or more pharmaceutically acceptable excipients.

23. The medicament according to claim 22 comprising at least one substituted azetidine compound of general formula I, 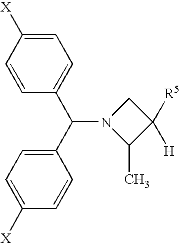
wherein
X represents a halogen atom, preferably a chlorine atom,
R represents an —O—SO₂—R⁶-moiety, an —NH—CO—R⁷-moiety, an —NH₂-moiety, an —NH—SO₂—R⁸ moiety, or an —NR⁹—SO₂—R¹⁰-moiety or an —O—C(═O)—R¹¹-moiety,
R⁶ represents a phenyl group, said phenyl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, —OCF₃, —CN and —COOH,
R⁷ represents a linear or branched C₁₋₆ alkyl group, said alkyl group being unsubstituted or substituted with 1, 2, 3, 4 or 5 substituents independently selected from the group consisting of F, Cl, Br, —OH, —SH, linear or branched C₁₋₆ alkoxy, —CF₃ and —OCF₃; a cyclopentyl or cyclohexyl group, said cyclopentyl or cyclohexyl group being unsubstituted or substituted with 1, 2, 3, 4 or 5 substituents independently selected from the group consisting of F, Cl, Br, —OH, —SH, linear or branched C₁₋₆ alkoxy, —CF₃ and —OCF₃; or a phenyl group, said phenyl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, —OCF₃, —CN and —COOH,
R⁸ represents a linear or branched C₁₋₅ alkyl group; an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, phenyl (optionally substituted with 1, 2, 3, 4 or 5 substituents independently selected from the group consisting of F, Cl, Br, OH, linear or branched C₁₋₆ alkyl and linear or branched C₁₋₆-alkoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alkoxy), —SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3,4 or 5 substituents independently selected from the group consisting of F, Cl, Br, linear or branched C₁₋₆ alkyl, linear or branched C₁₋₆ alkoxy, —(C═O)—H, —OH, —CF₃, ═O, —OCF₃, —CN and —COOH;
R⁹ represents an —SO₂—R¹² moiety; a —(C═O)—R¹³-moiety a linear or branched C₁₋₆ alkyl radical; or a phenyl radical, said phenyl radical being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, —OCF₃, —CN and —COOH and/or said phenyl radical being optionally bonded via a C₁₋₃-alkylene bridge,
R¹⁰ represents a linear or branched C₁₋₅ alkyl group; an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, phenyl (optionally substituted with 1, 2, 3, 4 or 5 substituents independently selected from the group consisting of F, Cl, Br, OH, linear or branched C₁₋₆ alkyl and linear or branched C₁₋₆-alkoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alkoxy), —SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, ═O, —OCF₃, —CN and —COOH;
R¹¹ represents a linear or branched C₁₋₅ alkyl group: an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆-alkoxy, phenyl (optionally substituted with 1, 2, 3, 4 or 5 substituents independently selected from the group consisting of F, Cl, Br, OH, linear or branched C₁₋₆ alkyl and linear or branched C₁₋₆-alkoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alkoxy), —SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, ═O, —OCF₃, —CN and —COOH;
R¹² represents a linear or branched C₁₋₅ alkyl group, an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, phenyl (optionally substituted with 1, 2, 3, 4 or 5 substituents independently selected from the group consisting of F, Cl, Br, OH, linear or branched C₁₋₆ alkyl and linear or branched C₁₋₆-alkoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alkoxy), —SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₆ alkyl, linear or branched C₁₋₆ alkoxy, —(C═O)—H, —OH, —CF₃, ═O, —OCF₃, —CN and —COOH; and
R¹³ represents a linear or branched C₁₋₅ alkyl group, an aryl or heteroaryl group selected from the group consisting of phenyl, naphthyl, thienyl, furanyl (furyl), 2,1,3-benzothiadiazole, benzo[b]thiophenyl, benzo[b]furanyl, imidazo[2.1-b]thiazole and pyrazole, said aryl or heteroaryl group being unsubstituted or substituted with 1, 2, 3, 4 or 5 substituents independently selected from the group consisting of F, Cl, Br, linear or branched C₁₋₆ alkyl, linear or branched C₁₋₆ alkoxy, phenyl (optionally substituted with 1, 2, 3, 4 or 5 substituents independently selected from the group consisting of F, Cl, Br, OH, linear or branched C₁₋₆-alkyl and linear or branched C₁₋₆-alkoxy), phenoxy (optionally substituted with 1, 2, 3, 4 or 5 substituents independently selected from the group consisting of F, Cl, Br, OH, linear or branched C₁₋₆ alkyl and linear or branched C₁₋₆-alkoxy), —SO₂—CH₃, —(C═O)—H, —OH, —CF₃, —OCF₃, —CN and —COOH and said aryl or heteroaryl group being optionally bonded via a C₁₋₃ alkylene group; or a cyclohexyl group, said cyclohexyl group being optionally bonded via a C₁₋₃ alkylene bridge and/or being optionally bridged by a linear or branched C₁₋₃ alkylene bridge and being unsubstituted or substituted with 1, 2, 3, 4 or 5 substituents independently selected from the group consisting of F, Cl, Br, linear or branched C₁₋₃ alkyl, linear or branched C₁₋₆ alkoxy, —(C═O)—H, —OH, —CF₃, ═O, —OCF₃, —CN and —COOH;
optionally in the form of one of the stereoisomers, preferably enantiomers or diastereomers, a racemate or in the form of a mixture of at least two of the stereoisomers, preferably enantiomers and/or diastereomers, in any mixing ratio, or a corresponding N-oxide thereof, a corresponding salt thereof, or a corresponding solvate thereof,
and optionally one or more pharmaceutically acceptable excipients.

24. The medicament according to claim 8 comprising at least one substituted azetidine compound of general formula I, 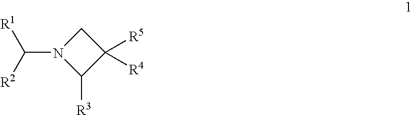
wherein
R¹ represents a phenyl group, which is mono-substituted with a halogen atom, preferably a chlorine atom, in the 4-position of the phenyl ring,
R² represents a phenyl group, which is mono-substituted with a halogen atom, preferably a chlorine atom, in the 4-position of the phenyl ring.
R³ represents a linear or branched, unsubstituted C₁₋₆ alkyl group, preferably a methyl group,
R⁴ represents a hydrogen atom,
R⁵ represents an —O—SO₂—R⁶-moiety, an —NH—CO—R⁷-moiety, an —NH₂-moiety, an —NH—SO₂—R⁸ moiety, or an —NR⁹—SO₂—R¹⁰-moiety,
R⁶ represents a phenyl ring, which is optionally substituted with one or more halogen atoms, preferably one or more fluorine and/or one or more chlorine atoms,
R⁷ represents a linear or branched C₁₋₅ alkyl group, a linear or branched C₁₋₅ alkyl group, which is at least partially fluorinated, a C₃₋₈ cycloalkyl group, or a phenyl group, which is optionally substituted with one or more halogen atoms, preferably one or more fluorine atoms,
R⁸ represents a linear or branched C₁₋₅ alkyl group,
a phenyl group, which is optionally substituted with one or more substituents independently selected from the group consisting of a fluorine atom, a chlorine atom, an unsubstituted phenyl group, a formyl group, a methylsulfonyl group, a benzyl group and a phenoxy group, which is optionally mono-substituted by a bromine atom in its 4-position,
a naphthyl group, which may be bonded via a methylene or ethylene group,
a Benzo[b]thiophene group, which is optionally substituted with one or more methyl groups and/or one or more chlorine atoms,
a pyrazole group, which is optionally substituted with one or more substituents independently selected from the group consisting of a methyl group, an ethyl group and a phenyl group,
an imidazo[2,1-b]thiazole group, which is optionally substituted with one or more chlorine atoms,
a thienyl group, a furyl group, a 2,1,3-Benzothiadiazole group, a 7,7-Dimethyl-2-oxo-bicyclo-[2.2.1]-hept-1-yl group, or a benzyl group,
R⁹ represents a C₁₋₆ alkyl group, preferably a methyl group, a phenyl group, which is optionally substituted with one or more fluorine atoms and/or one or more chlorine atoms, a benzyl group, wherein the ring is optionally substituted with one or more fluorine atoms and/or one or more chlorine atoms, or a —SO₂—R¹²-moiety,
R¹⁰ represents a phenyl group, which is optionally substituted with one or more fluorine atoms and/or one or more chlorine atoms,
R¹²represents a C₁₋₅ alkyl group, preferably a methyl group, or a phenyl group, which is optionally substituted with one or more fluorine atoms and/or one or more chlorine atoms, and
optionally in the form of one of the stereoisomers, preferably enantiomers or diastereomers, a racemate or in the form of a mixture of at least two of the stereoisomers, preferably enantiomers and/or diastereomers, in any mixing ratio, or a corresponding N-oxide thereof, a corresponding salt thereof, or a corresponding solvate thereof,
and optionally one or more pharmaceutically acceptable excipients.

25. The medicament according to claim 8, wherein the substituted azetidine compound is selected from the group consisting of
4-Fluoro-benzenesuffonic acid 1-[trans-bis-(4-chloro-phenyl)-methyl]-2-methyl-azetidin-3-yl ester,
N-{(2S ,3R)-1-[Bis-(4-chloro-phenyl)-methyl]-2-methyl-azetidin-3-yl}-2,2,2-trifluoro-acetamide,
(2S,3R)-1-[Bis-(4-chloro-phenyl)-methyl]-2-methyl-azetidin-3-ylamine,
Hexanoic acid {1-[bis-(4-chloro-phenyl)-methyl]-2-methyl-azetidin-3-yl}-amide,
N-{(2S,3R)-1-[Bis-(4-chloro-phenyl)-methyl]-2-methyl-azetidin-3-yl}-4-fluoro-benzenesulfonamide,
Thiophene-2-sulfonic acid {(2S,3R)-1-[bis-(4-chloro-phenyl)-methyl]-2-methyl-azetidin-3-yl}-amide,
Cyclohexanecarboxylic acid {(2S,3R)-1-[bis(4-chloro-phenyl)-methyl]-2-methyl-azetidin-3-yl}-amide,
Butane-1-sulfonic acid {(2S,3R)-1-[bis-(4-chloro-phenyl)-methyl]-2-methyl-azetidin-3-yl}-amide,
N-{(2S,3R)-1-[Bis-(4-chloro-phenyl)-methyl)-2-methyl-azetidin-3-yl}-3,5-difluoro-benzamide,
Naphthalene-2-sulfonic acid {trans-1-[bis-(4-chloro-phenyl)-methyl]-2-methyl-azetidin-3-yl}-amide,
Biphenyl-4-sulfonic acid {trans-1-[bis-(4-chloro-phenyl)-methyl]-2-methyl-azetidin-3-yl}-amide,
4-Acetyl-N-{trans-1-[bis-(4-chloro-phenyl)-methyl]-2-methyl-azetidin-3-yl}-benzenesulfonamide,
N-(trans-1-[Bis-{4-chloro-phenyl)-methyl]-2-methyl-azetidin-3-yl}-4-(4-bromo-phenoxy)-benzenesulfonamide,
N-(trans-1-[Bis-(4-chloro-phenyl)-methyl]-2-methyl-azetidin-3-yl}-4-methylsulfonyl-benzenosulfonamide,
2,1,3-Benzothiadiazole-4-sulfonic acid {trans-1-[bis-(4-chloro-phenyl)-methyl]-2-methyl-azetidin-3-yl}-amide,
5-Chloro-3-methyl-benzo[b]thiophene-2-sulfonic acid {trans-1-[bis-(4-chloro-phenyl)-methyl]-2-methyl-azetidin-3-yl}-amide,
6-Chloro-Imidazo[2,1-b]thiazole-5-sulfonic acid {trans-1-[bis-(4-chlorophenyl)-methyl]-2-methyl-azetidin-3-yl}-amide,
N-{trans-1-[Bis-(4-chloro-phenyl)-methyl]-2-methyl-azetidin-3-yl}-3,5-dichloro-benzenesulfonamide,
2-Naphthalene-1-yl-ethanesulfonic acid {trans-1-[bis-(4-chloro-phenyl)-methyl]-2-methyl-azetidin-3-yl}-amide,
N-{trans-1-[Bis-(4-chloro-phenyl)-methyl]-2-methyl-azetidin-3-yl)-phenyl-methylsulfonamide,
N-{trans-1-[Bis-(4-chloro-phenyl)-methyl]-2-methyl-azetidin-3-yl}-(7,7-dimethyl-2-oxo-bicyclo[2.2.1]hept-1-yl)-methylsulfonamide,
Naphthalene-1-sulfonic acid {trans-1-[bis-(4-chloro-phenyl)-methyl]-2-methyl-azetidin-3-yl}-amide,
N-{trans-1-(Bis-(4-chloro-phenyl)-methyl]-2-methyl-azetidin-3-yl}-4-phenoxy-benzenesulfonamide),
1,3,5-Trimethyl-1H-pyrazole-4-sulfonic acid {trans-1-[bis-(4-chloro-phenyl)-methyl]-2-methyl-azetidin-3-yl}-amide,
Benzo[b]thiophene-3-sulfonic acid {trans-1-[bis-(4-chloro-phenyl)-methyl]-2-methyl-azetidin-3-yl}-amide,
5-Methyl-1-phenyl-1H-pyrazole-4-sulfonic acid {trans-1-[bis-(4-chlorophenyl)-methyl]-2-methyl-azetidin-3-yl}-amide,
N-{trans-1-[Bis-(4-chloro-phenyl)-methyl]-2-methyl-azetidin-3-yl)-N-methyl-4-fluoro-benzenesulfonamide,
N-{trans-1-[Bis-(4-chloro-phenyl)-methyl]-2-methyl-azetidin-3-yl}-N-(4-fluoro-benzyl)-4-fluoro-benzenesulfonamide,
N-{trans-1-[Bis-(4-chloro-phenyl)-methyl]-2-methyl-azetidin-3-yl}-N-propyl-4-fluoro-benzenesulfonamide,
N-{trans-1-[Bis-(4-chloro-phenyl)-methyl]-2-methyl-azetidin-3-yl}-N-(methylsulphonyl)-4-fluoro-benzenesulfonamide,
N-{trans-1-[Bis-(4-chloro-phenyl)-methyl]-2-methyl-azetidin-3-yl}-N-bis(4-fluoro-benzenesulfonamide),
N-[(trans-1-[Bis-(4-chloro-phenyl)-methyl]-2-methyl-azetidin-3-yl}-4-fluoro-benzenesulfonamide, and
N-[(2R,3S)-1-[Bis-(4-chloro-phenyl)-methyl]-2-methyl-azetidin-3-yl}-4-fluoro-benzenesulfonamide;
optionally in the form of a corresponding N-oxide, a corresponding salt or a corresponding solvate.

26. The medicament according to claim 8, wherein the substituted azetidine compound is present in an amount effective for the modulation of cannabinoid-receptors, preferably cannabinoid 1 (CB₁) receptors, for the prophylaxis and/or treatment of disorders of the central nervous system, disorders of the immune system, disorders of the cardiovascular system, disorders of the endocrinous system, disorders of the respiratory system, disorders of the gastrointestinal tract or reproductive disorders.

27. The medicament according to claim 8, wherein the substituted azetidine compound is present in an amount effective for the prophylaxis and/or treatment of food intake disorders, preferably selected from the group consisting of bulimia, anorexia, cachexia, obesity and type II diabetus mellitus (non-insulin dependent diabetes mellitus), more preferably obesity.

28. The medicament according to claim 8, wherein the substituted azetidine compound is present in an amount effective for the prophylaxis and/or treatment of psychosis.

29. The medicament according to claim 8, wherein the substituted azetidine compound is present in an amount effective for the prophylaxis and/or treatment of alcohol abuse and/or alcohol addiction, nicotine abuse and/or nicotine addiction, drug abuse and/or drug addiction and/or medicament abuse and/or medicament addiction, preferably drug abuse and/or drug addiction.

30. The medicament according to claim 8, wherein the substituted azetidine compound is present in an amount effective for the prophylaxis and/or treatment of one or more disorders selected from the group consisting of schizophrenia, anxiety, depression, epilepsy, neurodegenerative disorders, cerebral disorders, spinocerebellar disorders, cognitive disorders, cranial trauma, panic attacks, peripheric neuropathy, glaucoma, migraine, Morbus Parkinson, Morbus Huntington, Morbus Alzheimer, Raynaud's disease, tremblement disorders, compulsive disorders, senile dementia, thymic disorders, tardive dyskinesia, bipolar disorders, cancer, medicament-induced movement disorders, dystonia, endotoxemic shock, hemorragic shock, hypotension, insomnia, immunologic disorders, sclerotic plaques, vomiting, diarrhea, asthma, memory disorders, pruritus, pain or for potentiation of the analgesic effect of narcotic and non-narcotic analgesics, or for influencing intestinal transit.

31. A medicament comprising at least one substituted azetidine compound as defined in claim 8 and optionally one or more pharmaceutically acceptable excipients, wherein the substituted azetidine compound is present in an amount effective for the modulation of cannabinoid-receptors, preferably cannabinold 1 (CB₁) receptors, for the prophylaxis and/or treatment of disorders of the central nervous system, disorders of the immune system, disorders of the cardiovascular system, disorders of the endocrinous system, disorders of the respiratory system, and disorders of the gastrointestinal tract or reproductive disorders.

32. A medicament comprising at least one substituted azetidine compound as defined in claim 8 and optionally one or more pharmaceutically acceptable excipients, wherein the substituted azetidine compound is present in an amount effective for the prophylaxis and/or treatment of food intake disorders, preferably selected from the group consisting of bulimia, anorexia, cachexia, obesity and type II diabetus mellitus (non-insulin dependent diabetes mellitus), more preferably obesity.

33. A medicament comprising at least one substituted azetidine compound as defined in claim 8 and optionally one or more pharmaceutically acceptable excipients, wherein the substituted azetidine compound is present in an amount effective for the prophylaxis and/or treatment of psychosis.

34. A medicament comprising at least one substituted azetidine compound as defined in claim 8 and optionally one or more pharmaceutically acceptable excipients, wherein the substituted azetidine compound is present in an amount effective for the prophylaxis and/or treatment of alcohol abuse and/or alcohol addiction, nicotine abuse and/or nicotine addiction, drug abuse and/or drug addiction and/or medicament abuse and/or medicament addiction, preferably drag abuse and/or drug addiction.

35. A medicament comprising at least one substituted azetidine compound according to claim 8 and optionally one or more pharmaceutically acceptable excipients, wherein the substituted azetidine compound is present in an amount effective for the prophylaxis and/or treatment of one or more disorders selected from the group consisting of schizophrenia, anxiety, depression, epilepsy, neurodegenerative disorders, cerebral disorders, spinocerebellar disorders, cognitive disorders, cranial trauma, panic attacks, peripheric neuropathy, glaucoma, migraine, Morbus Parkinson, Morbus Huntington, Morbus Alzhelmer, Raynaud's disease, tremblement disorders, compulsive disorders, senile dementia, thymic disorders, tardive dyskinesia, bipolar disorders, cancer, medicament-induced movement disorders, dystonia, endotoxemic shock, hemorragic shock, hypotension, insomnia, immunologic disorders, sclerotic plaques, vomiting, diarrhea, asthma, memory disorders, pruritus, pain, or for potentiation of the analgesic effect of narcotic and non-narcotic analgesics, or for influencing intestinal transit.
